(19) United States
(12) Patent Application Publication
(10) Pub. No.: US 2014/0081146 A1
(43) Pub. Date: Mar. 20, 2014

(54) BONE MINERAL DENSITY MEASUREMENT APPARATUS AND METHOD

(71) Applicants: Alan R. Keim, New Hope, PA (US); David Comley, New Hope, PA (US); Jonathan Singer, New Hope, PA (US)

(72) Inventors: Alan R. Keim, New Hope, PA (US); David Comley, New Hope, PA (US); Jonathan Singer, New Hope, PA (US)

(21) Appl. No.: 14/022,941

(22) Filed: Sep. 10, 2013

Related U.S. Application Data
(60) Provisional application No. 61/700,736, filed on Sep. 13, 2012.

(51) Int. Cl. A61B 5/00

(52) U.S. Cl. CPC A61B 5/4509; A61B 5/0082; A61B 5/70
USPC 600/476; 600/300; 600/407

(57) ABSTRACT

A support mechanism may maintain a middle phalanx in a fixed position relative to an imaging sensor/receptor during a bone mineral density (BMD) test. The mechanism may comprise a flat hand plate and a cover. The cover may be shaped so that it guides the finger towards the target area of the receptor. The cover may be raised slightly above the hand plate. A hand may be placed in the mechanism with the palm facing downwards, resting on the hand plate, and the middle finger raised and resting flat on an imaging receptor. A musculoskeletal response may ensure that the middle phalanx remains proximate the imaging receptor for the duration of the BMD Test.

### CROSS REFERENCE TO RELATED APPLICATIONS

The instant application claims priority to U.S. provisional patent application No. 61/700,736, filed Sep. 13, 2012. U.S. provisional patent application No. 61/700,736 is incorporated herein by reference in its entirety.

### TECHNICAL FIELD

The technical field generally relates to measuring bone mineral density and bone mineral content.

### BACKGROUND

Poor bone density is known to be a contributing factor of fractures. Fractures resulting from poor bone density are not uncommon in elderly persons and post-menopausal women. Because many fractures are a result of falls, fractures of the leg and pelvis are common. These types of fractures can lead to increased medical costs, an inability to live independently, and even risk of death.

Bone density tests can be cumbersome, can require large equipment, and can expose individuals to large amounts of radiation.

### SUMMARY

The following presents a simplified summary that describes some aspects or embodiments of the subject disclosure. This summary is not an extensive overview of the disclosure. Indeed, additional or alternative embodiments of the subject disclosure may be available beyond those described in the summary.

A device for measuring bone mineral density (BMD), referred to herein as a densitometer, and also referred to as the AccuDEXA® densitometer or Accudxa2® densitometer, in an example embodiment, comprises a peripheral Dual-Energy X-ray (p-DXA) screening device. The densitometer may provide an estimate of BMD. The densitometer may provide an estimate of Bone Mineral Content (mass). The densitometer may facilitate a determination of standardized t-scores. The densitometer may facilitate a determination of standardized z-scores.

A t-score may represent a measure of a patient's BMD compared with a young healthy normal population (ages 20-29) of the same gender and ethnicity. The t-score may be indicated in terms of the number of standard deviations above (positive t-score) or below (negative t-score) the mean reference BMD.

A z-score may represent a measure of how the BMD of an individual patient compares with the BMD of a reference population of the same age group, gender, and ethnicity. The z-score may indicated as the number of standard deviations above (positive z-score) or below (negative z-score) the mean BMD of an age-matched control.

In an example embodiment, a screening Region of Interest (ROI) may be a middle phalanx of a middle finger of a non-dominant hand. BMD measurements obtained via the densitometer on the middle phalanx of the middle finger may be used to estimate BMD for other sites, such as for example, the hip. As the finger is easily accessible as a measurement site, a test may take very little time to complete (e.g., less than 1 minute) and the patient may be exposed to a low absorbed dose of x-ray radiation (e.g., approximately 3.76×10⁻⁴ microSieverts per exam). No protective garments are required, either for the patient or the operator, because the x-ray radiation levels are extremely low. The densitometer may provide results within 60 seconds and allow an operator to distinguish between osteoporotic, pre-osteoporotic and normal bone density states.

In an example embodiment, to position a finger for measurement, a patient's hand may be inserted into the device and a laser line may be projected onto the skin. The hand may be moved into the unit until the projected laser line bisects the joint between the intermediate and distal phalanxes. The wrinkled skin above this joint may be used to determine that the finger is properly positioned.

The densitometer may be used as a screening tool. For example, the densitometry may be used as a screening tool for bone density disorders in women and men of any age for which an appropriate normative database may exist in the densitometer.

### BRIEF DESCRIPTION OF THE DRAWINGS

The following detailed description of preferred embodiments is better understood when read in conjunction with the appended drawings. For the purposes of illustration, there is shown in the drawings exemplary embodiments; however, the subject matter is not limited to the specific elements and instrumentalities disclosed.

FIG. 1 depicts an example embodiment of the herein described densitometer.

FIG. 2 is an example photographic depiction of an example positioning mechanism.

FIG. 3 is an example illustrative depiction of an example positioning mechanism.

FIG. 4 depicts example positioning of a hand.

FIG. 5 illustrates an example densitometer overall system logical architecture.

FIG. 6 illustrates an example block diagram of the densitometer hardware environment.

FIG. 7 illustrates an example module table for the densitometer.

FIG. 8 illustrates an example task model for the densitometer.

FIG. 9 illustrates an example endpoint definition table for the densitometer.

FIG. 10 illustrates example control byte descriptions for the densitometer.

FIG. 11 illustrates example status byte descriptions for the densitometer.

FIG. 12 illustrates an example state transition diagram for the densitometer.

FIG. 13 illustrates example input/output (I/O) descriptions for an example power/audio controller of the densitometer.

FIG. 14 depicts example pseudo code.

FIG. 15 illustrates example input/output (I/O) descriptions for an example filter arm module of the densitometer.

FIG. 16 depicts example pseudo code.

FIG. 17 illustrates an example task model for the densitometer.

FIG. 18 depicts an example graphical user interface (GUI) menu structure for the densitometer.

FIG. 19, FIG. 20, FIG. 21, FIG. 22, FIG. 23, FIG. 24, FIG. 25, FIG. 26, FIG. 27, FIG. 28, FIG. 29, FIG. 30, and FIG. 31 depict an example application flow diagram for user functions of the densitometer.

FIG. 32 and FIG. 33 depict example BMD test reports.

FIG. 34 depicts an example upgrade menu.

FIG. 35 depicts an example normative database.

FIG. 36 is an example depiction of a front view of an example embodiment of the densitometer.

FIG. 37 is a depiction of a back view of an example embodiment of the densitometer.

FIG. 38 is a block diagram of an example configuration of the densitometer coupled to a printer.

FIG. 39 is an example illustration of some of the on-screen features based on age of the AccuDEXA® densitometer appear below using the Age.

FIG. 40 depicts example positioning of a hand.

FIG. 41, FIG. 42, FIG. 43, FIG. 44, FIG. 45, FIG. 46, FIG. 47, FIG. 48, FIG. 49, and FIG. 50 depict an example process for using the AccuDEXA® densitometer.

FIG. 51, FIG. 52, and FIG. 53 show examples of bone densitometry reports.

FIG. 54 depicts example t-score and z-score calculations.

FIG. 55 depicts sample graphs of t-scores versus age.

FIG. 56, FIG. 57, and FIG. 58, illustrate example densitometry reports.

FIG. 59, FIG. 60, FIG. 61, FIG. 62, FIG. 63, FIG. 64, and FIG. 65 depict an example process for using the AccuDEXA® densitometer.

FIG. 66, FIG. 67, FIG. 68, FIG. 69, and FIG. 70 illustrate an example process for performing a phantom test.

FIG. 71 and FIG. 72 depict example phantom test reports.

FIG. 73 and FIG. 74 depict and example process for performing a system test.

FIG. 75 depicts an example duty cycle.

FIG. 76 and FIG. 77 depicts example specifications.

FIG. 78, FIG. 79, FIG. 80, FIG. 81, and FIG. 82 illustrate an example process for printing a patient log report.

FIG. 83, FIG. 84, FIG. 85, FIG. 86, FIG. 87, and FIG. 88 illustrate an example process for copying a patient log report.

FIG. 89, FIG. 90, and FIG. 91 depict example error messages.

FIG. 92 is an example depiction of a front view of an example embodiment of the densitometer.

FIG. 93 is a depiction of a back view of an example embodiment of the densitometer.

FIG. 94 is a block diagram of an example configuration of the densitometer coupled to a printer and/or USB Thumb Drive.

FIG. 95 is an example illustration of example on-screen.

FIG. 96 depicts example correct finger positioning for a BMD Test.

FIG. 97 is a flow chart of an example process for positioning and measuring BMD.

FIG. 98, FIG. 99, FIG. 100, FIG. 101, FIG. 102, FIG. 103, FIG. 104, FIG. 105, FIG. 106, FIG. 107, FIG. 108, and FIG. 109 depict an example process for using the Accudxa2® densitometer.

FIG. 110, FIG. 111, and FIG. 112 show examples of bone densitometry reports.

FIG. 113 depicts sample graphs of t-scores versus age.

FIGS. 114 and 115 illustrate example densitometry reports.

FIGS. 116 and 117 depict an example process for reviewing stored BMD Test Reports on the glass-on-glass color LCD and/or an externally connected printer.

FIGS. 118 and 119 depict an example process for setting the date and the time stored in the processor of the Accudxa2®.

FIG. 120 illustrates an example process for using the Accudxa2® to print a test report on an externally connected printer.

FIG. 121 illustrates an example of a test report printed on the Accudxa2® using an externally connected printer.

FIG. 122, FIG. 123, FIG. 124, FIG. 125, and FIG. 126 illustrate an example process for performing a phantom test.

FIGS. 127 and 128 depict example phantom test reports.

FIG. 129, FIG. 130, FIG. 131, and FIG. 132 depict an example process for performing a system test.

FIG. 133 depicts an example process for performing a software upgrade of the Accudxa2®.

FIG. 134 and FIG. 135 depict and electrical summary.

FIG. 136, FIG. 137, and FIG. 138 illustrate an example process for printing a patient log report.

FIG. 139, FIG. 140, and FIG. 141 illustrate an example process for copying a patient log report.

FIG. 142, FIG. 143, and FIG. 144 depict example error messages.

### DETAILED DESCRIPTION OF ILLUSTRATIVE EMBODIMENTS

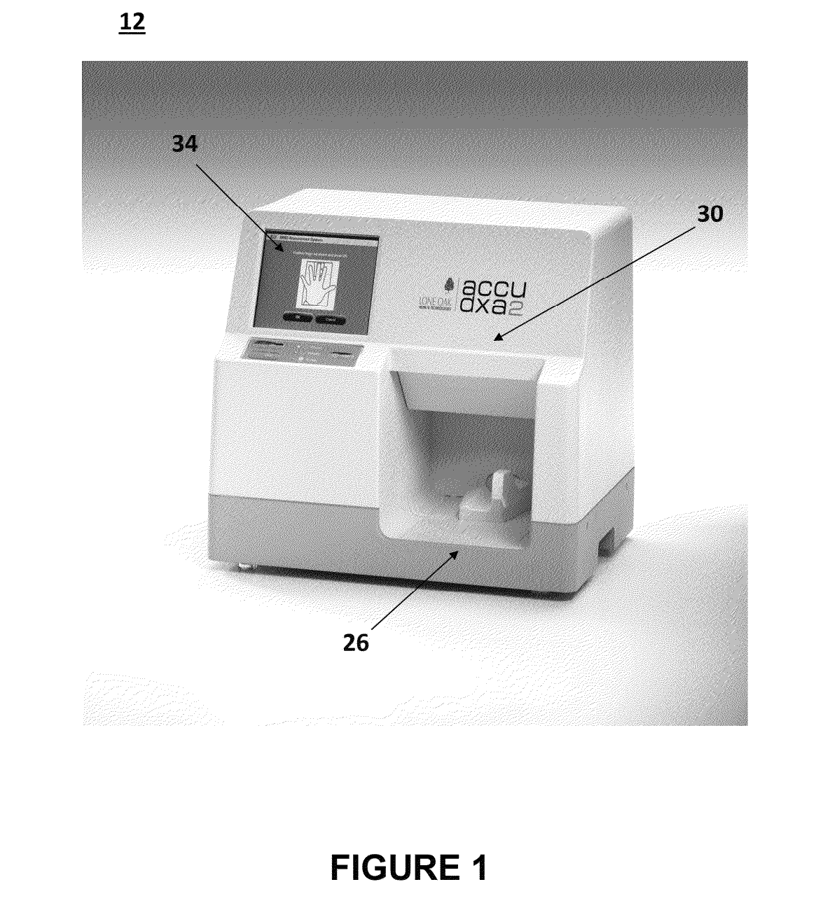

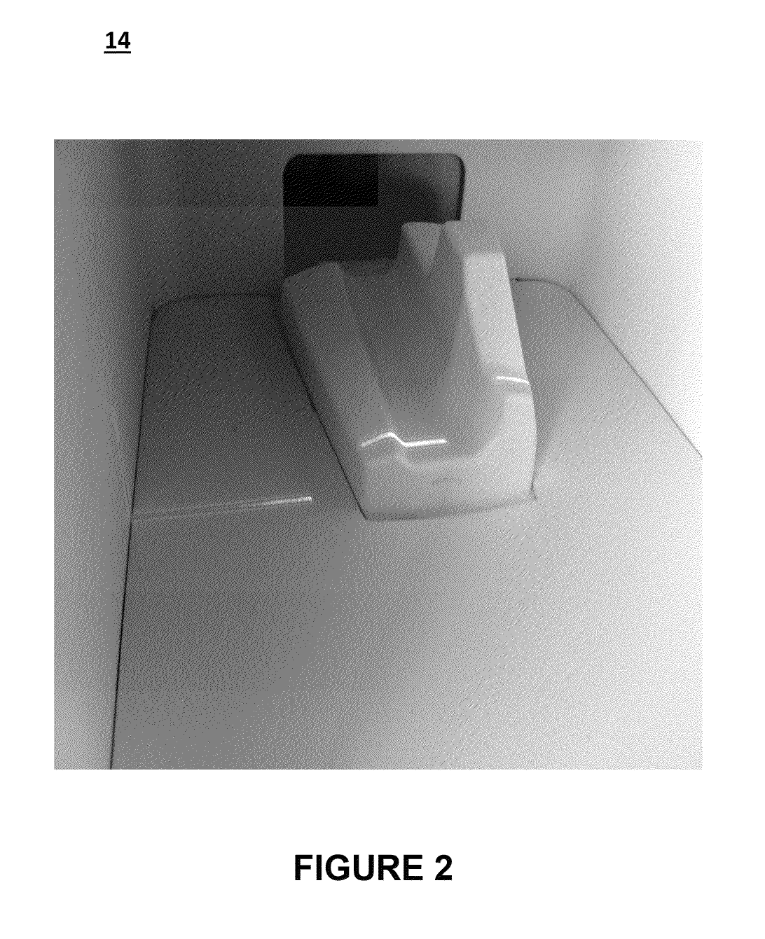

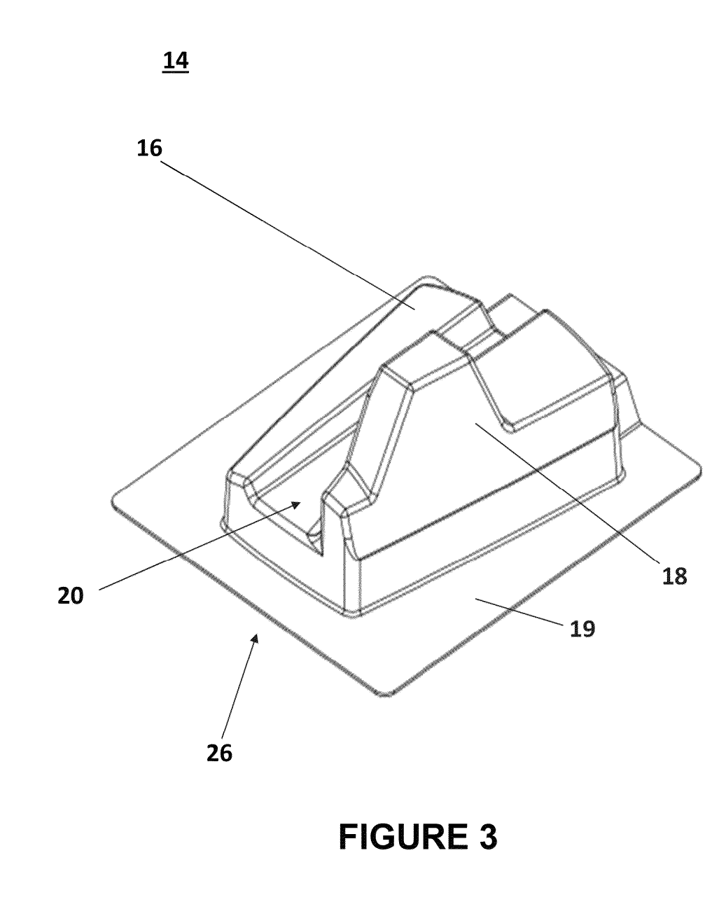

FIG. 1 is a pictorial depiction of an example embodiment of the herein described densitometer 12. In an example embodiment, the densitometer 12 may comprise a positioning mechanism 14 as depicted in FIG. 2 and FIG. 3. FIG. 2 is a pictorial depiction of an example embodiment of the positioning mechanism 14. FIG. 3 is an isometric view illustration of an example embodiment of the positioning mechanism 14.

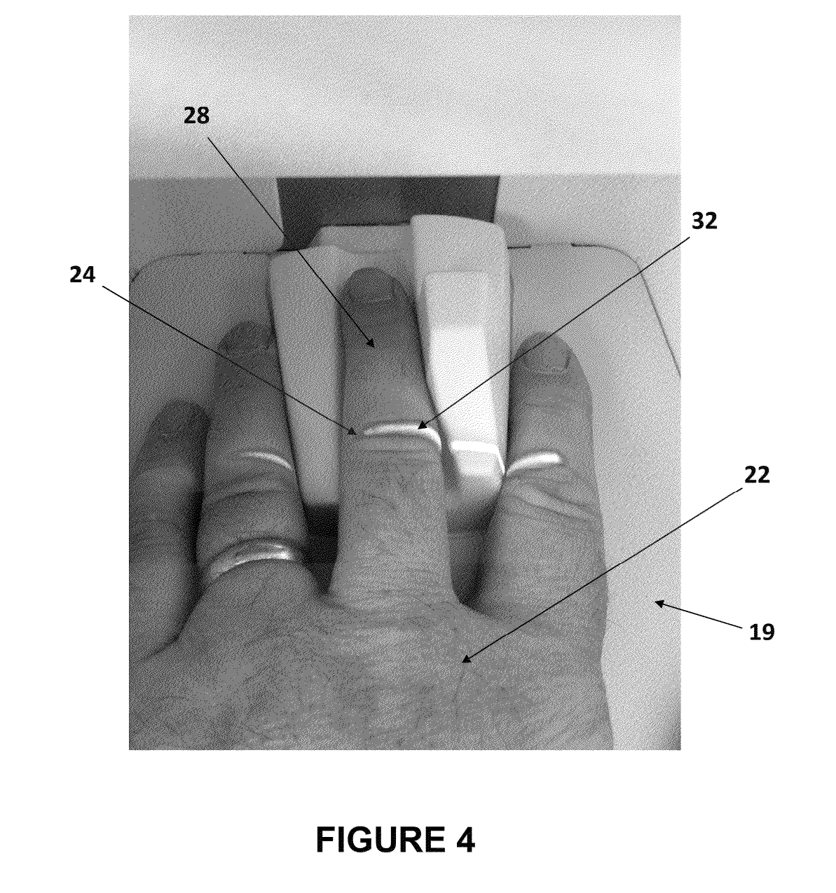

As shown in FIG. 3, the positioning mechanism 14 may comprise protruding portions 16 and 18 with recessed well 20 positioned therebetween. The position mechanism may comprise a hand plate 19 on which the palm of a hand may rest. The hand plate 19 may be a supporting plate that supports the palm of the hand in order to align a finger in the recessed portion 20. In an example embodiment, a finger may be placed within the recessed portion 20. A surface of the recessed well portion 20 may be concave in order to conform to the shape of a finger. FIG. 4 is an example depiction of hand positioning in the densitometer 12. As described in more detail herein, the densitometer 12 may comprise an x-ray generator and an imaging sensor (e.g., CMOS imaging sensor) to accurately image a Region of Interest (ROI) 20 at two different energy levels. A hand (e.g., a patient's hand) 22 may be positioned in the device 12 (e.g., within well 20) such that the ROI 24 is centered over an imaging sensor (e.g., CMOS imaging sensor), as depicted in FIG. 4. The imaging sensor may be placed proximate to and under the well 20, as depicted by arrow 26 in FIG. 3. A finger position protocol may be utilized to ensure that a finger 28 is placed correctly over the CMOS imaging sensor. Once the finger 28 is properly positioned, an energy source (e.g., x-ray source) may be activated at two different energy levels and two separate images acquired from the imaging sensor by an embedded processor, or processors, in the device. In an example embodiment, when a finger is properly positioned, the energy source may be positioned proximate to and above the ROI 24. FIG. 1 depicts example regions at which the energy source and the imaging sensor may be positioned in the densitometer 12. Region 26 depicts an example region at which the imaging sensor may be positioned. Region 30 depicts an example region at which the energy source may be positioned.

In an example configuration, as depicted in FIG. 1, the densitometer may comprise a color glass-on-glass touchscreen 34 for entering patient information. A precise and repeatable finger positioning protocol, controlled by the densitometer 12, may use a laser guide to ensure that the finger 28 is positioned correctly. Once the finger 28 is properly positioned, the x-ray source may be activated at two different energy levels and two separate x-ray images acquired and analyzed. The bone mineral density reading may be displayed (expressed in g/cm2).

Upon obtaining images, the soft tissue component from each pixel in the ROI images may be eliminated via instructions executed by a processor, or processors, of the densitometer 12. The mass of the bone in each pixel also may be determined mathematically by instructions executed by a processor, or processors, of the densitometer 12. The outline of the bone may be determined mathematically. The bone mass may be divided by the bone area to provide a real-time estimate of bone density expressed in g/cm².

In an example embodiment, the positioning mechanism 14 may be used to position an intermediate phalanx thereon. When the patient's finger 28 is positioned over the receptor as depicted in FIG. 4, controlling software may activate the laser line generator positioned above the finger and may provide a prompt to insert the patient's hand into the densitometer 12. The laser line generator may be aligned with one end of the imaging sensor. The imaging sensor may comprise any appropriate type of imaging sensor, such as, for example, a CMOS a CCD imaging sensor, or the like. The patient's hand may be inserted into the device and a laser line 32 may be projected onto the skin. The hand 22 may be moved into the unit until the projected laser line 32 bisects the joint between the intermediate and proximal phalanxes. The skin above this joint wrinkles naturally and may be observed to determine where the middle of the joint is by inspecting the wrinkles in the skin.

Once the finger 28 has been appropriately positioned in the positioning mechanism 14, an energy beam (e.g., x-ray beam) may be radiated. The energy beam may be initiated and/or controlled by instructions executed by a processor, or processors, of the densitometer 12, and activated by, for example, a Scan pushbutton switch, or the like. The energy beam may expose imaging sensor receptor(s) and captures a low-energy image which may be displayed to the operator via any appropriate mechanism, such as, for example, an LCD touchscreen (See example touchscreen 26 in FIG. 1). Correct positioning of the phalanx over the imaging sensor may be confirmed. If the position is correct, the operator may accept the finger positioning image and the BMD test may commence. If the finger 28 is mis-positioned, the finger may be repositioned, and the positioning process may be repeated. In an example embodiment, an operator, or the like, may instruct the patient to reposition the patient's finger 28 to correct placement, and the operator may repeat the positioning process.

It is to be understood that although the imaging sensor is described herein as a CMOS imaging sensor, the imaging sensor is not limited thereto. The imaging sensor may comprise any appropriate technology, circuitry, hardware, software, etc. in order to perform imaging sensor functions as described herein. It is to be understood that although an energy source is described herein as an x-ray energy source, the energy source is not limited thereto. The energy source may comprise any appropriate technology, circuitry, hardware, software, etc. in order to perform energy source functions as described herein. It is to be understood that, as described herein, the number of images obtained is two, the number of obtained images is not limited thereto. The number of obtained image may comprise any appropriate number (e.g., one, greater than one). It is to be understood that, as described herein, the number of energy levels utilized is two, the number of energy levels utilized is not limited thereto. The number of energy levels may comprise any appropriate number (e.g., one, greater than one).

The densitometer may contain a normative database that may be used to calculate a t-score and/or a z-score for the patient. These scores may compare the patient's BMD reading to the mean BMD value for a population of patients of the same gender and ethnicity. The scores may be expressed in standard deviations from the mean BMD value. The t-score may be compared against three cutoff values to determine whether the patient may have osteoporosis (−2.5 SD below the mean value for the Young Healthy Normal (YHN) population), low bone mass (−1 to −2.5 SD below the YHN mean value) or a normal reading (−1 or more SD below the YHN mean value). The z-score may be calculated in a similar fashion but the z-score compares the patient's BMD to average values for a population of the same age as the patient.

The following sections describe an example overall densitometer design architecture; module dependency; operation flow; thread design, and endport usage. As described herein:
BMD refers to Bone Mineral Density, which may be interpreted as an area-based estimate of the density of bone.
HVPS refers to High Voltage Power Supply. A power converter generating about 200 VAC at 20 KHz to energize the x-ray tube head.
Sensor refers to an x-ray imaging sensor comprising of an array about 1″×1.5″ in area of active pixel cells. In an example embodiment, there may be 900×641 pixel cells in the array. The sensor array may be an analog device. Each pixel cell may store an analog voltage proportional to the intensity of the light hitting the cell during the exposure time.
USB refers to Universal Serial Bus
X-ray tube head refers to a composite assembly comprising a sealed housing, x-ray tube, voltage multiplier boards, and transformer oil.
X-ray tube refers to a vacuum tube component inside the x-ray tube head which, when energized, may produce a cone-shaped beam of x-rays. The tube may comprise a filament, heated cathode, and anode.

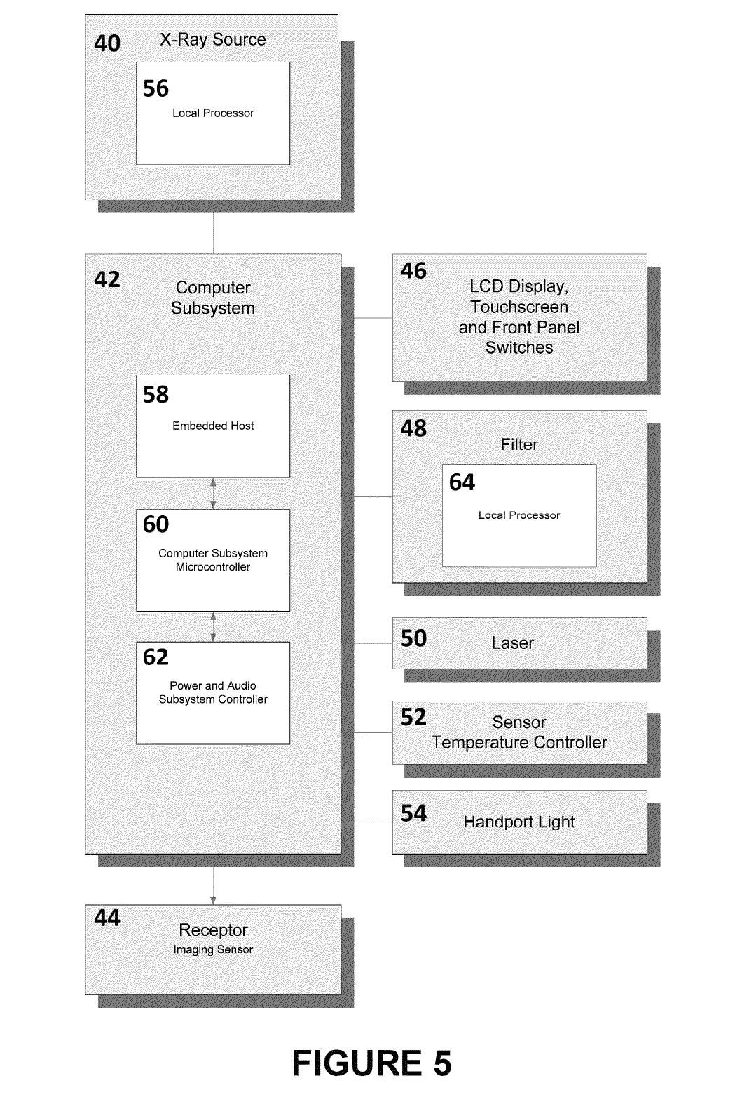

FIG. 5 illustrates an example densitometer functional diagram. The overall densitometer system logical architecture, in an example embodiment as depicted in FIG. 5, may comprise an energy source 40 (e.g., x-ray source), a computer subsystem 42, a receptor (e.g., imaging sensor) 44, a user interface 46, a filter 48, a laser 50, sensor controller (e.g., temperature, etc.) 52, and a light 42, or any appropriate combination thereof. Each of the numbered elements depicted in FIG. 5 may comprise hardware, or may comprise a combination of hardware and software.

In an example embodiment, the energy source 30 may comprise a local processor 44. The local processor 56 may control, manage, or the like, operation and functionality of the energy source 40. In an example embodiment, the computer subsystem 42 may comprise an embedded host 58, a computer subsystem microcontroller 60, a power and audio subsystem controller 62, or the like, or any appropriate combination thereof. A processor, or processors, in the densitometer 12 may perform various functions. For example, instructions for eliminating soft tissue components of a ROI image (described above) may be executed by the local processor 44, a processor of the computer subsystem 42, or any appropriate combination thereof.

The user interface 34 may comprise any appropriate circuitry to perform user interface functions as described herein. For example, the user interface 34 may comprise a display, such as a liquid crystal display (LCD) display, a light emitting diode (LED) display, a plasma display, a cathode ray tube (CRT) display, a touchscreen, indicators, switches, or the like, or any appropriate combination thereof, to perform user interface functions as described herein. The user interface 34, which may comprise a graphical user interface, may be utilized to assist the operator in conducting BMD Tests, Reviewing BMD Test Results, System Tests and System Configuration Tasks and to report exceptions and error conditions to the operator. The user interface 34 (also referred to herein as a graphical user interface—GUI) may comprise a set of menus that may guide the operator through the BMD Test, System Configuration, and System Test tasks. A color touchscreen may be utilized to allow the menus to include color and graphics in their visual design. In addition to the menu system, the GUI may include a status bar to indicate the device state. The status bar may indicate when a printer or USB device is connected, when the device is in Demonstration Mode (the x-ray source is deactivated), and when the device is due for a periodic phantom QC test (e.g., conducted after every 300 BMD Tests). The BMD Test protocol may permit the use of a full touchscreen alphanumeric keyboard for capturing the patient's name. The BMD Test protocol also may request dominant hand information and this may be used to prompt the operator to insert the non-dominant hand when conducting the test. This improves usability of the device and ensured that the correct hand will be used on successive BMD Tests for any given patient.

The densitometer may provide a library of commands to control features of the hardware such as moving the filter arm in and out of position; turning the finger position laser line generator on and off; switching the hand port light on and off; activating, reading and resetting the imaging receptor and arming the x-ray system; accepting user input from the LCD touchscreen. The densitometer hardware platform may expose various features that may be controlled during the course of a BMD test. A library of functions may be provided in the software to exercise those features on demand. The hardware control and monitoring library may be physically separate from the GUI and BMD Test sequencing software.

The densitometer may monitor the state of the System Control Board. The densitometer software may continuously monitor the state of the System Control Board. The hardware monitor layer in the software may broker requests from the hardware control library and status information from the board back to the rest of the application. The hardware monitor layer may be separate from the GUI.

The densitometer may control the sequence of actions required to conduct a BMD Test. The software may be responsible for controlling the sequence of events required to conduct a patient test. It may use the GUI to capture patient information from the operator and to arm the x-ray system for each of the preset technique factors required for imaging the finger. The technique factors may be programmed into the device and, in an example embodiment, may not be changed by the operator.

The densitometer may prompt the operator to activate the x-ray source when required. The software may be responsible for prompting the operator to activate the x-ray beam (which may be achieved through a hardware switch on the front panel), but, in example embodiment, may not activate the beam itself.

The densitometer may calculate and display the results of the BMD test and, optionally, to print the results on an external printer. The software may perform a DXA analysis on the high- and low-energy images captured during the BMD Test. It may present the results (Bone Mineral Density, Bone Mineral Content, t-score and z-score) to the operator via the GUI. The operator may be prompted to print the test result on an externally attached printer.

The densitometer may detect and manage error conditions. During the course of a BMD Test, System Configuration or System Test activity, errors may occur as a result of incorrect operator input or unexpected hardware conditions (such as a stuck filter arm or failed imaging receptor). The system software may be responsible for detecting those conditions and reporting them to the operator via the GUI.

The densitometer may provide various ancillary functions. The software may also provide ancillary functions including data transfer off the device; software upgrades; setting the date and time; self-testing; and manufacturer's features such as in-house calibration, demonstration mode and image storage

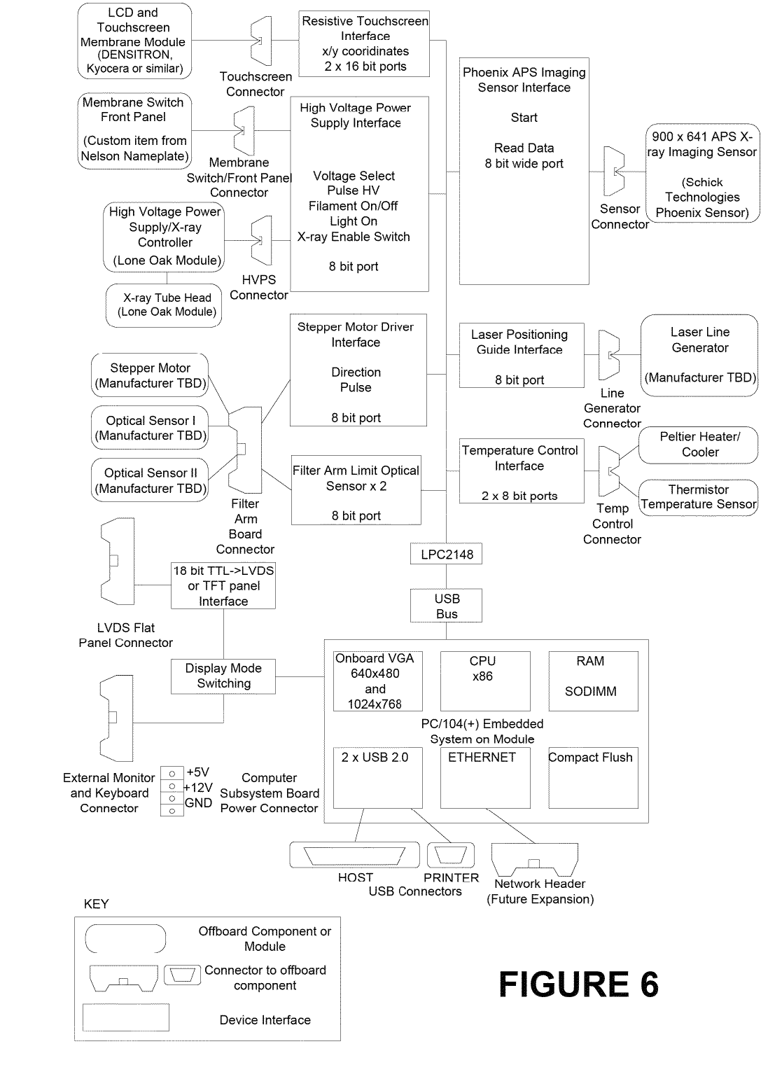

FIG. 6 illustrates an example block diagram of the densitometer hardware environment. In an example configuration, the computer subsystem local processor (e.g., computer subsystem microcontroller 60 depicted in FIG. 5) may be an LPC2148 microcontroller responsible for managing communication with the PC/104 host over the USB bus and dispatching commands to the laser 50, handport light 54, x-ray source 40, receptor 44, temperature control system 52, or any appropriate combination thereof.

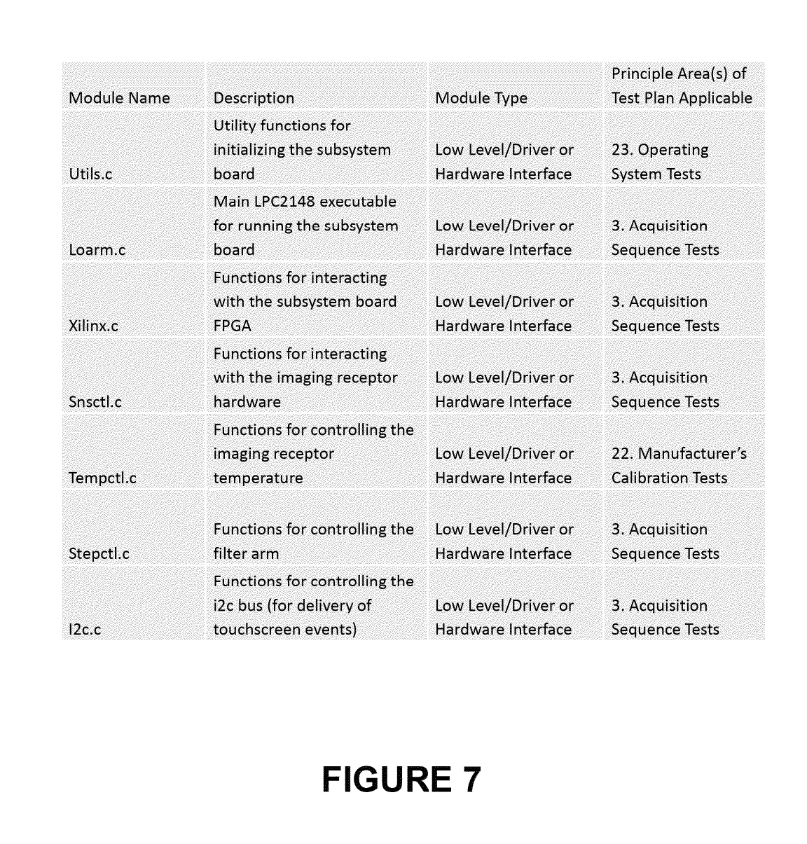

FIG. 7 illustrates an example module table for the densitometer's computer subsystem local processor.

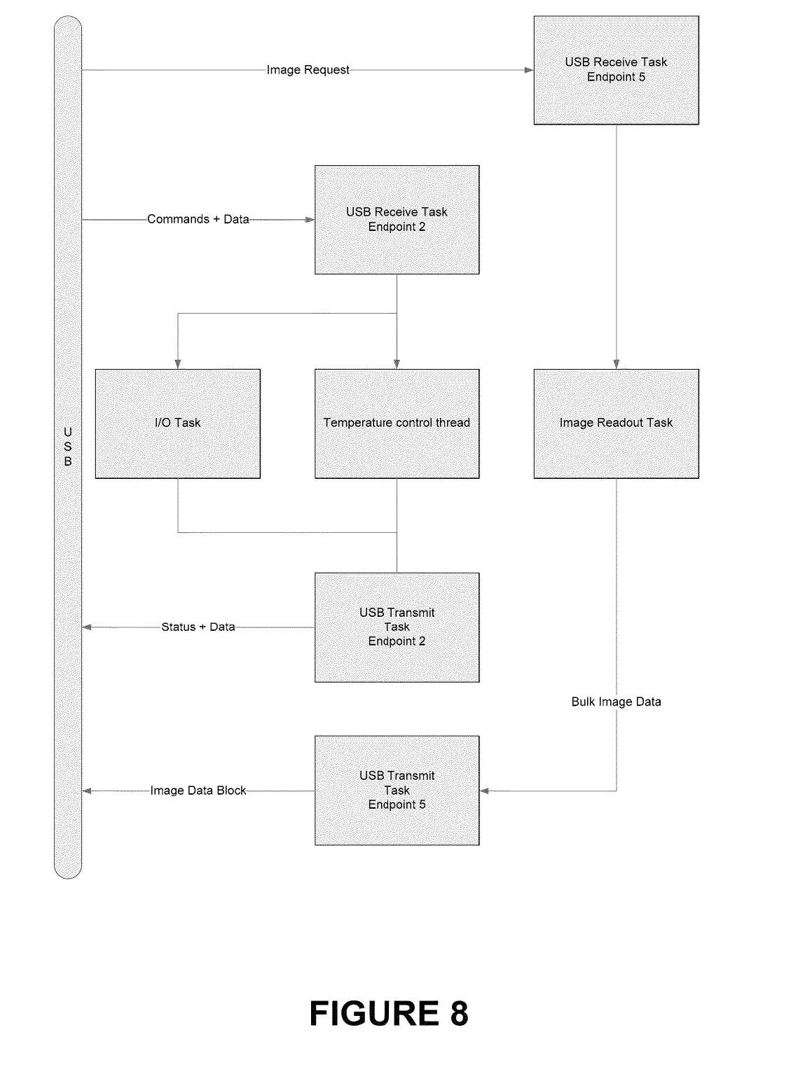

FIG. 8 illustrates an example task model for the densitometer's computer subsystem local processor.

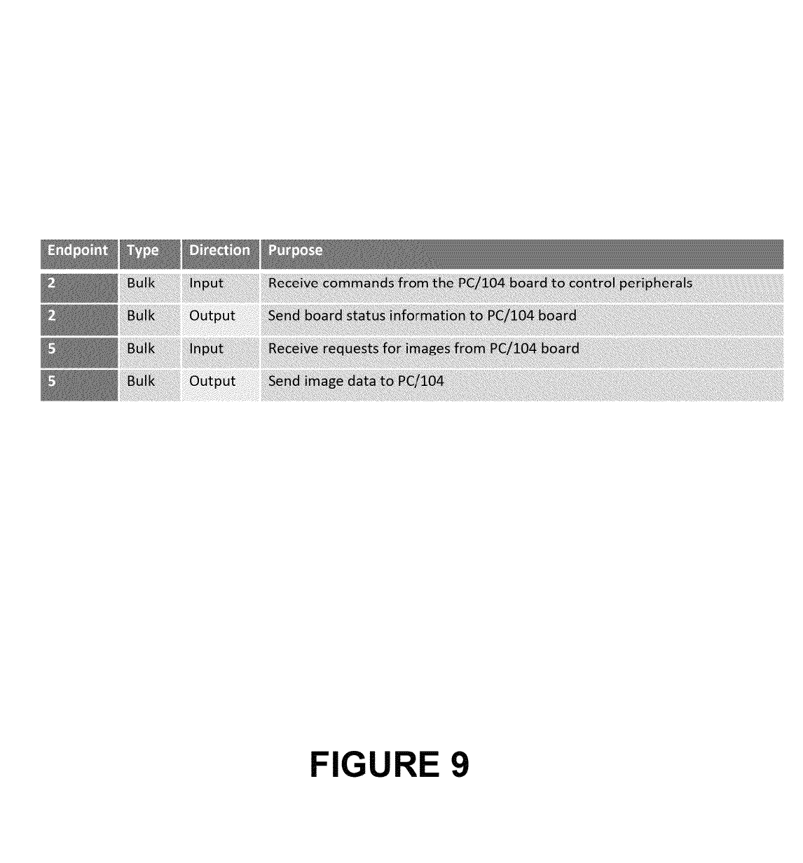

FIG. 9 illustrates an example endpoint definition table for the densitometer's computer subsystem local processor.

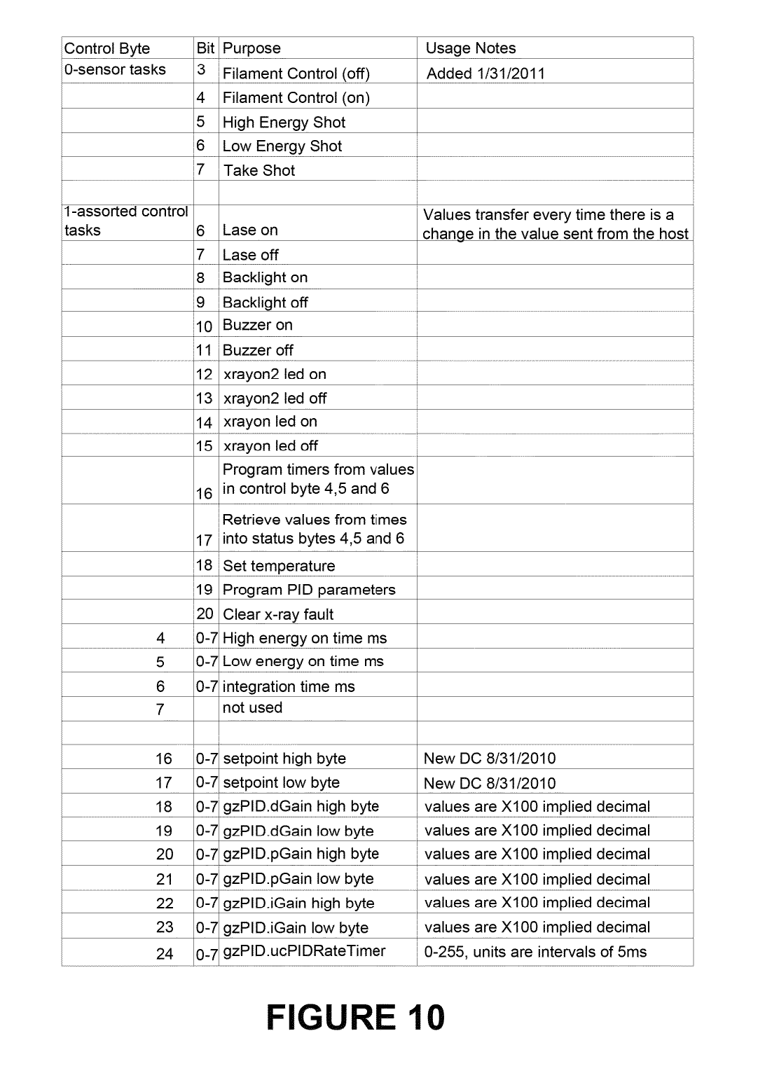

FIG. 10 illustrates example control byte descriptions for the densitometer's computer subsystem local processor. In an example embodiment, a control data block may comprise a block of 64 bytes sent from the PC/104 to the Computer Subsystem Board LPC2148 controller over bulk endpoint 2. FIG. 10 describes example control values (unused values are ignored).

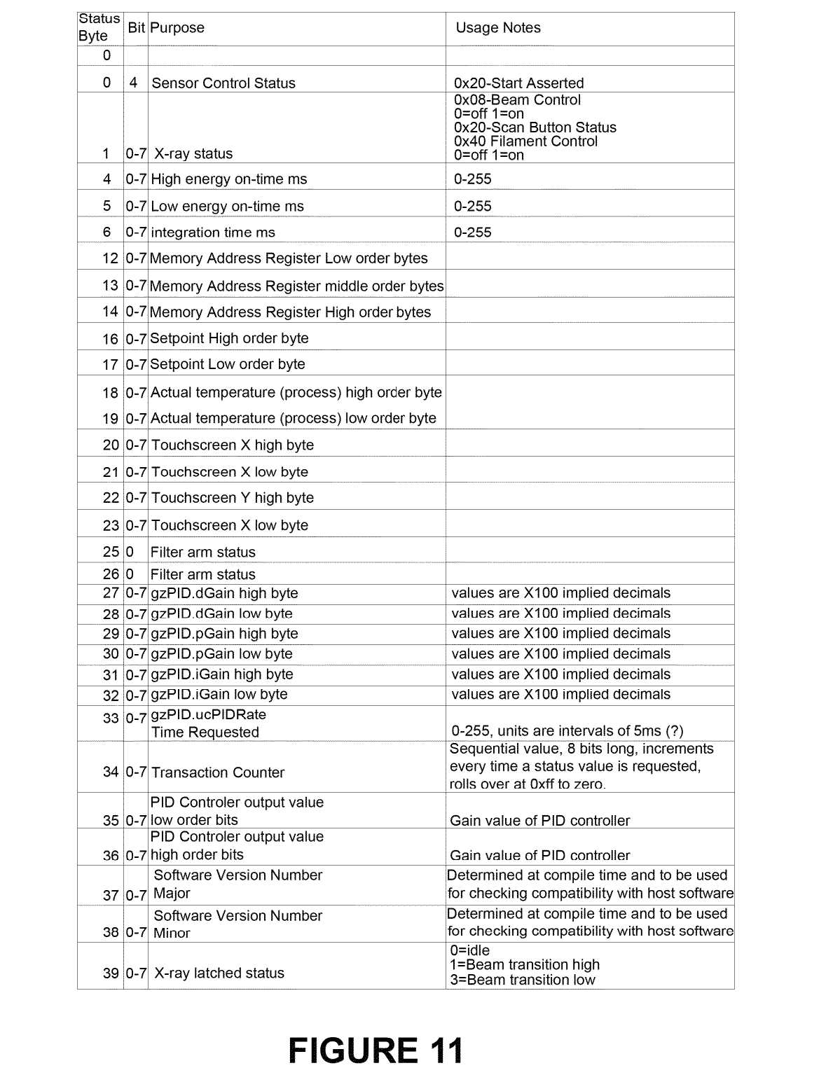

FIG. 11 illustrates example status byte descriptions for the densitometer. In an example embodiment, a status data block may comprise a block of 64 bytes sent from the Computer Subsystem Board LPC2148 controller over bulk endpoint 2. FIG. 11 describes example status values (unused values are ignored).

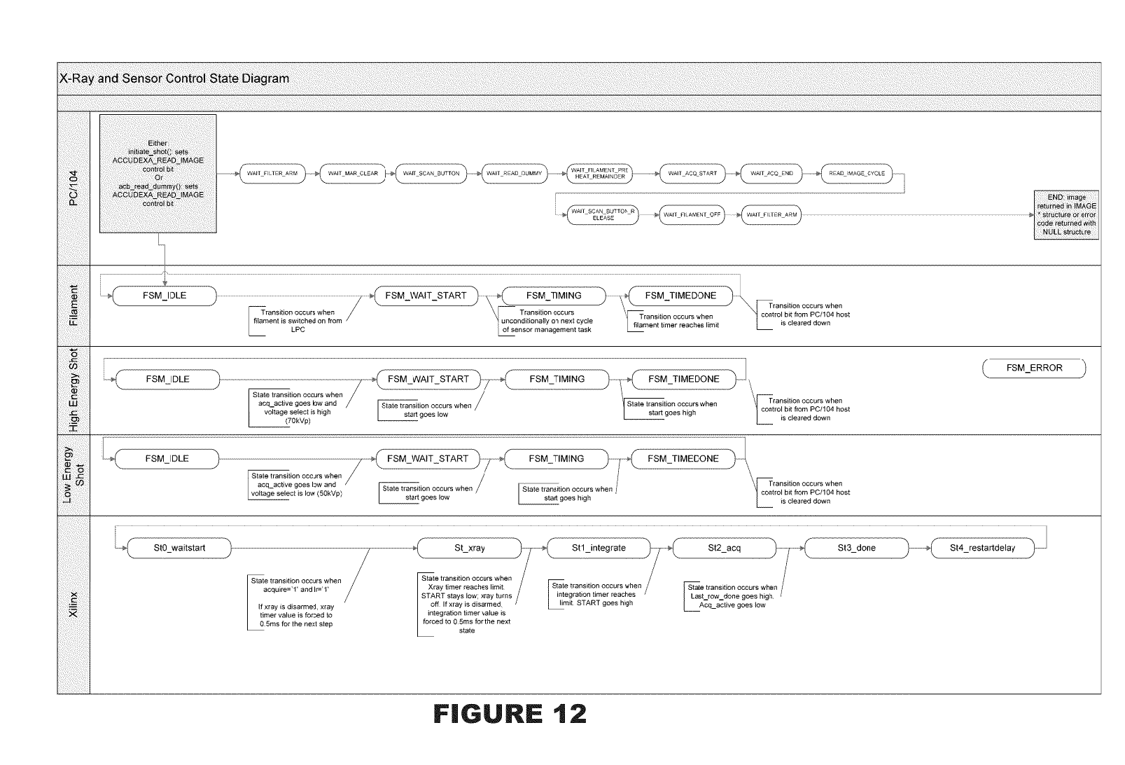

FIG. 12 illustrates an example state transition diagram for the densitometer's x-ray imaging system.

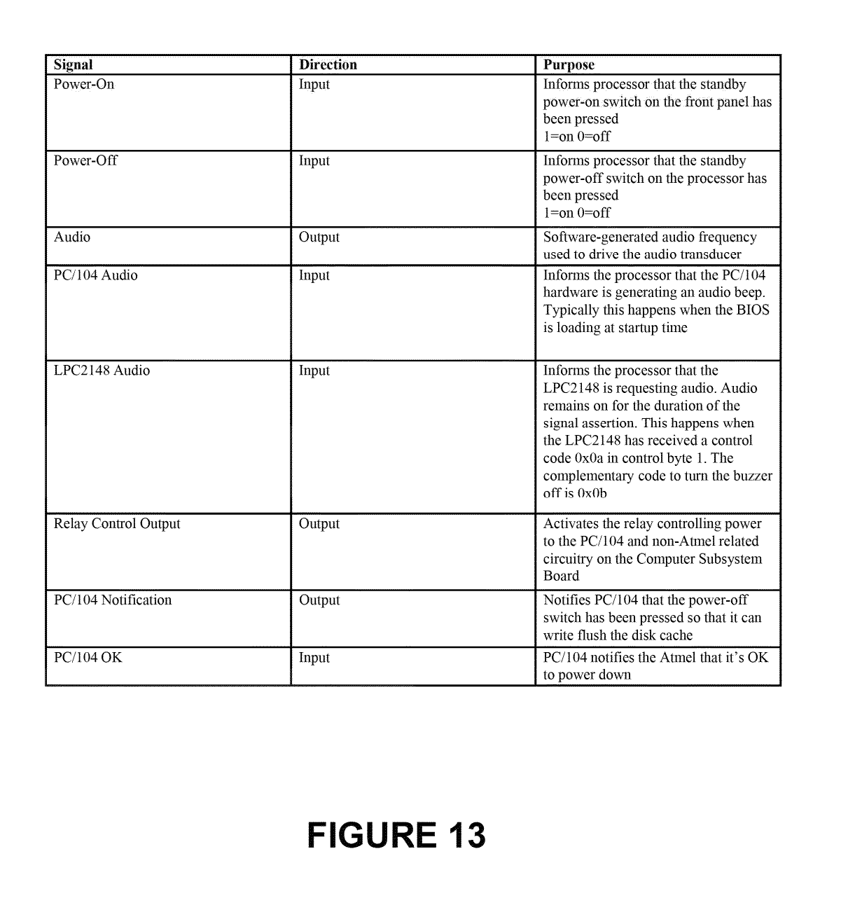

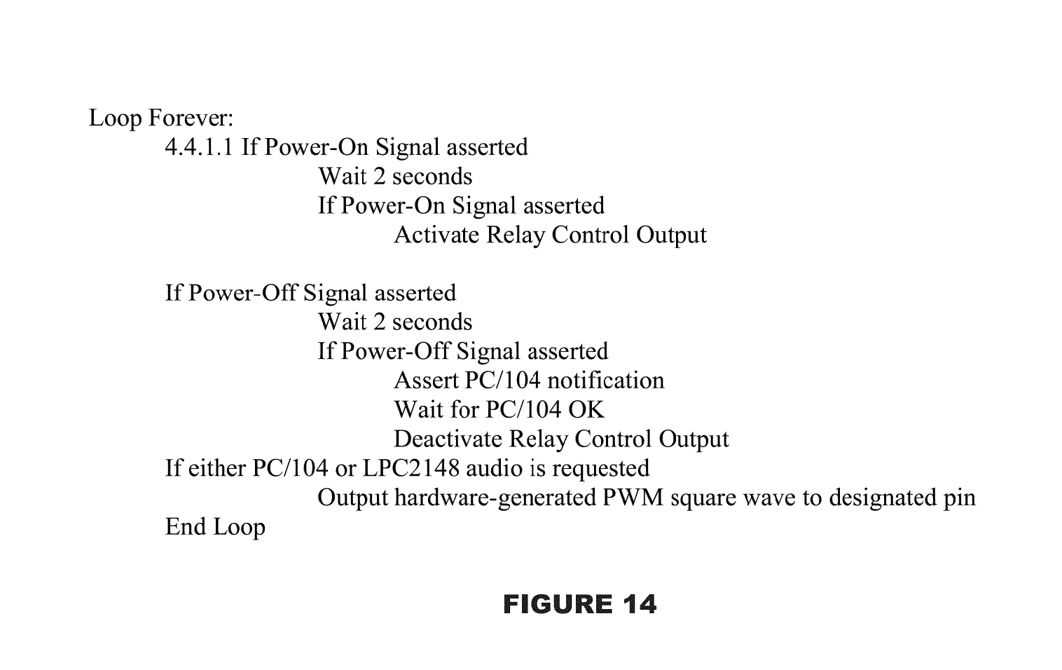

FIG. 13 illustrates example input/output (I/O) descriptions for an example power/audio controller of the densitometer. In an example embodiment, the power/audio controller comprises an Atmel Power/Audio controller, ATTINY microcontroller, located on the Computer Subsystem Board. It may be responsible for managing power-up and power-down to standby mode and creating an AF output to an audio buzzer. A single source file may be used with the Atmel IDE to create a firmware image to load into the processor. The Power/Audio controller may use a single task in a repeated loop to monitor the state of the power-up, power-down and beep signals. The Audio/Power Controller may use a simple single task process such as the example pseudo code depicted in FIG. 14.

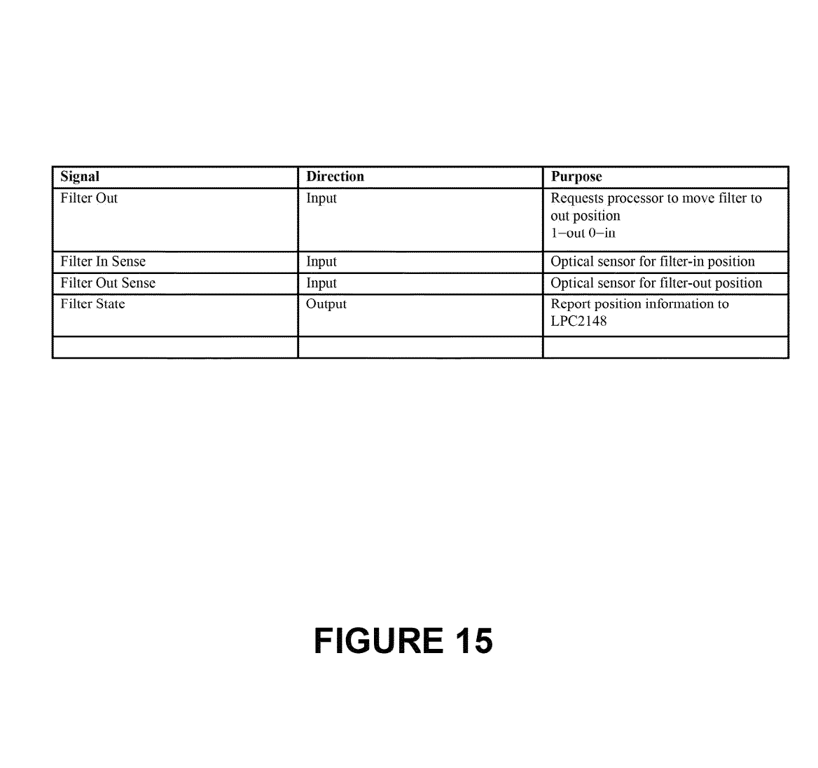

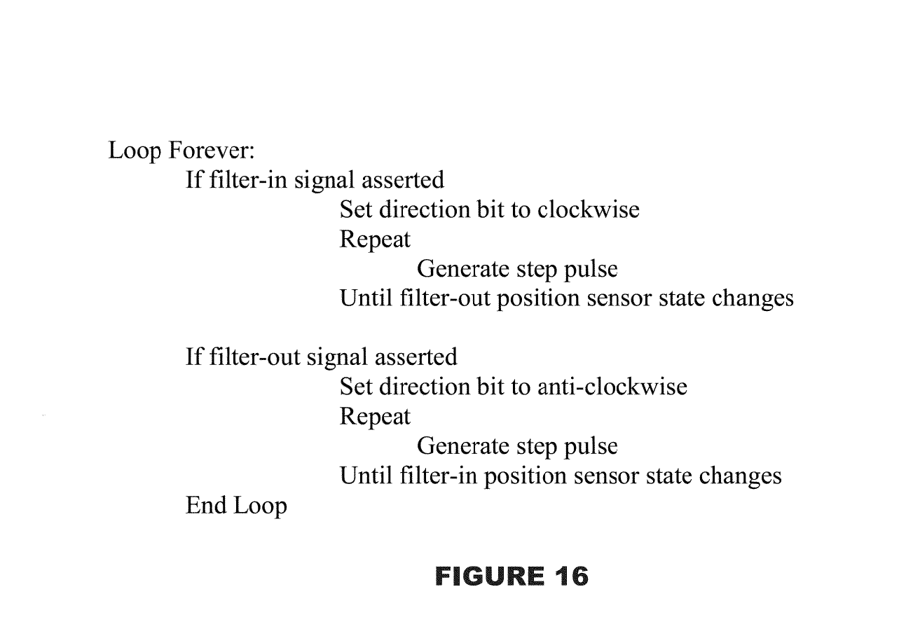

FIG. 15 illustrates example input/output (I/O) descriptions for an example filter arm module of the densitometer. In an example embodiment, the filter arm position controller may comprise a PIC microcontroller located on the Filter Board. It may be responsible for moving the filter arm to its requested position and reporting the filter arm position by reading the optical limit sensors. A single source file may be used with PIC Proton Development Environment to create a firmware image to load into the processor. The filter arm position controller may use a single task in a repeated loop to monitor the state of the filter position request signal, such as depicted in the example pseudo code depicted in FIG. 16.

The x-ray power supply system of the densitometer may comprise an x-ray power supply controller. The x-ray power supply controller may comprise, in an example embodiment, a PIC microcontroller located on the x-ray power supply board. It may be responsible for managing the filament current, reporting the status of the power supply (beam active; error), managing the anode current by monitoring the anode voltage and pulse-width modulating the current drive circuit, and/or providing a secondary/backup timer.

A single source file may be used with PIC Proton Development Environment to create a firmware image to load into the x-ray power supply system processor. The x-ray power supply system processor may use a single task in a repeated loop to monitor the state of the filter position request signal.

When power is applied, the external resonator may be checked to see if it is running at 10 MHz, for example. This may be done by using a watchdog timer at 512 ms, for example, and setting timer zero to 400 ms, for example. If the resonator is running at 5 MHz, for example, the timer may need 800 ms, for example, to time out, but the watchdog time may reset the IC before that occurs. When 12 V power is applied, for example, the 50 KV and 70 KV, for example, dead time may be copied from EEROM to RAM. Then the EEROM location may be incremented by two to allow for wear-leveling of the EEROM. Then the X-Ray on LED may turn on for 1 second. When the LED turns off the X-Ray power supply may be ready for the filament to be turned on. When the filament is turned on it may be set for low power, 30 KHz, for example, with a dead time of 127 for 400 ms, for example. Then the dead time may be changed to 75 for 300 mS, for example. Then the filament drive may be changed to 15 KHz, for example, with the previously-used dead time for 50 KV or 70 KV for 200 ms, for example. After 900 ms, for example, the HV for X-Ray output may be turned on. Note the X-Ray tube filament may need to be on for 1.5 seconds total, for example, to get to the 5 mA setting.

When power is applied to the filament, and if there is a short circuit in the filament circuit, power may be turned off to the filament and the fault LED may be turned on. Turning off the filament may clear the fault. After the third try, for example, the fault LED may be turned on and the X-Ray LED and X-Ray output may be flashing. Turning off the filament may not clear this fault. The filament may need to be turned on for 100 mS and off for 500 ms to clear this fault condition. If the filament is on for more than 30 seconds, for example, the filament timer may cause the filament to be turned off and the fault LED may be turned on. To clear this fault, the filament may be turned off. When the HV is turned on the X-Ray head may emit X-Rays. 6 ms, for example, after the HV is turned on, the mA control may start adjusting the dead time for the filament drive so the X-Ray tube current is 5 mAs, for example. When the HV is on the KV is checked to see if the 50 KV is between 45 KV & 60 KV, for example, and the 70 KV is between 65 KV & 85 KV, for example. The mAs may be checked to see if it is between 4.5 mA & 5.5 mA, for example. If the KV or mAs is out or range when the HV is on, the HV may not be turned off. When the HV is turned off and the KV or mAs are out of range this may generate a fault. The fault LED may be turned on and the X-Ray LED and X-Ray output may be flashing. To clear this fault the filament may need to be turned on for 100 ms, for example, and off for 500 ms, for example. If the HV is on for more than 200 ms, for example, the HV may be turned off. This fault may be cleared when the filament is turned off. When the HV then the filament is turned off the dead time for the filament drive may be stored in EEROM if different from the last exposure. The X-Ray power supply may be ready for the next exposure. If the HV is turned off and the filament is left on there may be 10 seconds, for example, to change the KV setting before a fault is generated. When changing the KV setting, the HV may be turned on after 200 ms, for example. There may be have 30 seconds, for example, after the KV setting changes before a fault is generated. The fault may be cleared by turning off the filament.

Host software of the densitometer may be responsible for managing the GUI and interaction with the end user, controlling high- and medium level device features, performing BMD Tests and printing results, performing QC Phantom Tests, utility functions such as setting the date and time, and/or transferring test results to other media.

PC/104 module dependencies may be managed using, for example, the application master Makefile.

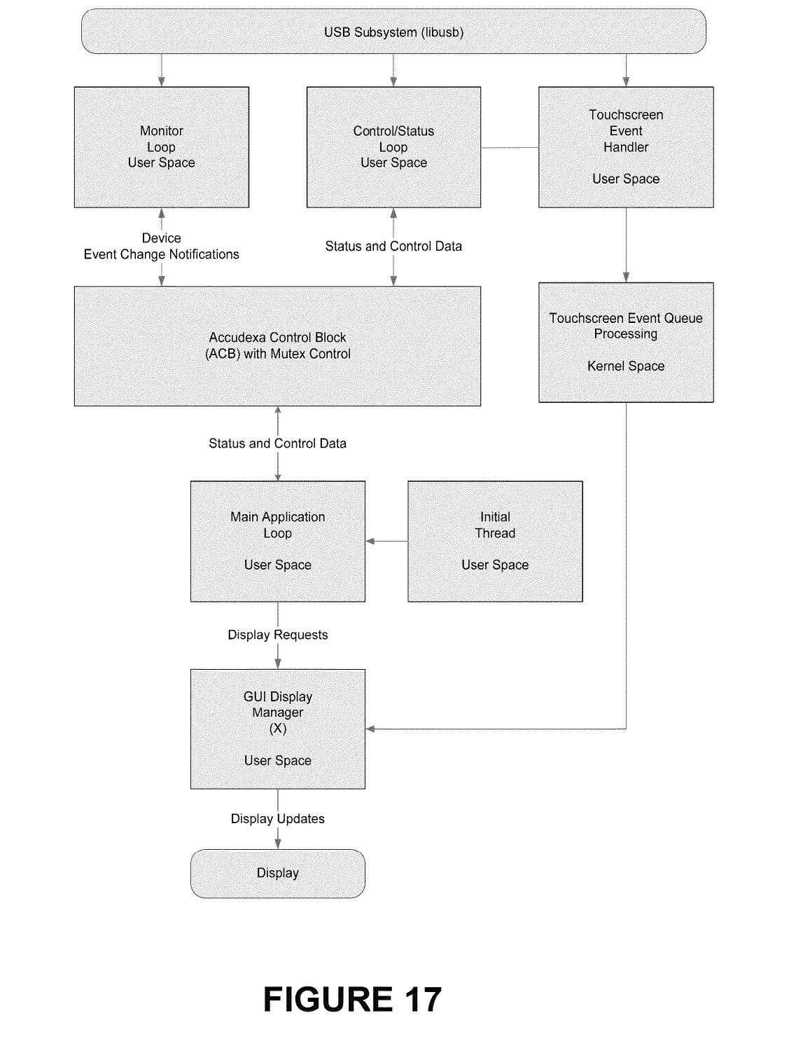

FIG. 17 illustrates an example task model for the densitometer. Application processing tasks—I/O, GUI, Device Monitoring—may be allocated to individual threads. The application design may use Posix threads to establish a multi-threaded environment. Data sharing may occur between the control/status loop thread (function control_status_loop( ) in usblpclib.c) and the dependent threads via a thread-safe data area called the AccuDEXA® Control Block. Access to the ACB is under mutex control. To gain access to the ACB, a thread must call the function lock_acb( ), read or write the data and then call unlock_acb( ) to unlock the mutex.

The initial program thread may be created when the densitometer program is invoked by the startup process (script run-gt.sh, invoked by startx during rc initialization). It may be responsible for creating all dependent threads required by the application, initializing the GUI using the function create_AccuDEXA®_widgets( ) and initializing data structures. If the program has been invoked manually in maintenance mode, the initializing thread may handle the display of the maintenance mode menus via the function menu_loop( ). Otherwise, for normal production use, once the dependent threads are running, the initializing thread may remain idle until the gtk main loop terminates before terminating itself.

This thread, once created, may immediately invoke the gtk main loop function gtk_main_loop( ). This loop function may be responsible for handling all GUI events to and from the display and touchscreen. It may invoke application functions via the callback system as needed. Because the GUI is event-based, all functionality may be invoked from the callback mechanism within GTK2. This may necessitate the use of a state model to track the current state of the application between callbacks. Two functions, such as for example, —get_gui_state( ) and set_gui_state( ) may allow the GUI state to be set using values from the following list taken from gtkgui.h:

/* These defines are the states for the UI */
#define MAIN_MENU 0
#define PATIENT_ID 1
#define PATIENT_NAME 2
#define PATIENT_AGE 3
#define PATIENT_GENDER 4
#define PATIENT_ETHNICITY 5
#define PATIENT_DOMINANT_HAND 6
#define PATIENT_SUMMARY 7
#define EDIT_PATIENT_ID 8
#define EDIT_PATIENT_NAME 9
#define EDIT_PATIENT_AGE 10
#define EDIT_PATIENT_GENDER 11
#define EDIT_PATIENT_ETHNICITY 12
#define EDIT_PATIENT_DOMINANT_HAND 13
#define POSITION_FINGER 14
#define ENGAGE_XRAY 15
#define ANALYSIS_WINDOW 16
#define CALCULATING_BMD 17
#define PRINT_DECISION 18
#define SYSTEM_CHECK_MENU 19
#define DIAGNOSTICS 20
#define UPGRADE_MENU 21
#define UPGRADING 22
#define TOUCHSCREEN_CAL_WINDOW 23
#define CONFIGURE_SYSTEM 24
#define SET_DATE 25
#define SET_TIME 26
#define BURN_IN_TEST 27
#define SYSTEM_STARTUP 28
#define PHANTOM_QC_TEST_START 28
#define PHANTOM_QC_POSITION_PHANTOM 29
#define PHANTOM_QC_CALCULATING_BMD 30
#define PHANTOM_QC_SHOW_RESULTS 31
#define PHANTOM_QC_REPEAT 32
#define PATIENT_FILE_OPTIONS_MENU 33
#define PATIENT_LOG_LIST_SCREEN 34
#define TRANSFER_START_DATE 35
#define TRANSFER_END_DATE 36
#define TRANSFER_DELETE_AFTER_COPY 37
#define TRANSFER_SUMMARY 38
#define TRANSFER_IN_PROCESS 39
#define TRANSFER_COMPLETE 40
#define PRINT_MULTIPLE_COPIES 41
#define REPRINT_LIST 42

A combination of the current GUI state and the identity of the button or widget that originated the triggering event may determine the next state of the application. The main callback functions may be defined in the module gtkgui.c, and may be as follows.
static void abort_button_click_cb (GtkWidget *widget, gpointer data)—invoked when the imaging sequence abort button is clicked. The status of the abort flag is altered by this function and it is monitored by the function initiate_shot( ) in module usblpclib.c
static void reprint_button_click_cb (GtkWidget* widget, gpointer data)—invokes functionality to review previous test results for reprinting.
static void system_checkbutton_click_cb (GtkWidget *widget, gpointer data)—invoked when system check menu and submenu buttons are clicked. Determines which button was pressed and performs associated functionality
static void board_status_button_click_cb (GtkWidget *widget, gpointer data)—only invoked when in maintenance mode and any of the board test feature buttons are invoked
static void phantom_button_click_cb (GtkWidget *widget, gpointer data)—invoked when the phantom calibration test button is clicked. Performs phantom calibration procedure
static void bmd_button_click_cb (GtkWidget *widget, gpointer data)—invoked when the BMD Test button is clicked. Starts the BMD Test by checking that the BMD test is not locked out because a phantom test has failed, or that there is an x-ray system error. If correct entry conditions are met, sets the GUI state to PATIENT_ID which begins the test protocol.
static void temperature_setpoint_cb (GtkSpinButton *spinbutton, gpointer user_data)—invoked when temperature setpoint changes occur in maintenance mode. Transfers the settings from the temperature control adjustment to the temperature demand values in the ACB.
static void print_report(GtkButton *button, gpointer data)—invoked when either a printer test or a BMD Test Report are to be printed. Invokes function print_button_clicked( ) in module new_print.c.
static void print_qc_report_cb (GtkButton *button, gpointer data)—sets up conditions for printing a QC test report and invokes function print_button_clicked( ) in module new_print.c.
static void upgrade_menu_button_click_cb(GtkWidget* widget, gpointer data)—if monitor loop thread has detected that an upgrade package is present, the upgrade procedure is invoked by setting the GUI state to UPGRADE_MENU. This callback provides all functionality associated with the software upgrade process.
static void phantom_button_cb(GtkWidget* widget, gpointer data)—invoked when the Phantom Test button is clicked. Performs all functions required to conduct a Phantom QC Test.

When the main application code is invoked in production mode or maintenance mode, it may attempt to connect to the computer subsystem board over the USB. The function AccuDEXA®_connect( ) in module usblpclb.c may be invoked, which in turn may invoke the function enumerate_AccuDEXA®( ). This may open the usb device and establish the bulk endpoints for communication with the board. It may pass a valid device handle back to AccuDEXA®_connect( ), or an error state if the board could not be enumerated.

Assuming the board connection was established, AccuDEXA®_connect( ) may create a new thread using the function acb_control_status_loop( ), the first responsibility of which is to allocate a new AccuDEXA® Control Block (ACB) if none currently exists. Having done so, it may immediate lock the ACB to begin a cycle of data transfer in and out of the ACB.

If an image request is in progress, acb_control_status_loop( ) may attempt to read and unpack a full image frame from bulk endpoint 5. The frame may comprise (900×641×2) bytes. Since the pixel data is 12 bits wide but the USB bulk transfer system word boundaries are 16 bits, the computer subsystem board may pack successive pixel values so that the remaining 4 bits are not wasted but are used for pixel value transfers. This may improve image transfer speed by about 25%. Acb_control_status_loop( ) must therefore unpack the 12 bit values into 16 bit words as it receives the data.

To handle status transfers from the computer subsystem board, the control status loop may attempt to read 64 bytes from the board. Short reads or timeouts result in an error. Assuming the read is successful, the values may be unpacked, formatted and transferred to specific areas of the ACB. Example code may comprise the following:

/* Copy status block into control block */

memcpy(acb->status, ibuf, BULK_BUF_SIZE);
//int w;
//printf(“ibuf:\n”);
//for(w=0;w<35;w++)
// printf(“%d: %x\n”, w, ibuf[w]);
acb->memory_address_register=(65536*ibuf[14]) +

(256*ibuf[13]) + ibuf[12];

acb->heartbeat=ibuf[0]&0x01;
acb->start_status=acb->status[0]&0x20;
acb->temperature_setpoint=256*ibuf[16]+ibuf[17];
acb->temperature_process=256*ibuf[18]+ibuf[19];
acb->output=256*ibuf[35]+ibuf[36]; /* 11/19/2010 DC Control

output ×100 */

if(acb->output>32768)

acb->output=acb->output-65535;

/* 11/17/2010 DC Capture PID parameters */
acb->pgain=256*ibuf[29]+ibuf[30];
acb->igain=256*ibuf[31]+ibuf[32];
acb->dgain=256*ibuf[27]+ibuf[28];
acb->pid_timer=ibuf[33];
//printf(“pgain=%d igain=%d dgain=%d pid timer=%d\n”,

acb->pgain, acb->igain, acb->dgain, acb->pid_timer);

/* 11/9/2010 DC Capture sensor temperature at start of
integration */
if(!(acb->status[0] & 0x20)) {

acb->sensor_integration_temp=acb->temperature_process;
printf(“Temp from board: %d\n”, acb->temperature_process);

}
acb->touchscreen_x=(16*ibuf[21]+ibuf[22])/16;
acb->touchscreen_y=(16*ibuf[23]+ibuf[24])/16;
acb->high_on_time=acb->status[4];
acb->low_on_time=acb->status[5];
acb->integration_time=acb->status[6];
acb->filter_arm_status=acb->status[26];
acb->scan_button_status=acb->status[1] & 0x20;
acb->status[32]=‘\0’;
acb->xray_latched_status=acb->status[39];
if(acb->xray_latched_status!=prior_latched_status)

printf(“\n\nxray latched status changed from %u to

%u\n\n”, prior_latched_status, acb->xray_latched_status);

prior_latched_status=acb->xray_latched_status;
acb->software_version_major=acb->status[37];
acb->software_version_minor=acb->status[38];

The control status loop may then send out any pending command data to the subsystem. To send a command to the board, the program may set up the value in the 64 byte array acb->control, and then may set the flag acb->pending by calling the function set_pending( ). If the control status loop sees the pending flag is set, it may transmit the 64 byte control block to the computer subsystem board. It then may clear the pending flag. The transmitting function may stall by calling the function wait_pending( ). Using a combination of set_pending( ) and wait_pending( ), application functions may queue commands and block until the command has been sent. In practice, application functions may call the functions set_AccuDEXA®_control_bit( ) and clear_AccuDEXA®_control_bit( ) which OR-in the requested values to the control bits and then make the call to set_pending( ). The application function may directly call wait_pending( ) and blocks until the pending flag in the ACB is clear. Example: code to move the filter arm may comprise the following.

void filter_in( ) {

printf(“+filter_in( )\n”);
lock_acb(“filter_in”);
clear_control_block(acb);
unlock_acb(“filter_in”);
set_accuDEXA ®_control_bit(acb,
ACCUDEXA ®_FILTER_IN);
wait_pending(acb);
clear_accuDEXA ®_control_bit(acb,
ACCUDEXA ®_FILTER_IN);
wait_pending(acb);
lock_acb(“filter_in”);
clear_control_block(acb);
unlock_acb(“filter_in”);
printf(”−filter_in( )\n”);

}

Finally, acb_control_status_loop( ) may unlock the ACB through a call to unlock_acb( ). It may yield to any other competing GTK2 threads through a call to g_yield_thread( ), and sleeps for 15 ms which allows time for other threads to access the ACB data. The loop then repeats until the system is shut down.

The monitor loop task may be responsible for monitoring device change activity in the USB subsystem. It may look for the insertion or removal of USB disks and printers. If a valid drive has been inserted and mounted to the system mount point /mnt/tmp, the code may look to see whether various files are present. If the file .show-diag is present, the GUI may be instructed to display the Diagnostic Menu option when appropriate, along with the Set Demo Mode/Cancel Demo Mode options. If any file with a suffix of .tar.gz is present, the monitor loop task may attempt to process that file as an upgrade by applying various validations to it before setting the GUI state to UPGRADE_MENU and displaying the upgrade screen. If the Configure System menu is being displayed, the monitor loop may update the date and time display. If a printer has been plugged in, the monitor loop may be responsible for finding an appropriate CUPS printer driver, activating it and notifying the user. If the printer is unsupported, the monitor loop may generate an appropriate error message and displays it.

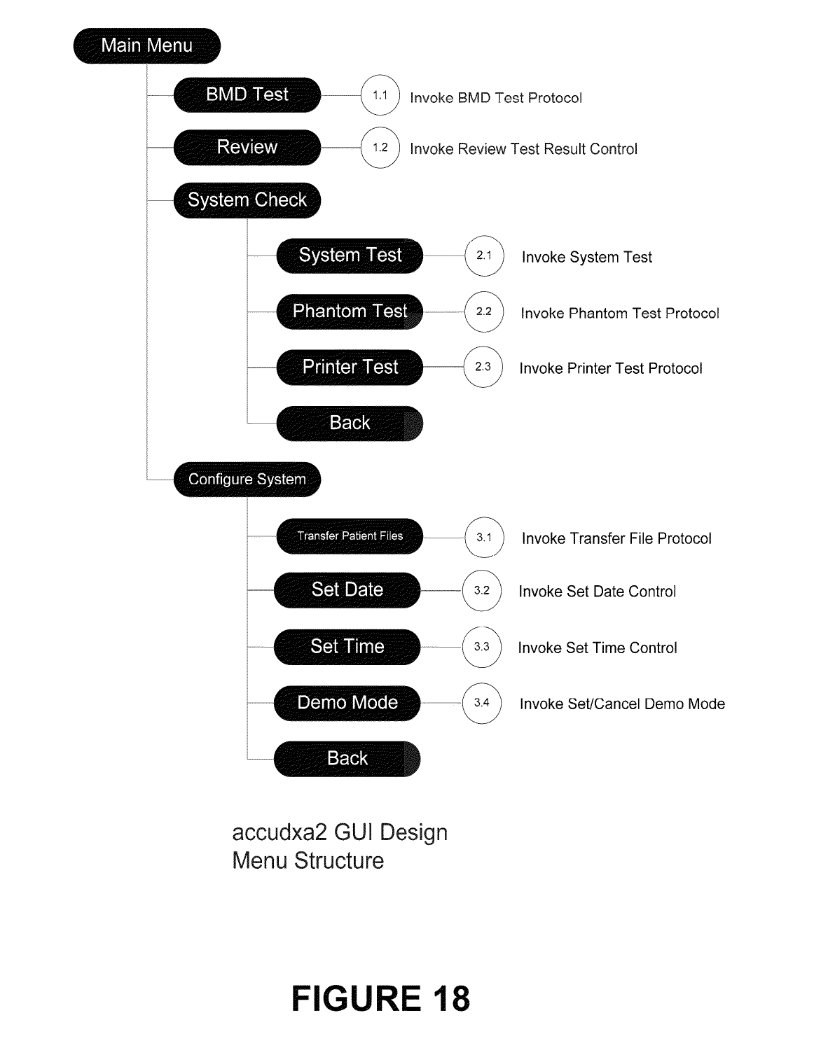

FIG. 18 depicts an example graphical user interface (GUI) menu structure for the densitometer.

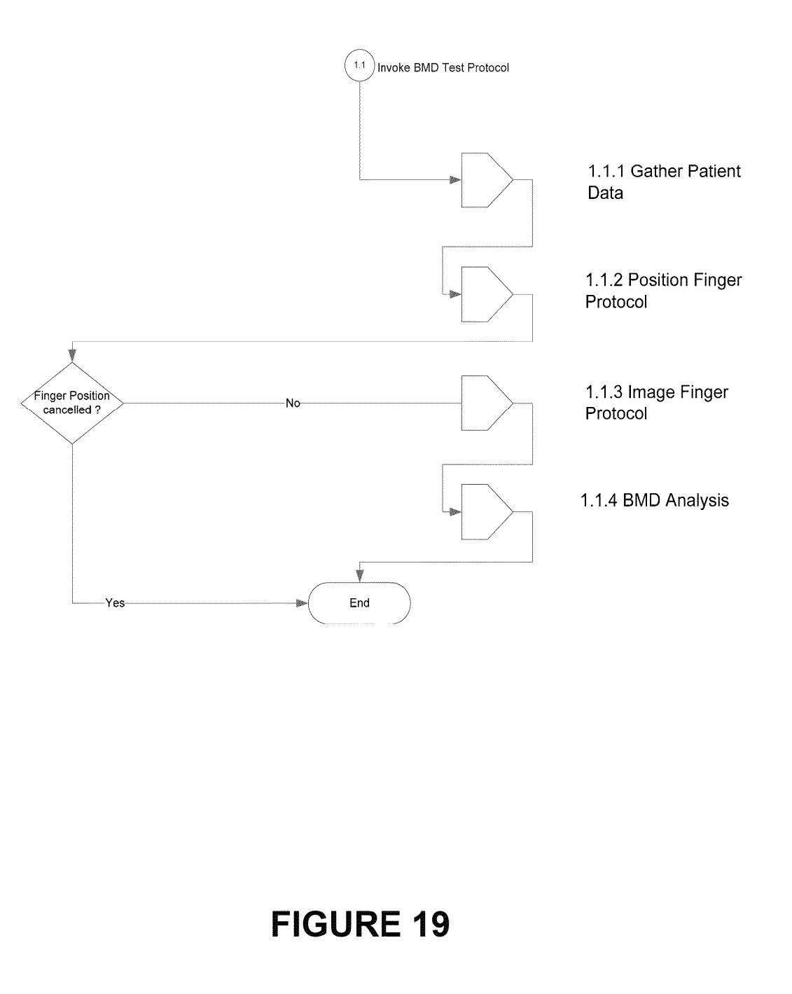

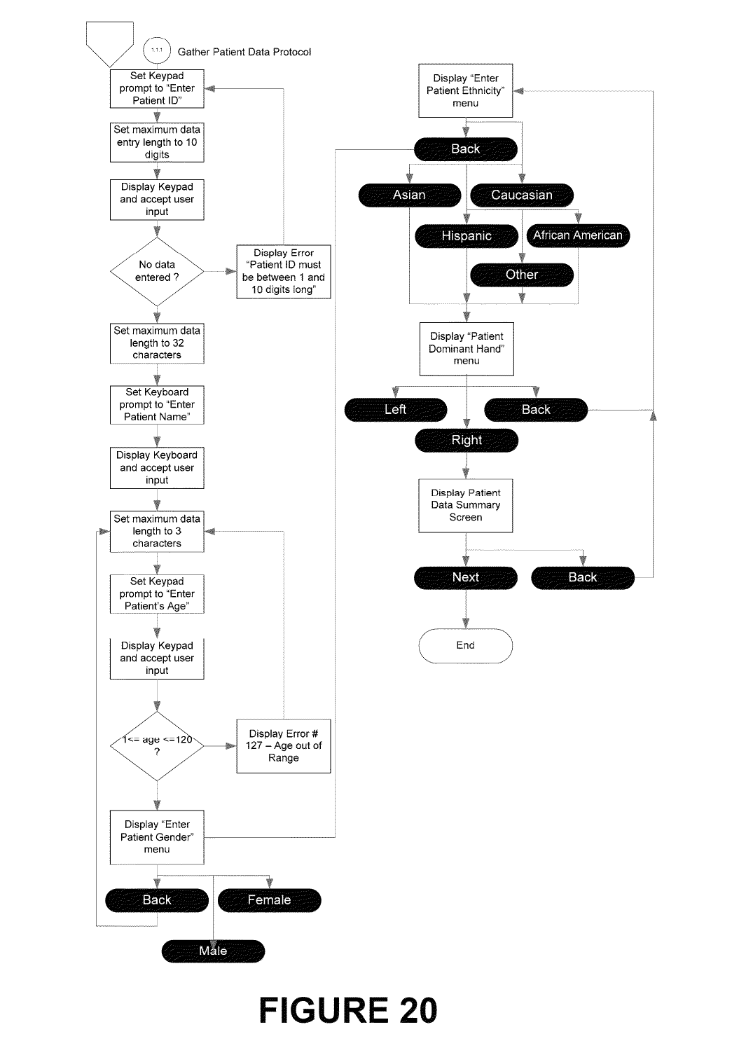

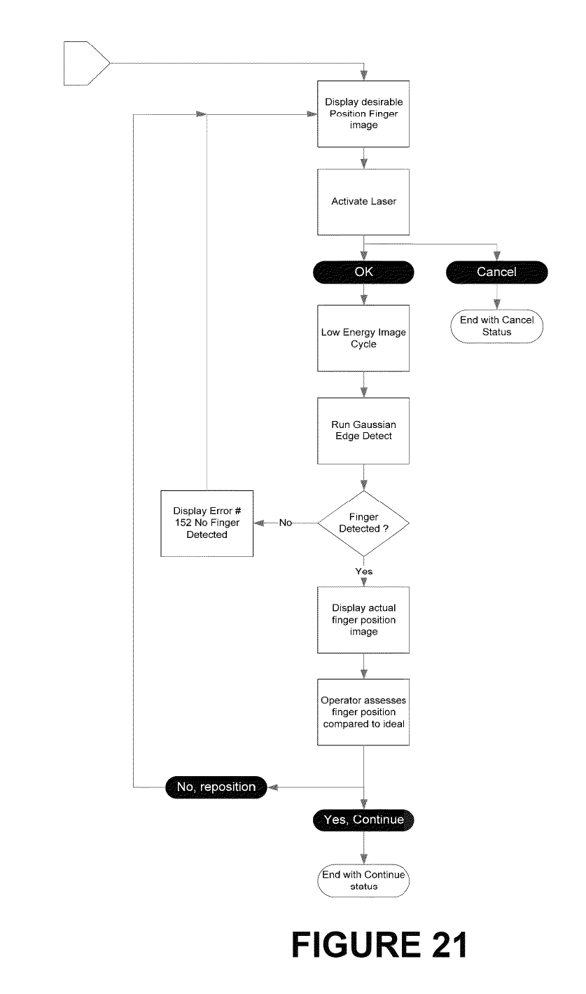

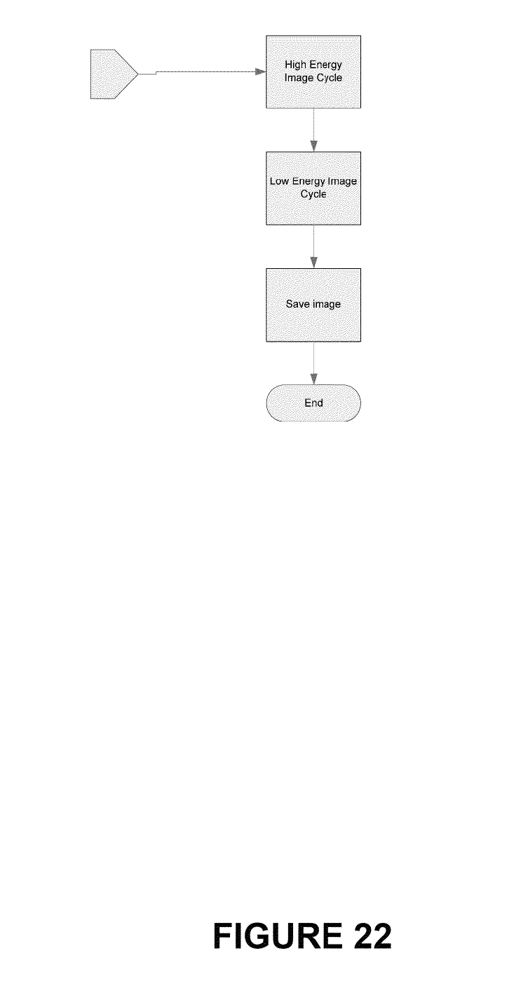

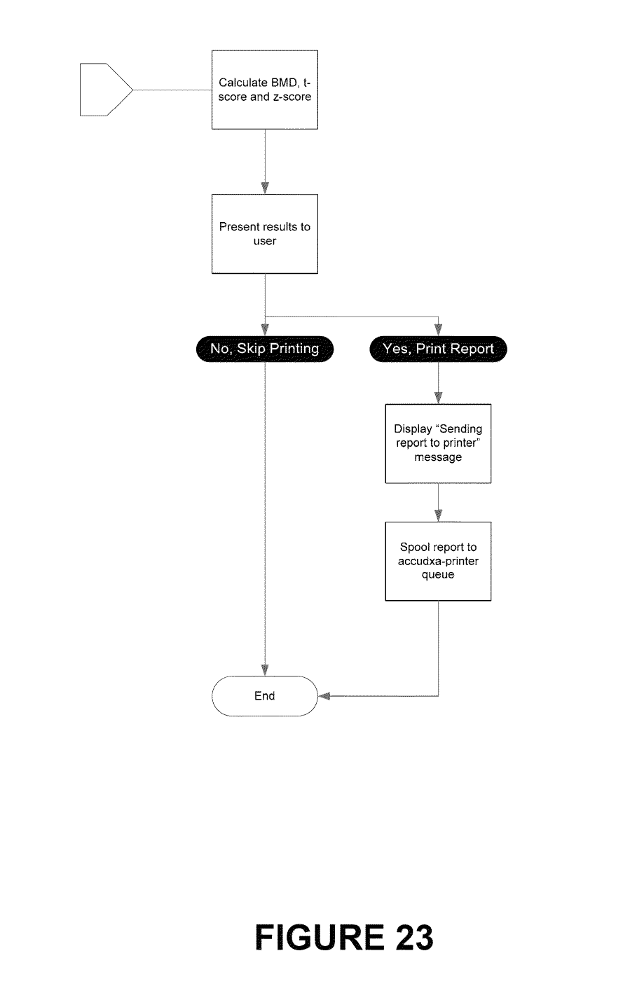

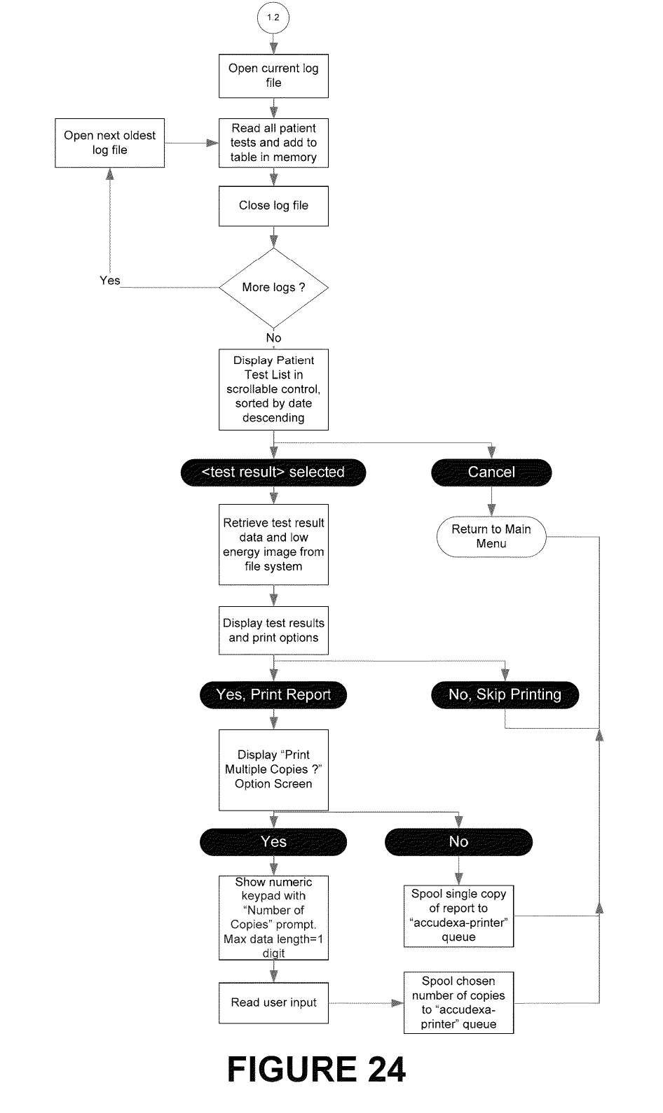

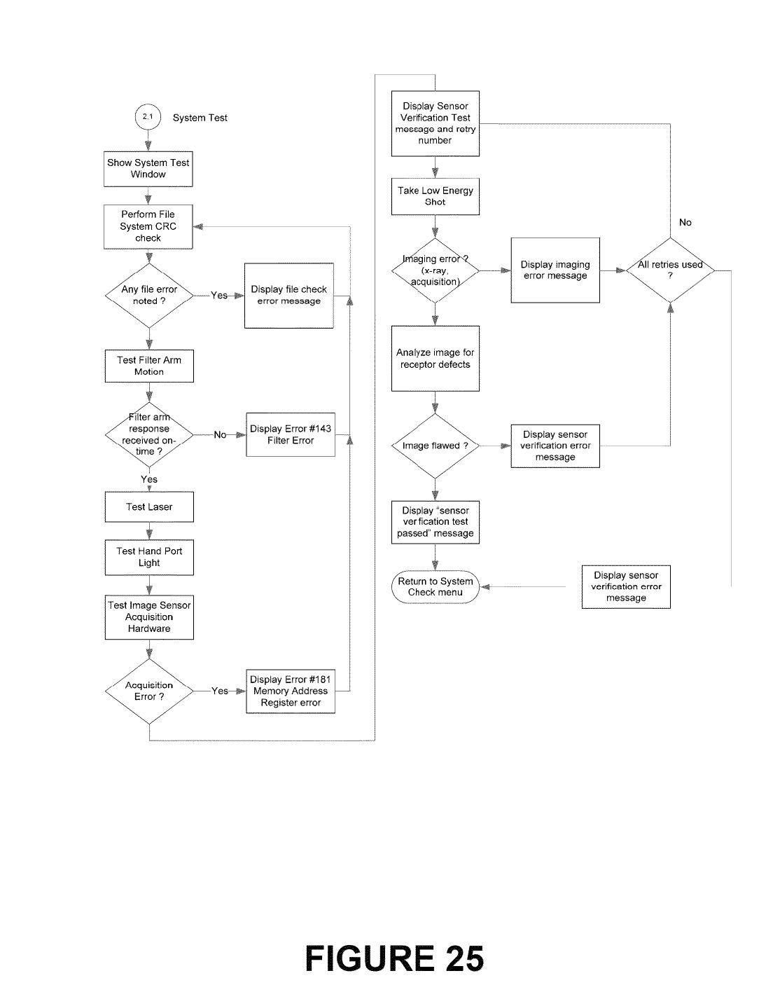

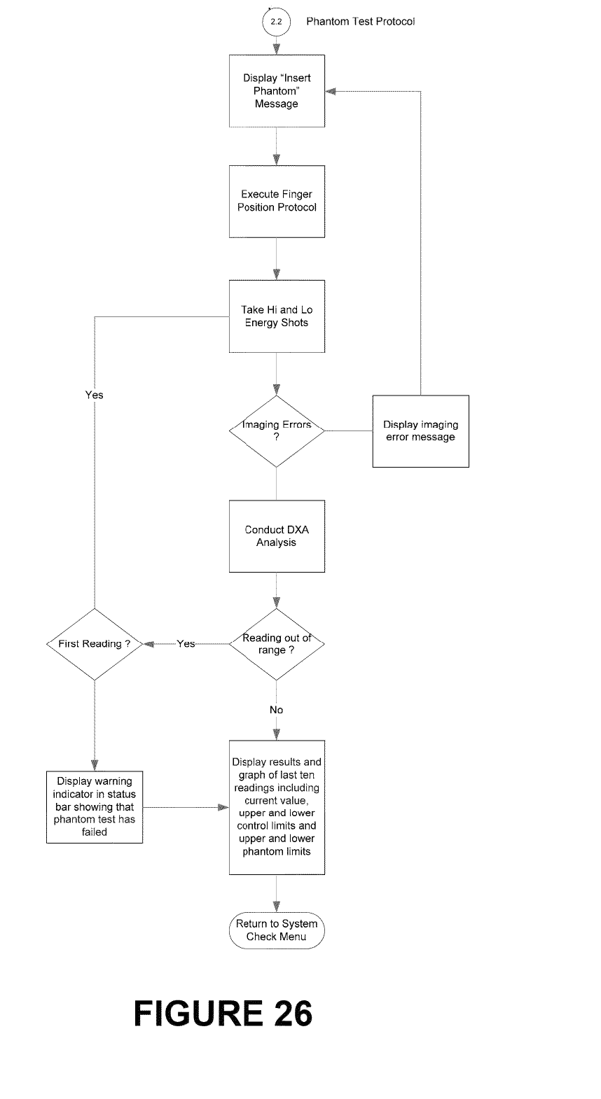

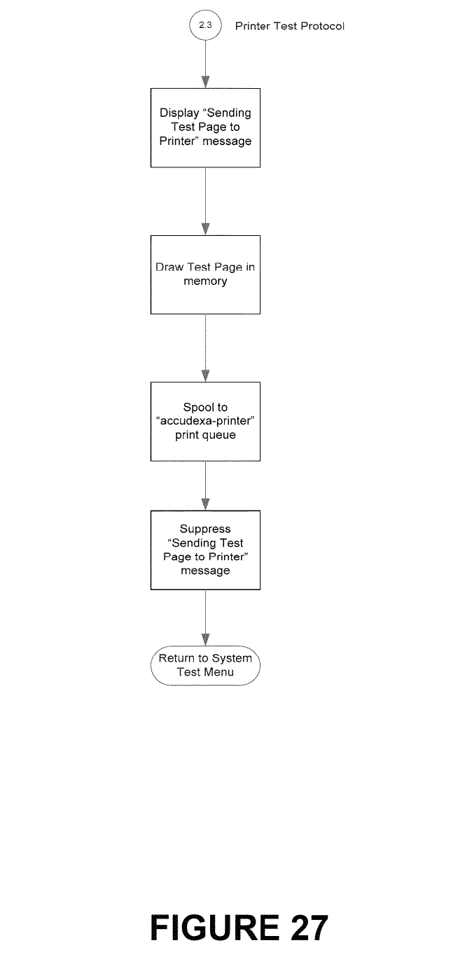

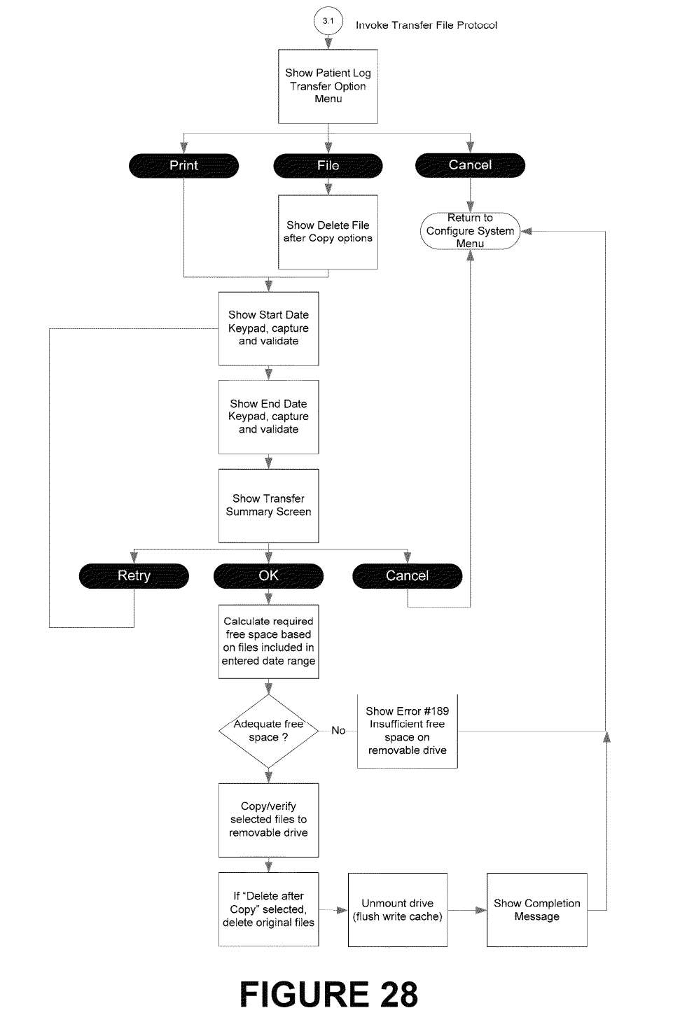

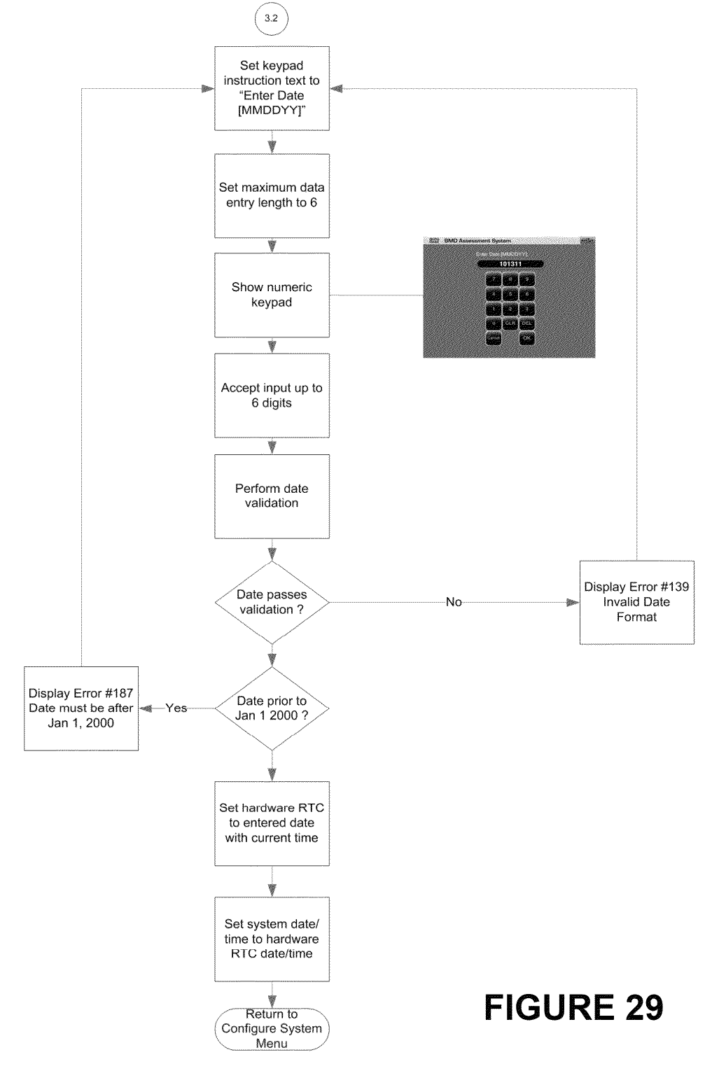

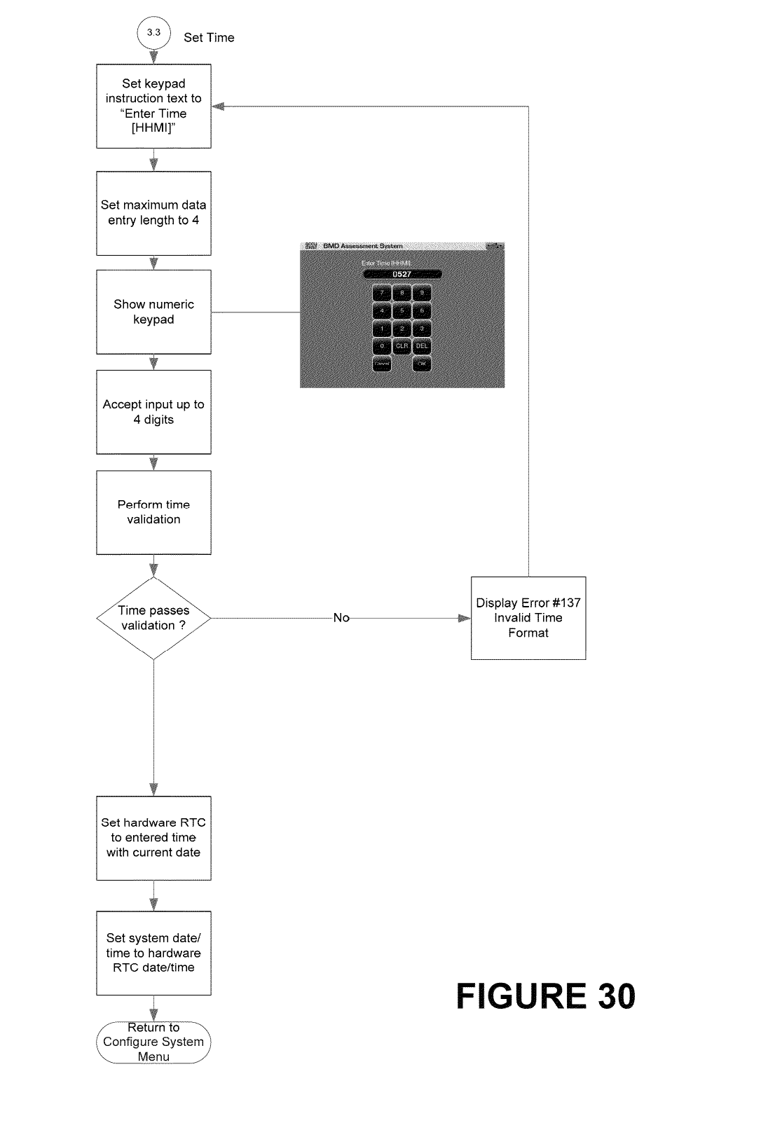

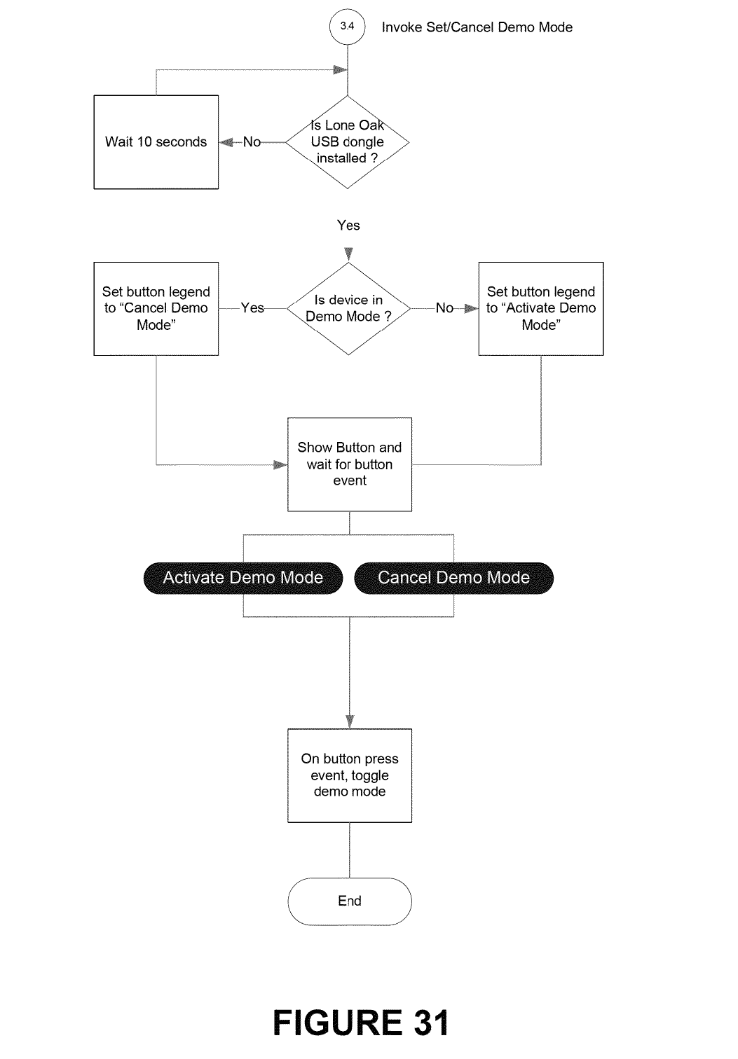

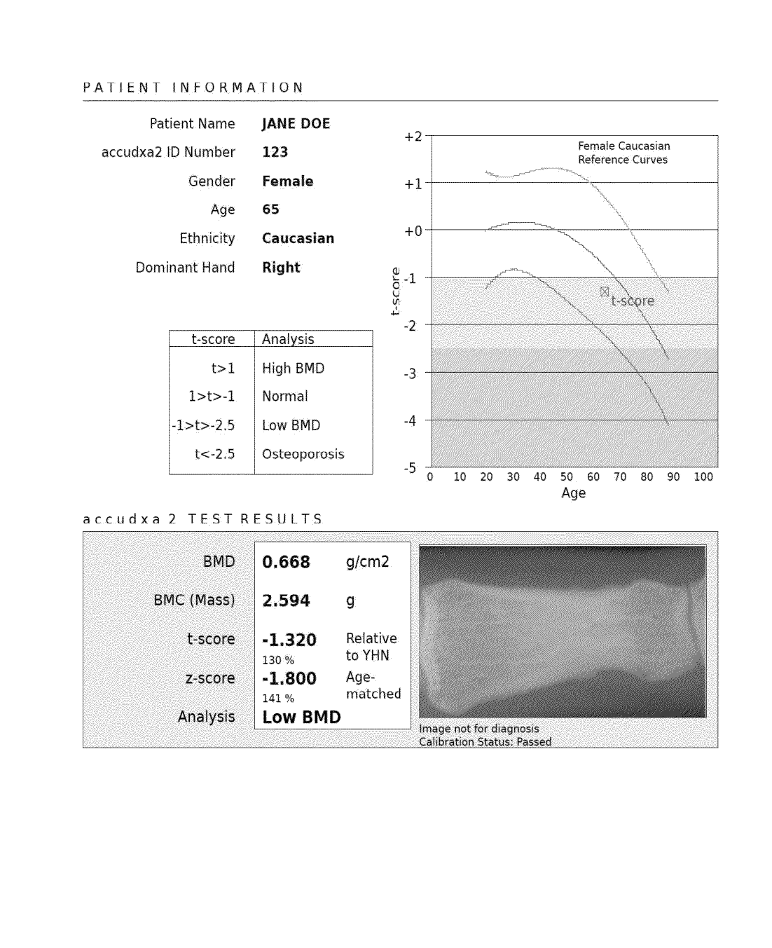

FIG. 19 through FIG. 31, depict an example application flow diagram for user functions of the densitometer.

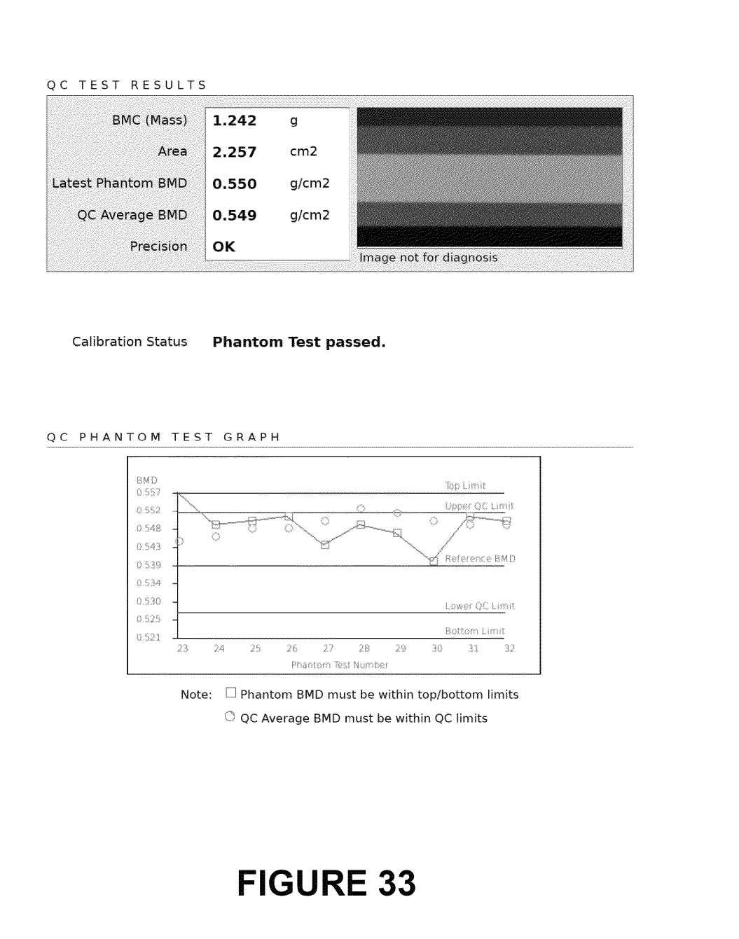

FIG. 32 depicts an example BMD test report. FIG. 33 depicts an example QC Phantom Test Report.

In various embodiments, the densitometer may be upgradeable. Upgrades may be made available via software distributed via the web. Upgrade packages may be distributed as compressed tar files (.tar.gz). A user may place an upgrade package on a USB thumb drive which may be formatted as an NTFS file system, for example, allowing it to be recognized by Windows PCs, Macs and Linux computers, or the like. In an example scenario, a USB drive bearing an upgrade package may be inserted into a USB slot on the densitometer. After the drive has been recognized and mounted to /mnt/tmp, for example, the monitor loop thread may detect the presence of the package on the drive.

Package names may be generated by the packaging program so that the release and build numbers may be encoded into the filename. An example would be AccuDEXA®-2.00a-build-277-i386.tar.gz which is build 277 of the release 2.00a software.

In order to prevent downgrades of the system software, the upgrade function may first determine the current version of the running software by making a call to, for example, the function get_build_number( ). The package build number may be parsed from the package name and compared to the currently running build. If the upgrade package is older than the current release, the filename may be added to the package ignore list. This may prevent the upgrade system from being retriggered by packages that have already been dismissed from installation. If there is more than one package present, the packages may be processed in alphabetical order, meaning that older releases may be processed before newer ones. If the package has been found to be acceptable, and the GUI state is currently at, for example, MAIN_MENU (preventing spurious triggering of the upgrade during a BMD test or other operation), the GUI state may be set to UPGRADE_MENU and the upgrade menu is displayed. Control of the upgrade process may then pass to the application main loop.

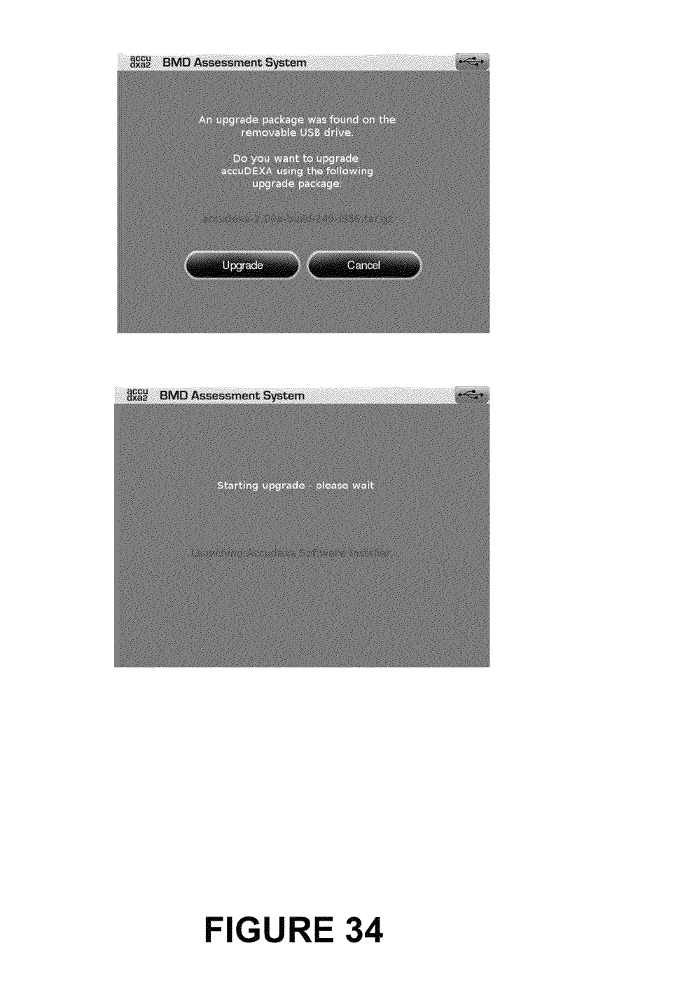

FIG. 34 depicts an example upgrade menu that may be made available to a user. The upgrade menu may have options for Upgrade or Cancel, and may contain information about the package to be installed. If the operator selects Cancel, the package may be added to the ignore list and the GUI state returns to MAIN_MENU. If the operator selects “Upgrade”, the callback upgrade_menu_button_click_cb (GtkWidget* widget, gpointer data) may be invoked and the Upgrade Status screen may be displayed. The upgrade process may perform a CRC check on the existing densitometer .ini file which may contain device-specific settings. This file may be retained without error during the software upgrade process. The package may then be moved from /mnt/tmp to /home/AccuDEXA®, for example. The status of the file move may be checked and the upgrade may be terminated if there was a problem. At this point the installer may be launched. The install program may be a two-phase process. The first phase may unpack and check the package, and ensure that there is sufficient disk space to complete the upgrade. It also may ensure that all required directories are in place. The results of the first phase of the install process may be written to a log file. If the first phase was successful, an installer hook may be placed in /home/AccuDEXA®, for example. The installer hook may be a shell script that will invoke the second phase of the install process. The software may then set the handport to a flashing mode, indicating that the machine is in the middle of an upgrade and should not be interrupted. It then may call for a reboot of the machine which may cause the master executable to terminate and the operating system to reboot.

A densitometer startup script may check for the existence of the installer hook and, if present, may invoke it. The installer hook may execute the second phase of the install process before the executable is started up and this may allow it to replace the executable in its entirety if the package requires it to be upgraded. The second phase of the installer may restore the original AccuDEXA®.ini crc value, for example, to the master CRC file and then may copy all files from the staging directory to the home directory. It may then invoke the postinstall script that was packaged with the upgrade package. The postinstall script may be used to move various files to their final locations and to perform other upgrade actions specific to the package. The staging files may be removed and a call may be made to udevadm, for example, to reload any USB device rule files that may have been upgraded. The final status of the upgrade may be written to the installer status file. The master executable may be restarted. During startup it may look for the installer status file and if present may display the contents of the file to the user. In this manner, the status of the upgraded may be communicated to the end user. Since the upgraded software also may include a new CRC file with CRC values for the upgraded files, the CRC validity of the upgrade may be automatically checked by the startup CRC check and any errors reported to the user during that process.

The following sections provide guidance to a user of the densitometer. In an example embodiment, a system comprising the densitometer may comprise a QC test finger phantom, an AC line cord, a replacement sensor cover(s), and a CD containing a user guide. Spare finger phantoms may be available as replacements for lost items. Hygienic disposable sensor covers may be replaced in the field. Test results may be printed on an optional external printer. In an example, the densitometer may support a range of printer, such as, for example, inkjet printers, laser printers, dot matrix printers, thermal printers, or the like. The densitometer may comprise USB ports for connecting peripherals (e.g., a printer) and for transferring data (e.g., patient test records to an optional removable USB thumb drive). A durable plastic case may be made available for storing and transporting the densitometer, the optional printer, other accessories, etc.

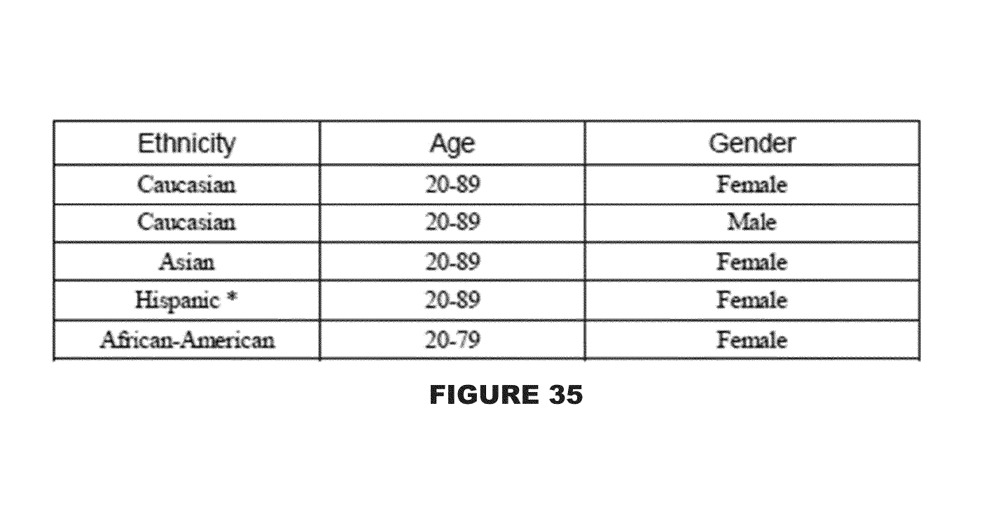

The densitometer functions as a dual-energy X-ray device that can estimate the BMD of the region of the third finger of the non-dominant hand, which may be used as a relative indicator of bone density in other parts of the body. The densitometer may determine an individual's relative BMD status by calculating a t-score and z-score. This calculation may be performed automatically by the densitometer and may be viewed on-screen and/or printed out at the conclusion of an exam. The t-score or z-score may be used as one factor, in conjunction with other clinical indicators, to diagnose osteoporosis and other bone disorders. T-scores and z-scores may be computed if a normative database of other individuals with the same age, gender, and ethnicity of the patient is available. When the matching reference database is unavailable, a patient's BMD may still be used to compare with an initial baseline value. An example normative database is depicted in FIG. 35.

Low bone mineral density at the finger may be predictive of generalized fracture in the elderly as measurements made at axial sites. All bone mineral density measurements may be used in conjunction with other risk factors in determining fracture risk. Other clinical measurements such as blood pressure and cholesterol indicate risk of stroke and myocardial infarction, for example. Similarly, evidence of osteoporosis may indicate risk of fracture.

BMD is an appropriate parameter by which to monitor changes in bone mineral density effected by drug therapy or aging. Results of BMD tests taken on a patient over a period of time may be compared with the reported densitometer precision (repeatability). To determine whether a significant change in BMD has occurred, the percentage change in results over time according to the following formula may be calculated.

% change=(BMD previous exam−BMD current exam)/BMD previous exam*100%

The information below may aid in a determination of the statistical significance of the BMD test result changes. (In an example embodiment, a greater-than-1.8% difference in BMD results may indicate consequential change.)

| Percentage Change in BMD | Level of Statistical Significance |
|---|---|
| 2.77% | 95% |
| 2.33% | 90% |
| 1.84% | 85% |

(These values are based on the densitometer's precision of 1%.)

Below normal bone density may be associated with a variety of bone conditions or disorders. Some of the more common conditions associated with below normal bone density include:
Premenopausal oophorectomy
Spontaneous menopause or estrogen deficiency conditions
Treatment-related osteopenia; when the diagnosis of osteopenia is suggested or
established by other means (such as X-ray; during long-term immobilization)
Endocrinopathies associated with osteopenia; for post-gastrectomy and other
malabsorption states leading to osteopenia; during long-term corticosteroid therapy
Chronic renal disease, particularly in childhood or adolescence

In addition to the above, BMD values may be used to monitor longitudinal changes, as with treatment programs for osteoporosis.

Contraindications may include:
A deformity that prevents a patient's non-dominant hand from being properly positioned.
Orthopedic hardware in the middle finger of the non-dominant hand.
Previous fracture of the middle finger of the non-dominant hand.
Pregnancy. (Although the radiation exposure from the densitometer BMD test may be 1/150,000 of a chest X-ray, any radiation exposure during pregnancy should be deemed medically necessary by a physician.)

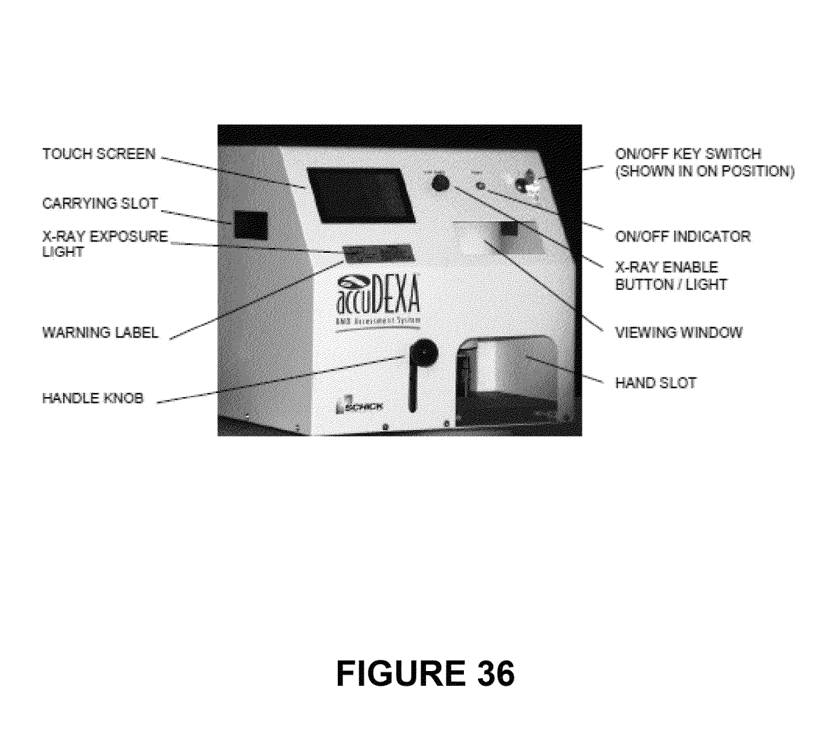

FIG. 36 is an example depiction of a front view of an example embodiment of the densitometer.

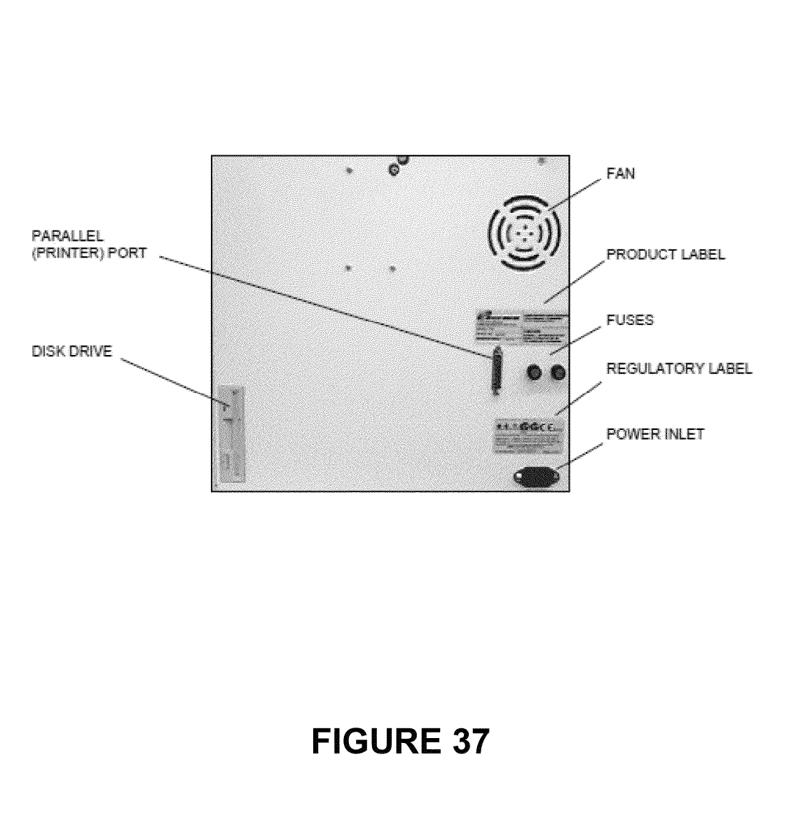

FIG. 37 is an depiction of a back view of an example embodiment of the densitometer.

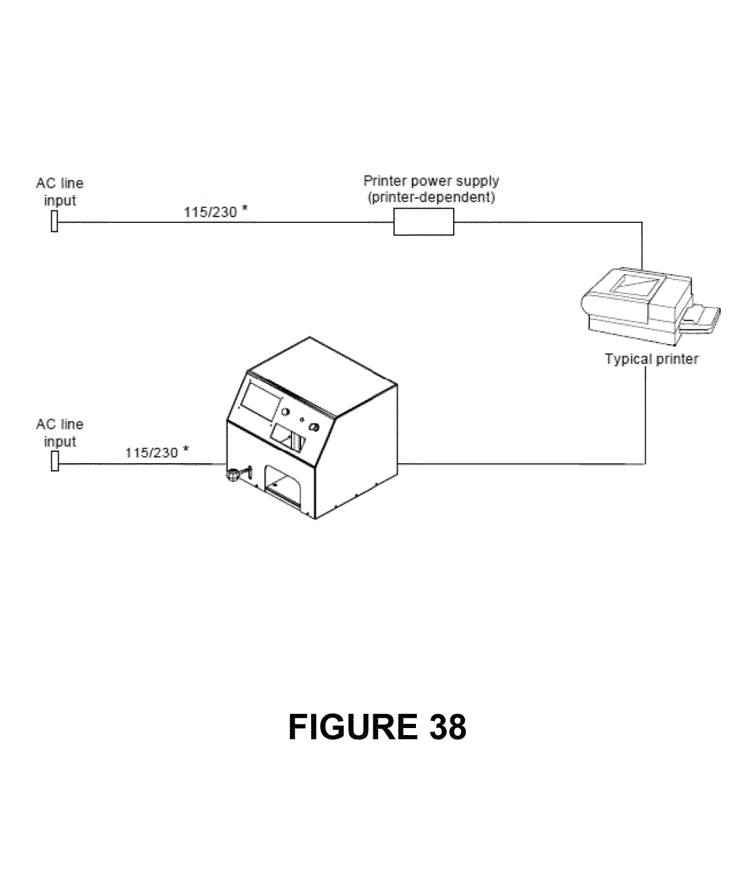

A printer may be installed in order to function with the densitometer as described below. FIG. 38 is a block diagram of an example configuration of the densitometer coupled to a printer.

In an example embodiment, information may be entered into the densitometer via a touch screen. An operator may enter information and may initiate a BMD test by using the touch-sensitive LCD screen. The touch screen may react to the contact of the operator's finger.

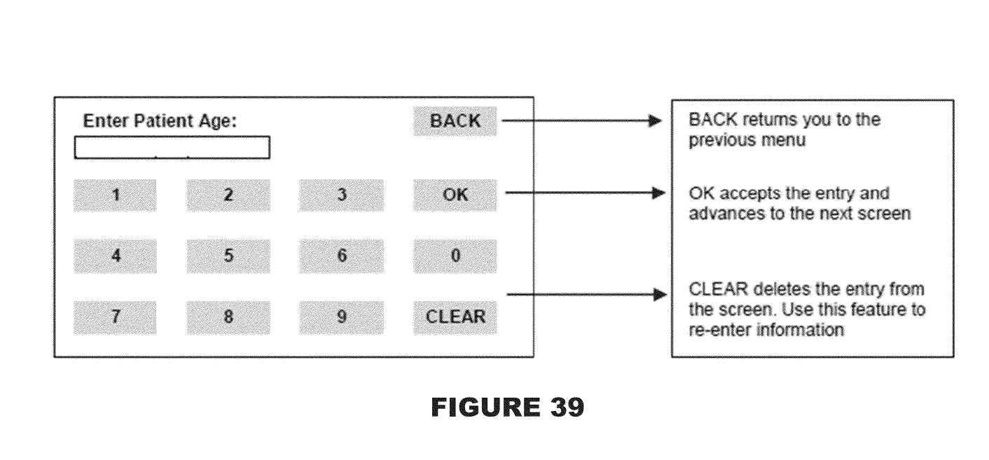

FIG. 39 is an example illustration of some of the on-screen features based on age of the AccuDEXA® densitometer appear below using the Age.

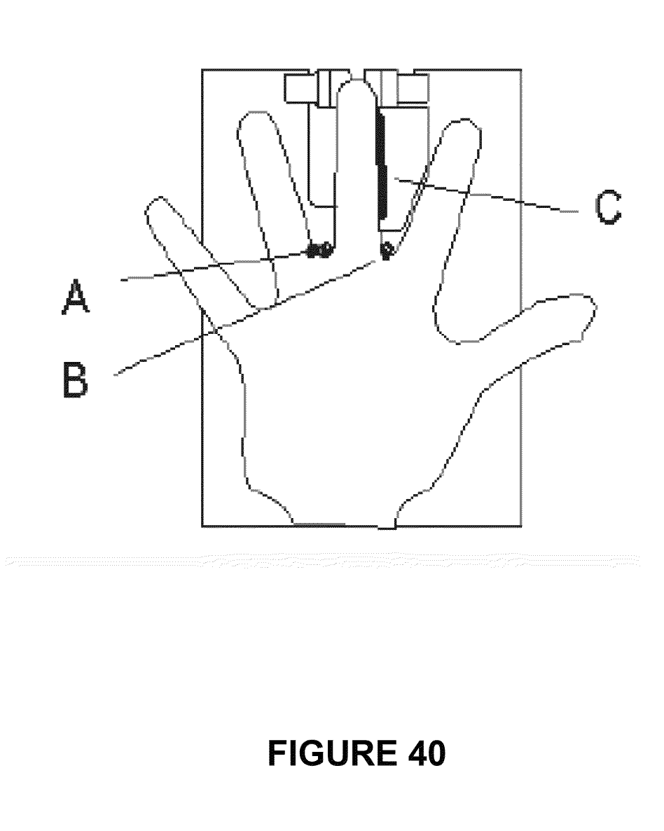

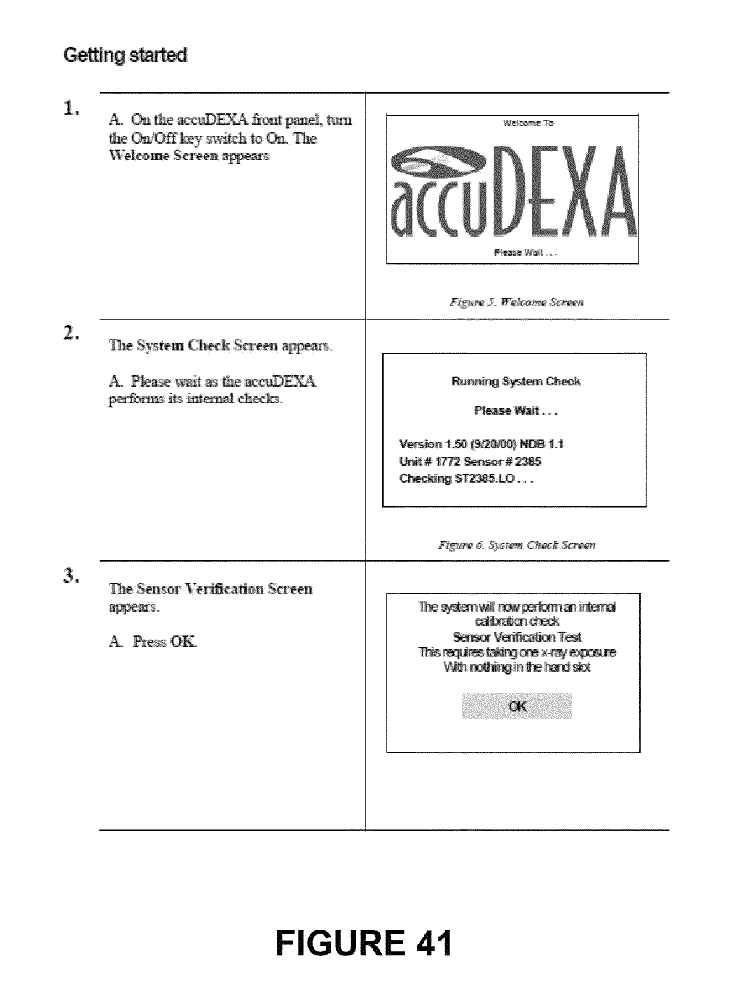

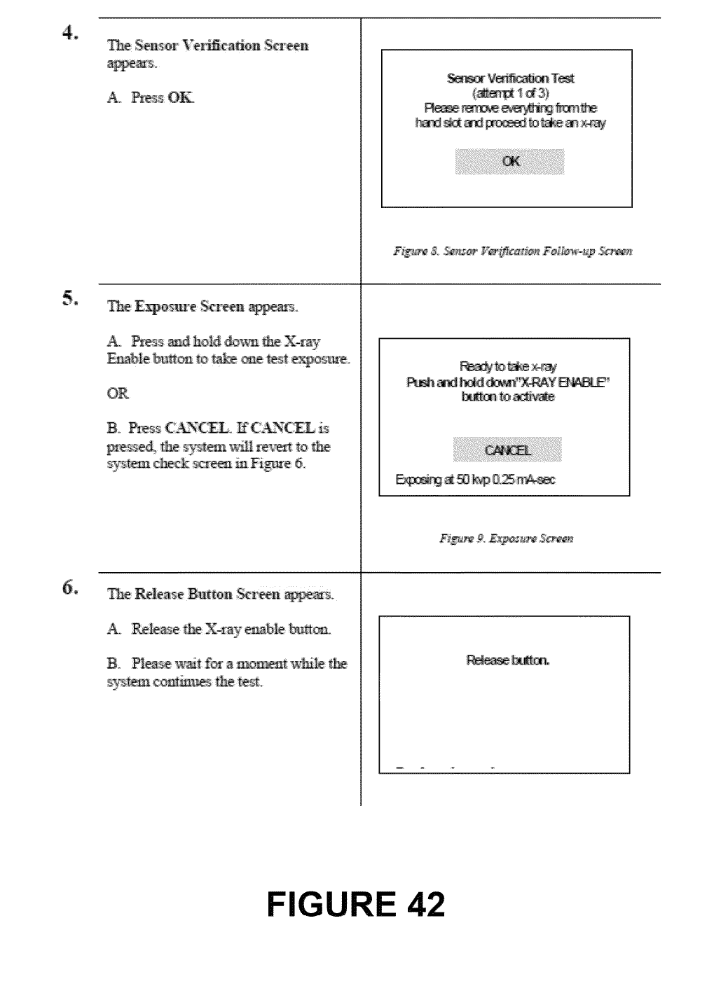

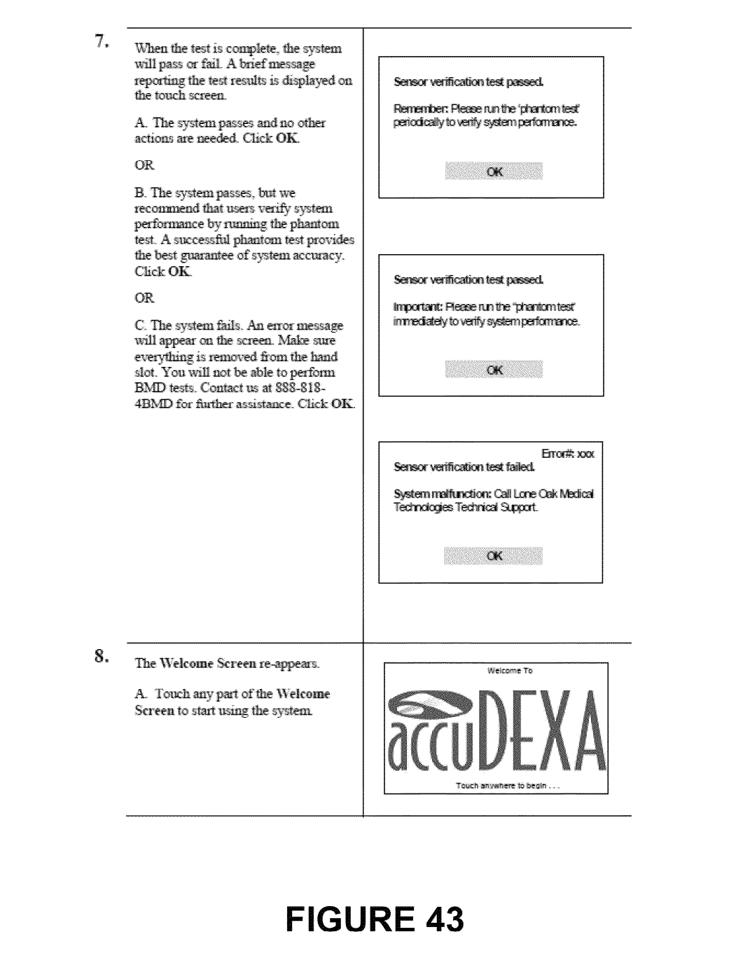

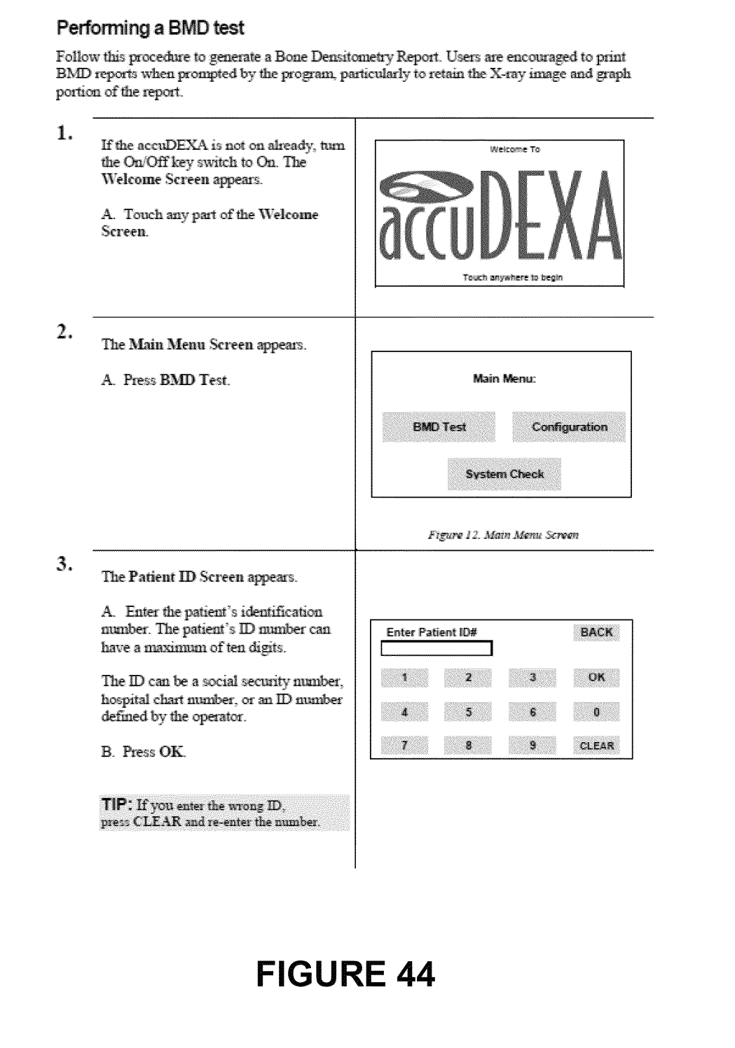

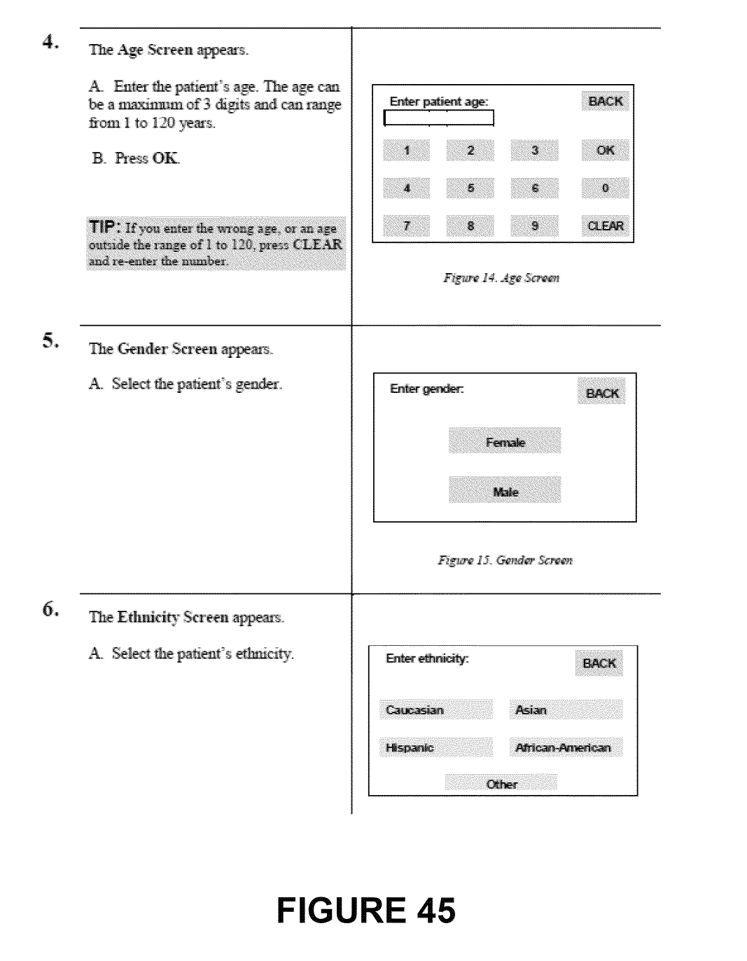

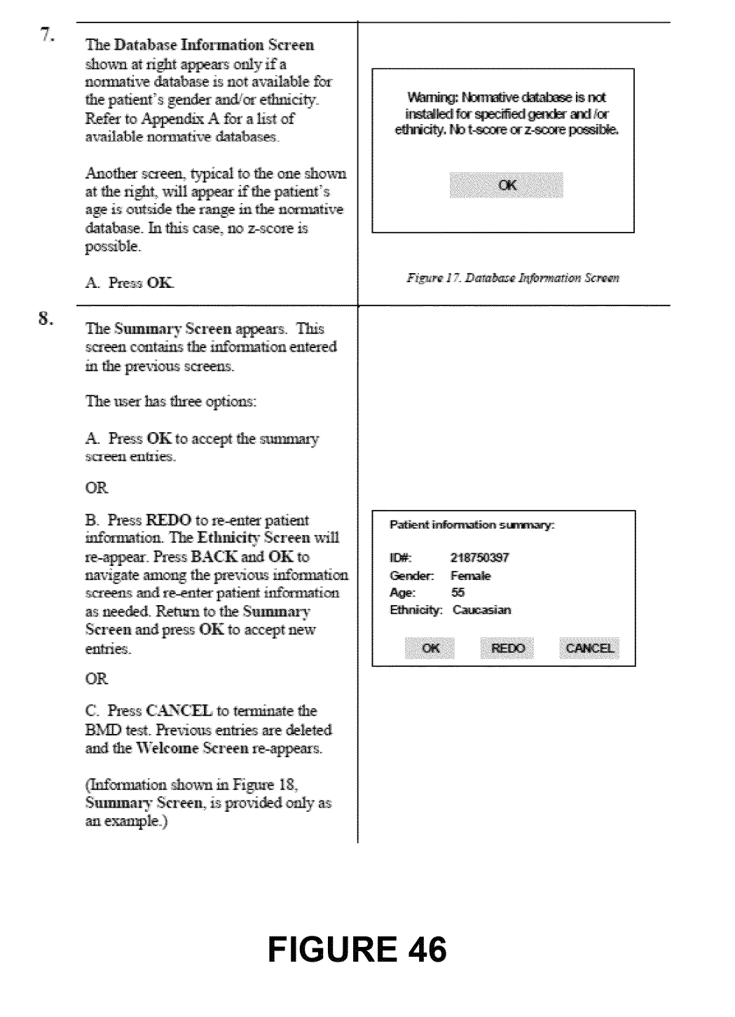

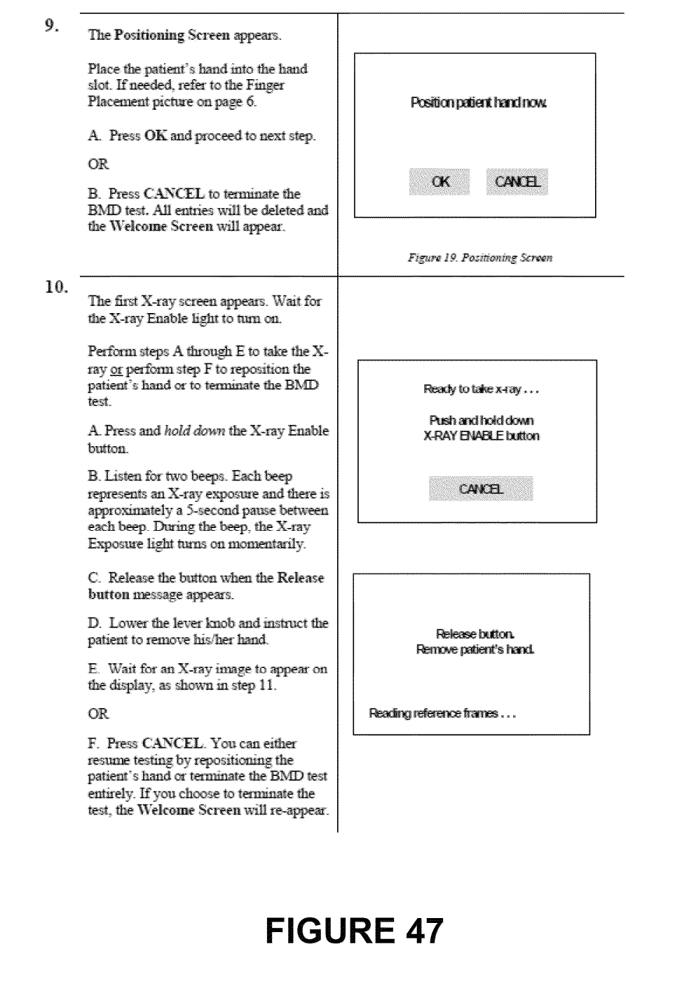

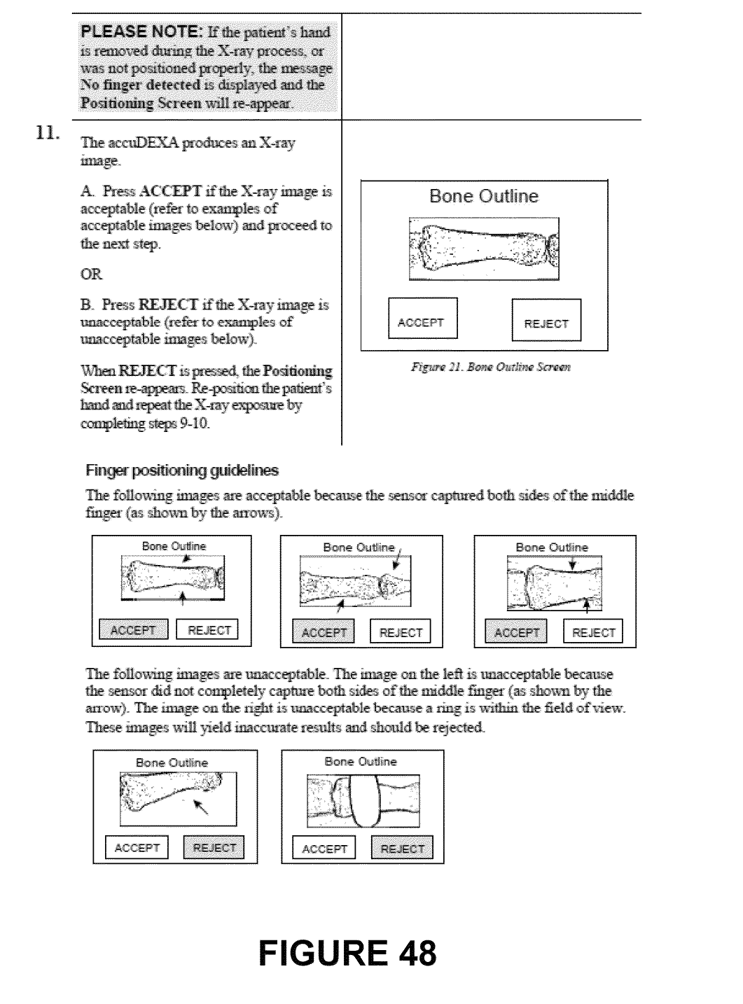

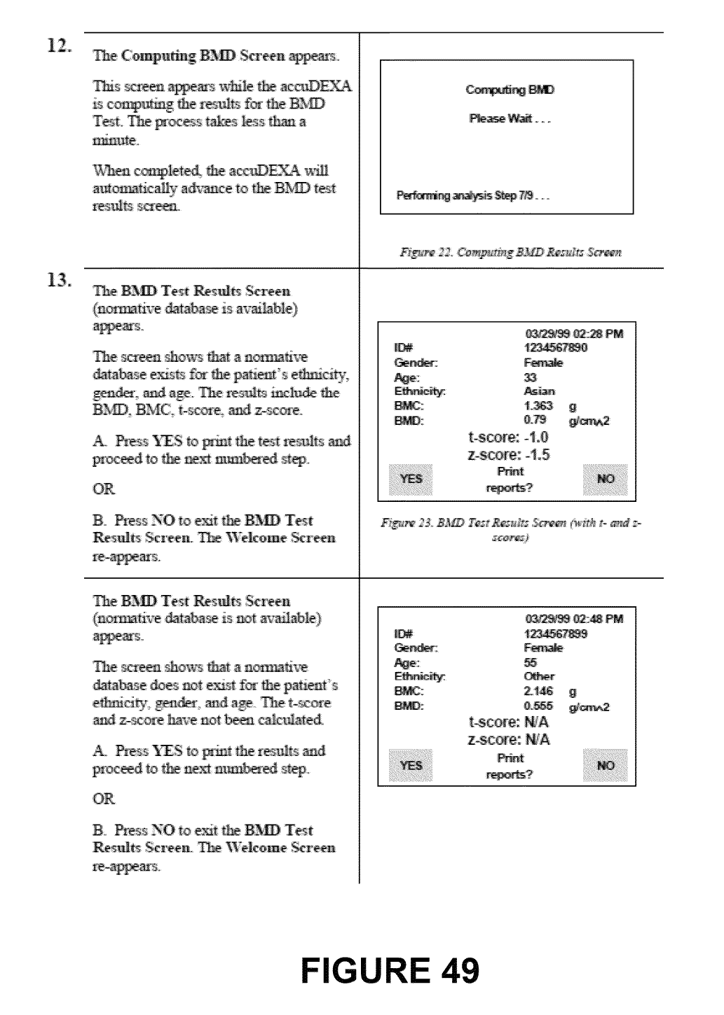

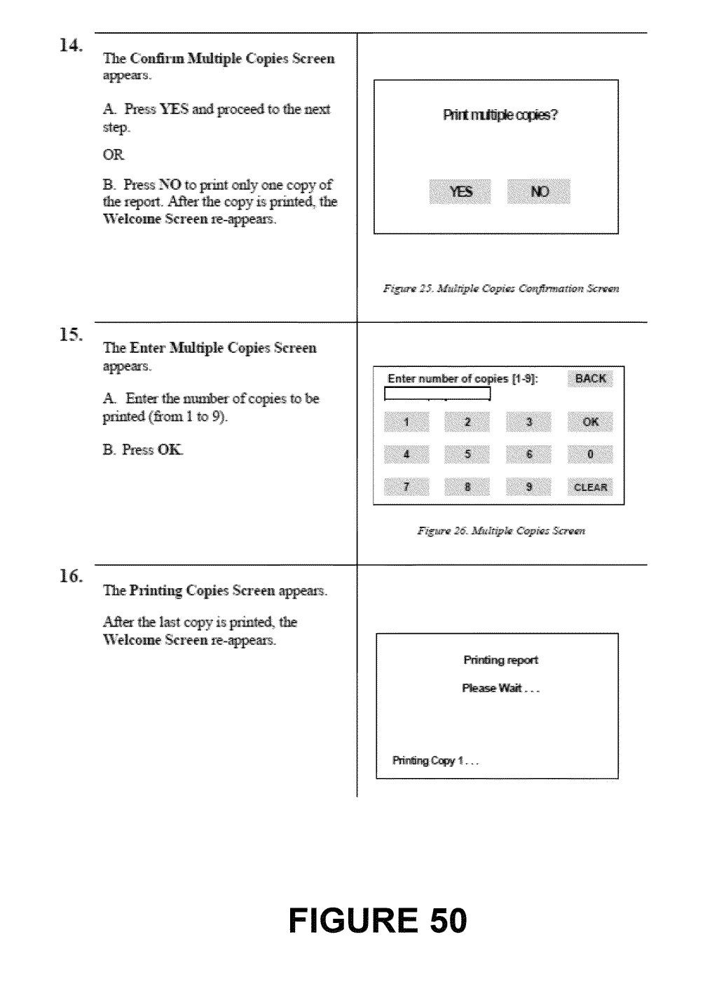

To appropriately position a finger in the AccuDEXA® densitometer, a handle knob may be pushed down. This will raise two levers located in the hand slot. The patient may be instructed to place his/her non-dominant hand inside the hand slot. For example, if the patient is right handed, the patient should place his/her left hand into the hand slot. If the patient is left handed, the patient should place his/her right hand into the hand slot. In an example embodiment, the patient's hand may be placed palm down and rest as far forward as possible, as depicted in FIG. 40. As illustrated in FIG. 40, a hand may be positioned to contact pegs at both sides of the middle finger at points A and B. The middle finger may rest firmly against the guard at C. The handle knob may be slowly released. This will lower two levers onto the patient's middle finger (one lever will rest near the tip of the finger and the other will rest near the base). These levers will gently secure the finger in place during the BMD test. To ensure proper finger placement/positioning, and to ensure accurate and precise BMD test results, all hand and wrist jewelry should be removed. Removing jewelry may improve finger positioning, increase patient comfort and help the patient to remain still during the procedure. Incorrect positioning or finger movement during testing may lead to inaccurate test results.

If jewelry cannot be removed, extra care should be taken to ensure correct positioning. For example, a ring may prevent a patient from resting his/her finger against the finger guide. As long as the finger placement approximates the description provided herein, and the X-ray image contains no part of a ring or jewelry, the exam may be valid.

In order to obtain successful BMD test results, the operator may follow several simple guidelines. The patient's hand may be positioned palm down and held motionless throughout the exam. During an exam, the AccuDEXA® densitometer may rest on a table roughly 30 inches from the floor. Patients may be in a comfortable position during the BMD Test. The patient's seat may be stationary and approximately 18 inches from the floor. The AccuDEXA® densitometer may be operated within predetermined temperature and humidity ranges.

In an example embodiment, the operator may ensure that an audible signal is heard for each of the two X-ray exposures that occur during the BMD test, the radiation label is affixed and visible on the front panel of the densitometer and a small indicator (X-ray Exposure Light) is illuminated during each exposure, and the AccuDEXA® densitometer performs a system check each time the device is powered on. The software may also perform an internal calibration before the X-ray exposures are taken and before the BMD values are calculated. If the system check or the internal calibration is unsuccessful, an error message may be displayed on the LCD screen. If the problem cannot be corrected, the error message number may be noted. And assistance may be obtained by referencing the error message number.

Note, during BMD tests, the AccuDEXA® densitometer may verify X-ray exposures as they are taken. This verification calculates the difference between high and low energy exposure to ensure that only X-rays taken at the correct energy and exposure times are accepted.

FIG. 41 through FIG. 50 depict an example process for using the AccuDEXA® densitometer.

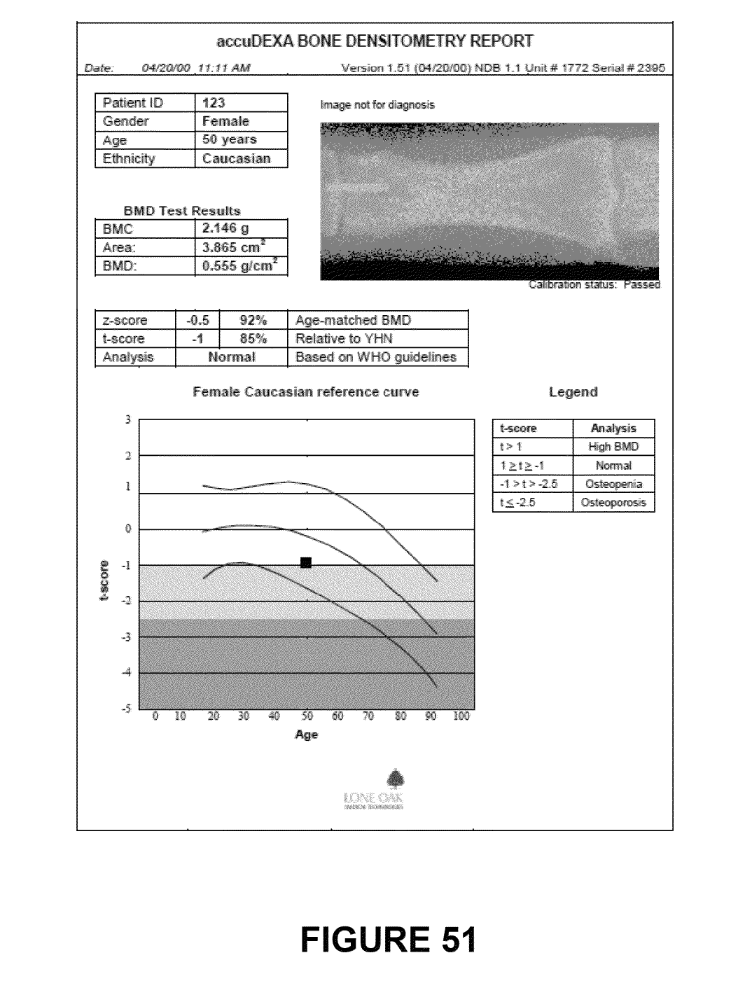

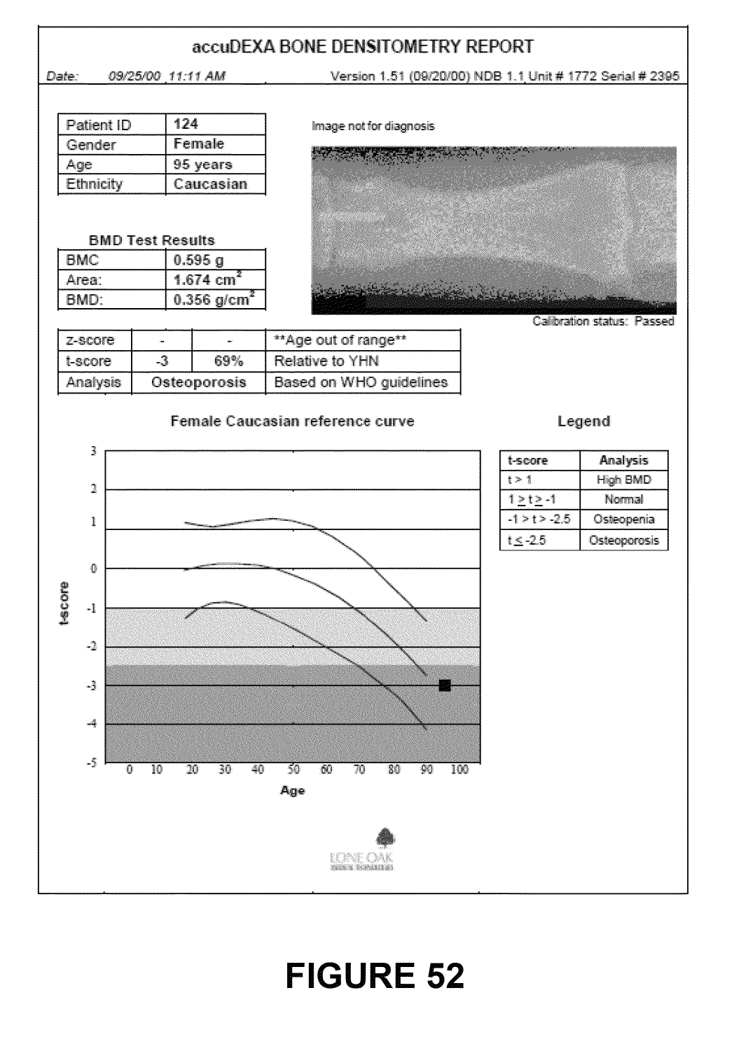

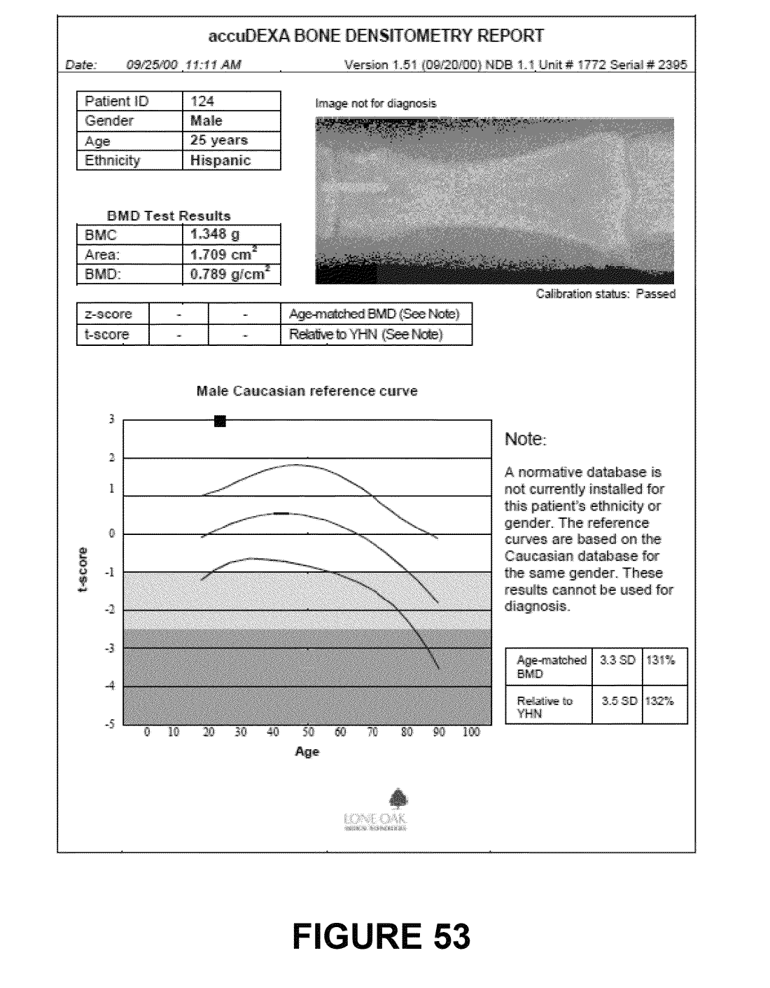

FIG. 51, FIG. 52, and FIG. 53 show examples of bone densitometry reports. The reports in FIG. 51, FIG. 52, and FIG. 53 share some common features, including general report information (report date and time, software version, and device serial number), patient information (Patient ID, Gender, Age, and Ethnicity), and BMD test information (X-ray image area and BMC and BMD results). There also are some report differences as described below.

In FIG. 51 a patient's BMD results were compared with an available normative database. The t-score was calculated from the BMD results of the patient and a database population matching the patient's gender and ethnicity. The z-score was generated using those same parameters (gender and ethnicity) and the patient's age.

In FIG. 52 a patient's BMD results also were compared with an available normative database. In this report, however, the z-score was not calculated because the patient's age (95) was “out of range” and could not be matched with an equivalent age in the database.

In FIG. 53 a patient's BMD results were generated but were not compared to a database that matched the patient's ethnicity and gender. Instead, the report graphs the results using reference curves based on the Caucasian database for the same gender.

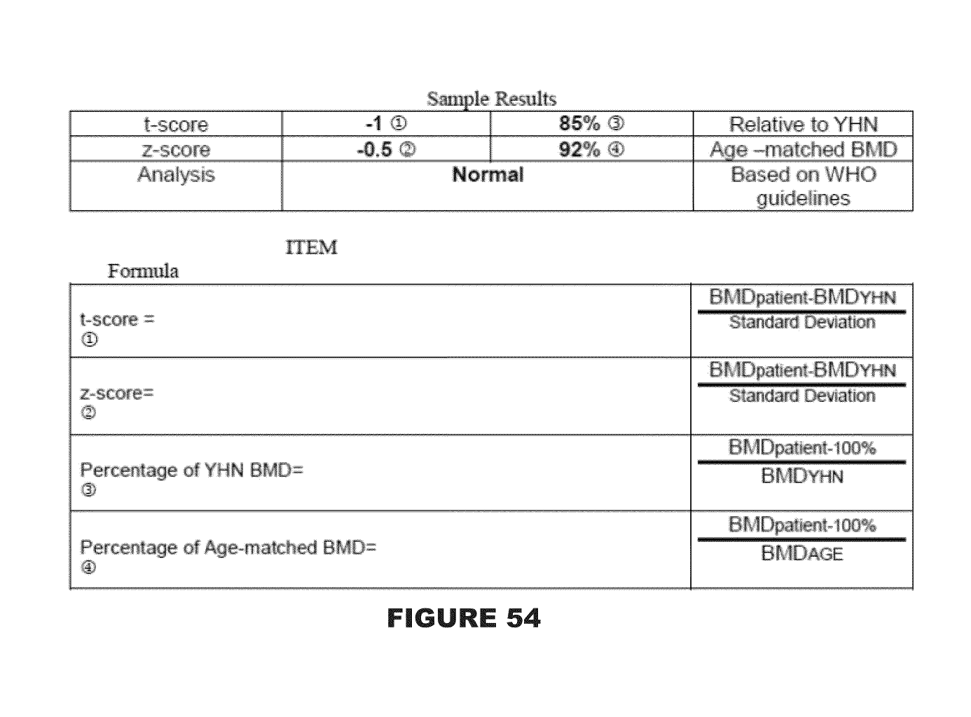

The formulas depicted in FIG. 54 may be used by the AccuDEXA® densitometer to calculate t-scores, z-scores, and to provide, as a percentage, where those scores lie in relation to the mean BMD. The analysis may be calculated automatically, based on t-score, and reported as Normal, Osteopenia, or Osteoporosis.

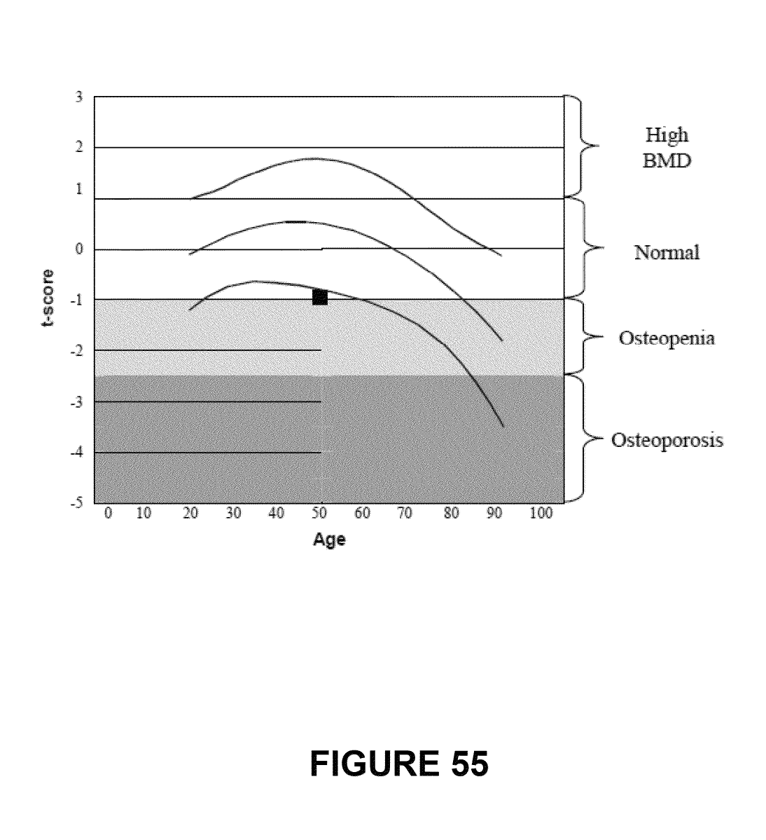

FIG. 55 depicts sample graphs of t-scores versus age. On the sample reference curve shown in FIG. 55, the scale of t-scores is shown at the left and the scale for age is at the bottom. The three curved lines are isometric z-scores. The top curve represents one standard deviation above the age-matched mean BMD. The middle curve represents the age-matched mean BMD. The bottom curve represents one standard deviation below the age-matched mean BMD. Isometric t-scores are displayed on the y-axis. The t-scores can be positive or negative and correspond to standard deviation increases or decreases in BMD as compared to a young, healthy normal (YHN) individual. The range of ages for z-scores is displayed on the x-axis. The t-score and z-score for the scanned patient can be seen graphically on the curve, and is represented by a small square box. In this example the patient has a lower than mean BMD compared to a young healthy normal (t-score) and age-matched (z-score) database.

Bone mineral estimates may be used to provide an index of fracture risk. Individuals who fall below the range of young healthy normal individuals may be at a greater risk for fracture. The World Health Organization (WHO) has established four general diagnostic categories that define categories for low bone density as shown in the table below.

| | |
|---|---|
| Normal | A value for bone mineral density (BMD) or bone |
| | mineral |
| | content (BMC) within 1 standard deviation (SD) |
| Low Bone Mass | A value for BMD or BMC more than 1 SD below the |
| (osteopenia) | young |
| Osteoporosis | A value for BMD or BMC of 2.5 SD or more below |
| | the young adult mean. |
| Severe | A value for BMD or BMC more than 2.5 SD below |
| Osteoporosis | the young adult mean in the presence of one or more |
| | fragility fractures. |

The AccuDEXA® densitometer may automatically calculate a patient's risk based on the t-score and may report the results as Normal, Osteopenia, or Osteoporosis.

While low BMD may be a factor in determining a patient's risk for fracture, there may be other factors that also contribute to risk. Patients with a combination of several risk factors are at an increased risk of fracture. The following is a summary of risk factors.
Being female
A small, thin frame
Advanced age
A family history of osteoporosis
Early menopause
Abnormal absence of menstrual periods (amenorrhea)
Anorexia nervosa or bulimia
A diet low in calcium
Use of certain medications (steroids, anticonvulsants, excessive thyroid hormones,
certain cancer treatments)
Low testosterone levels in men
A sedentary lifestyle
Cigarette smoking
Excessive alcohol intake
Malabsorption problems

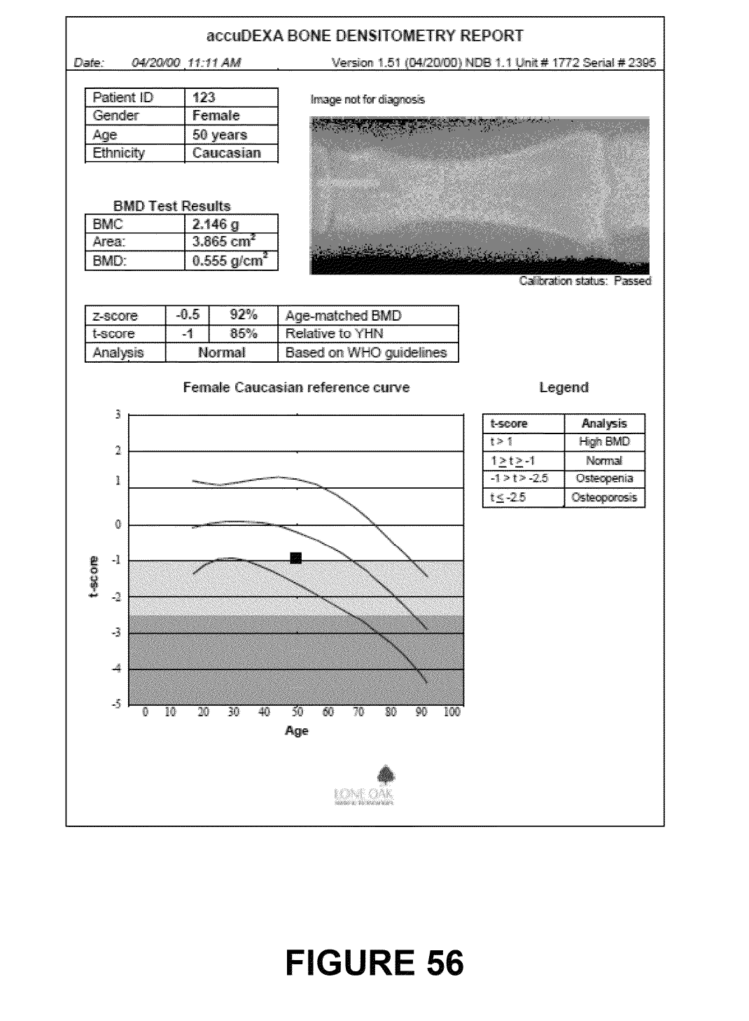

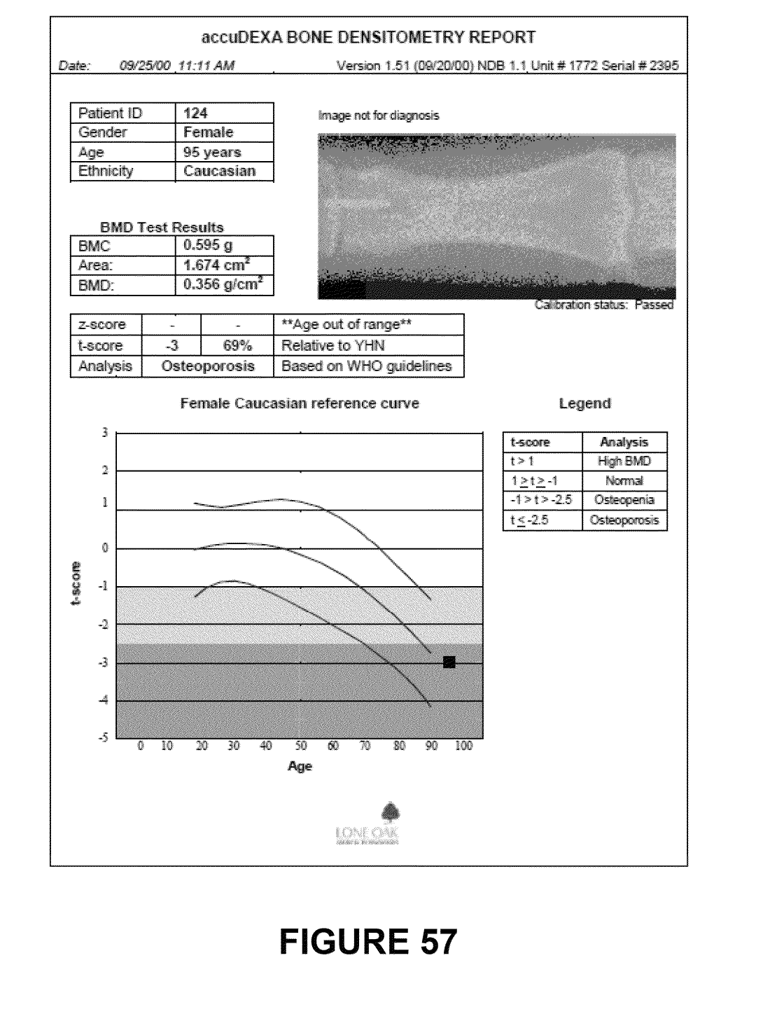

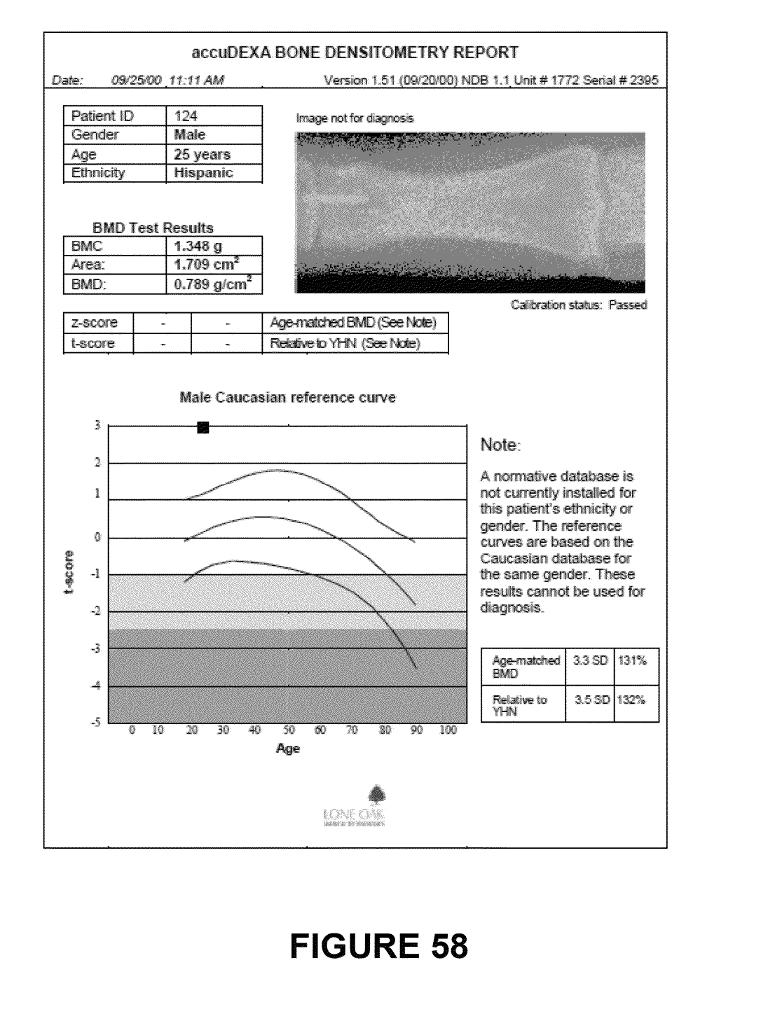

FIG. 56, FIG. 57, and FIG. 58 illustrate example densitometry reports.

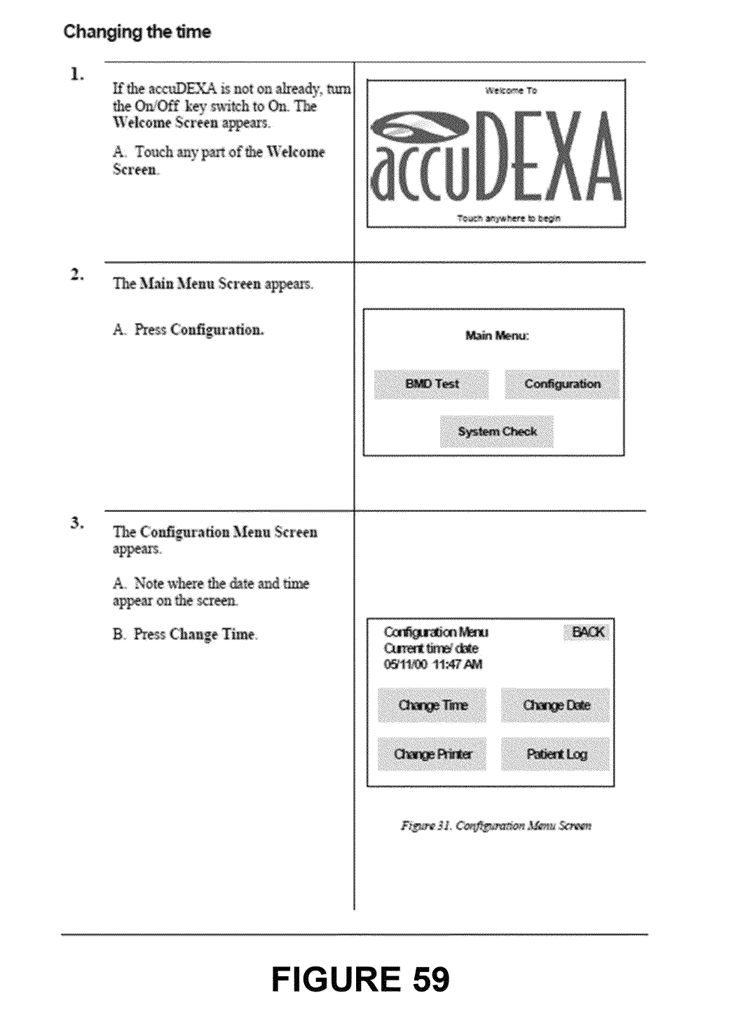

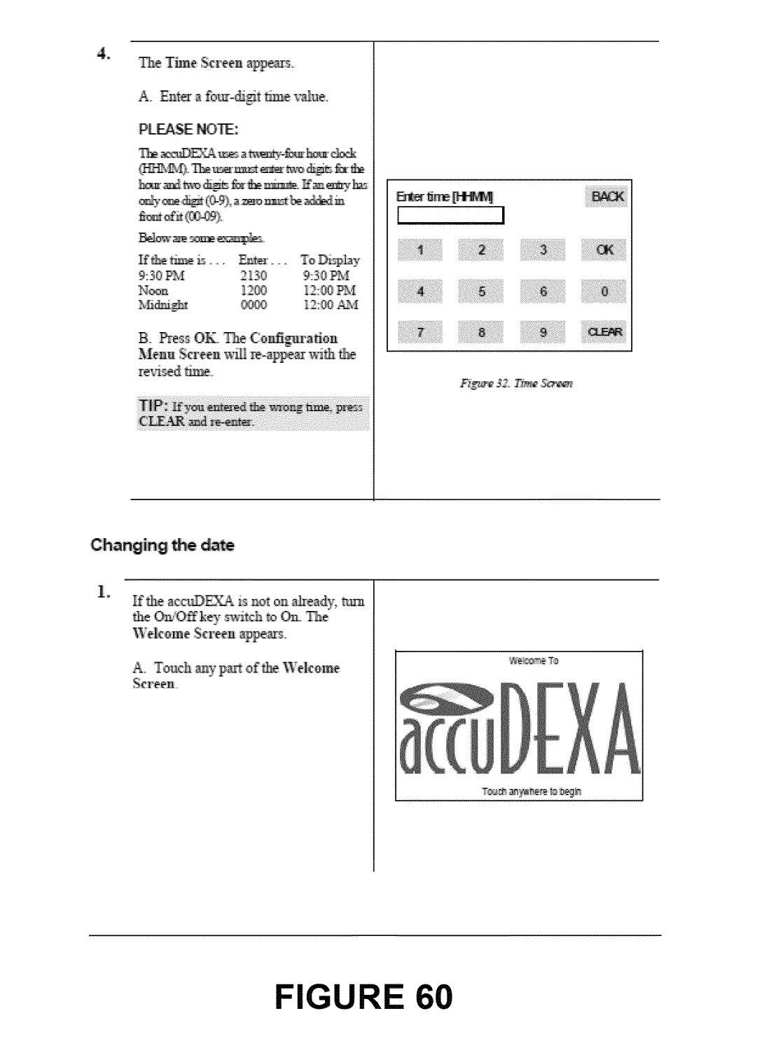

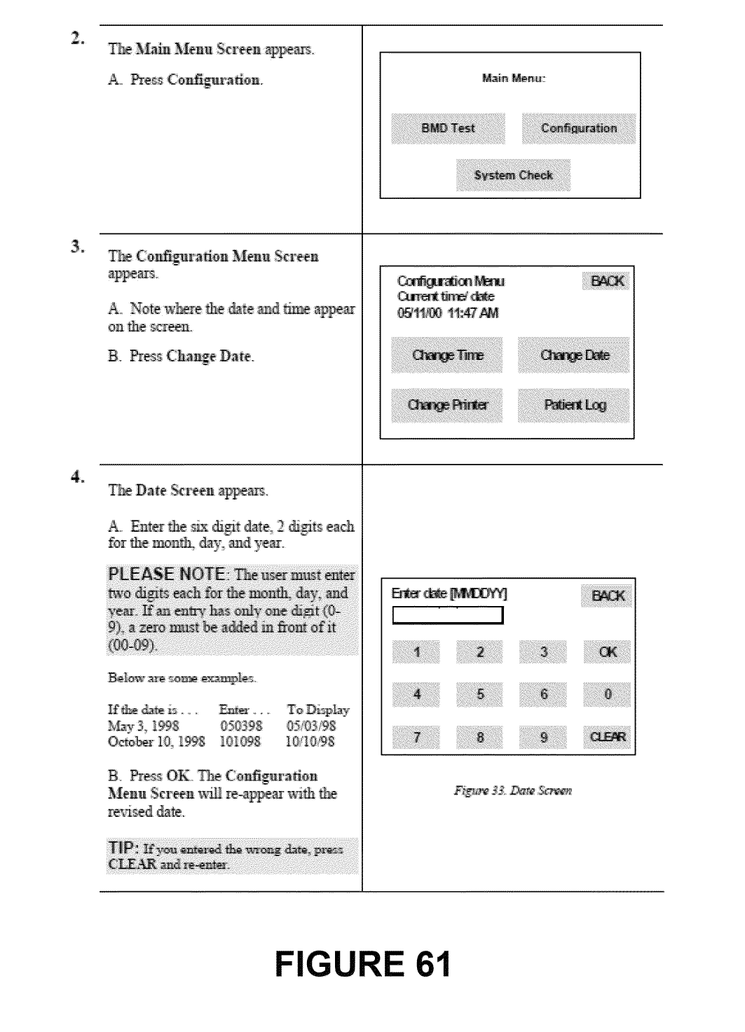

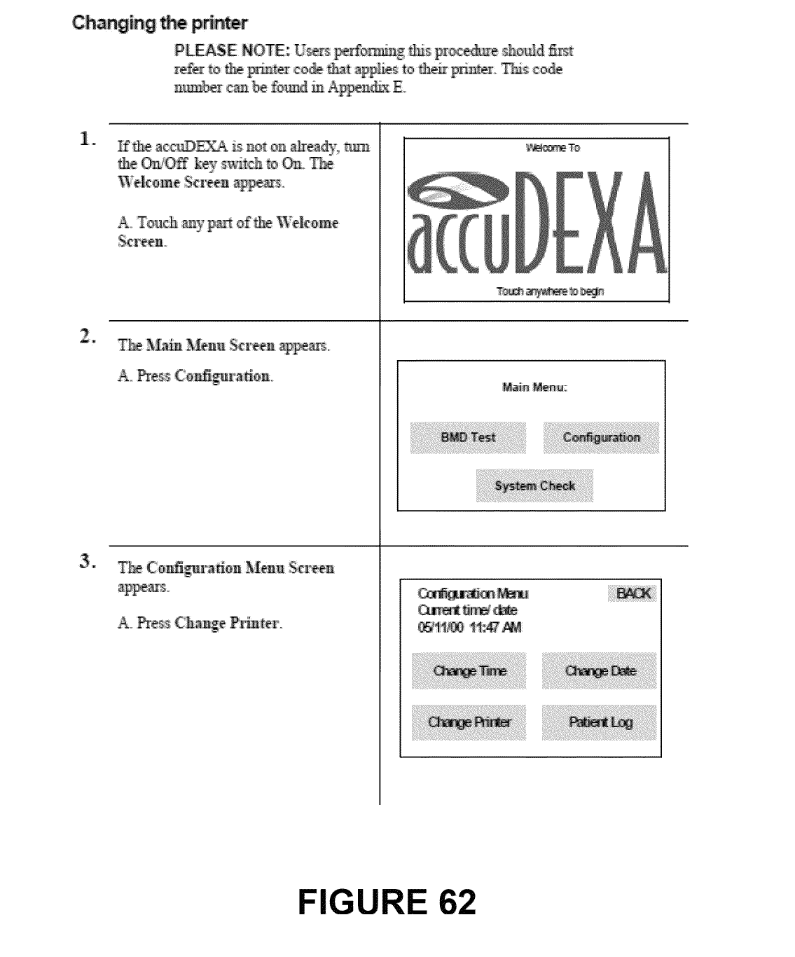

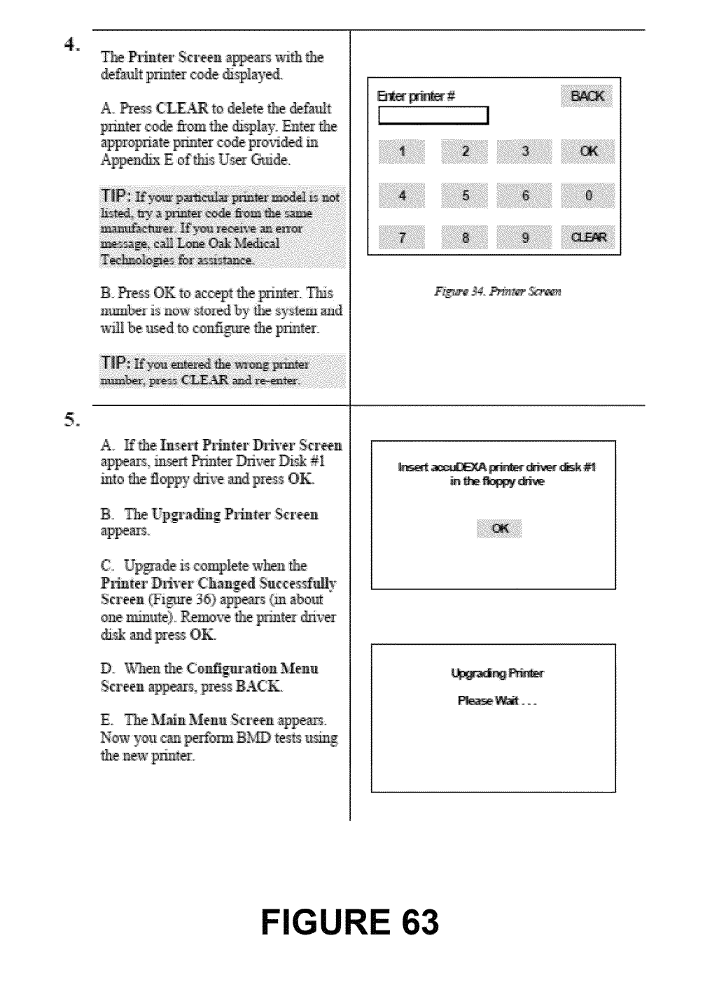

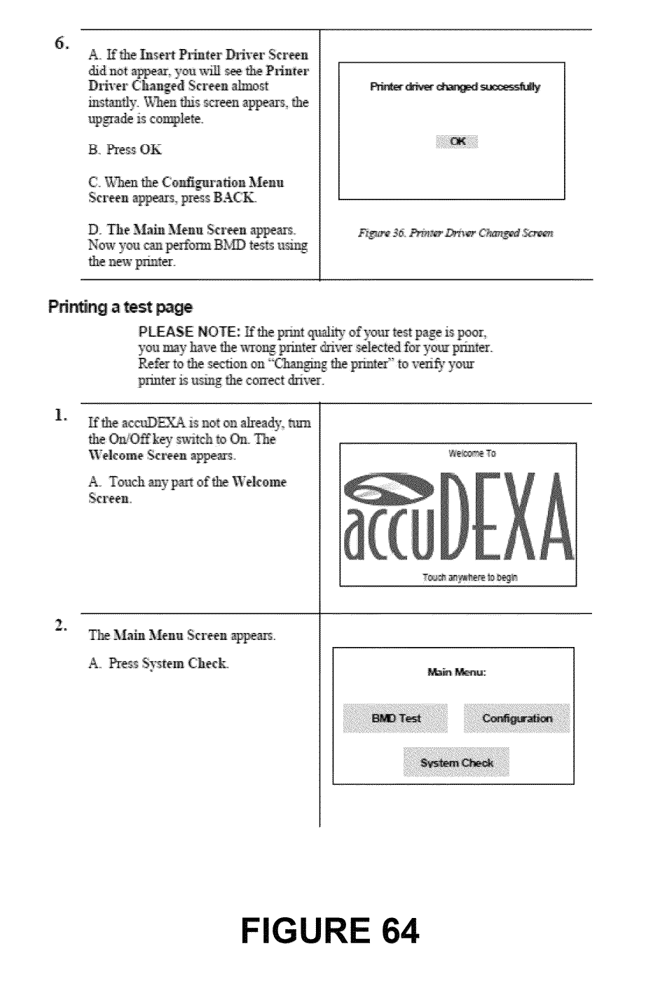

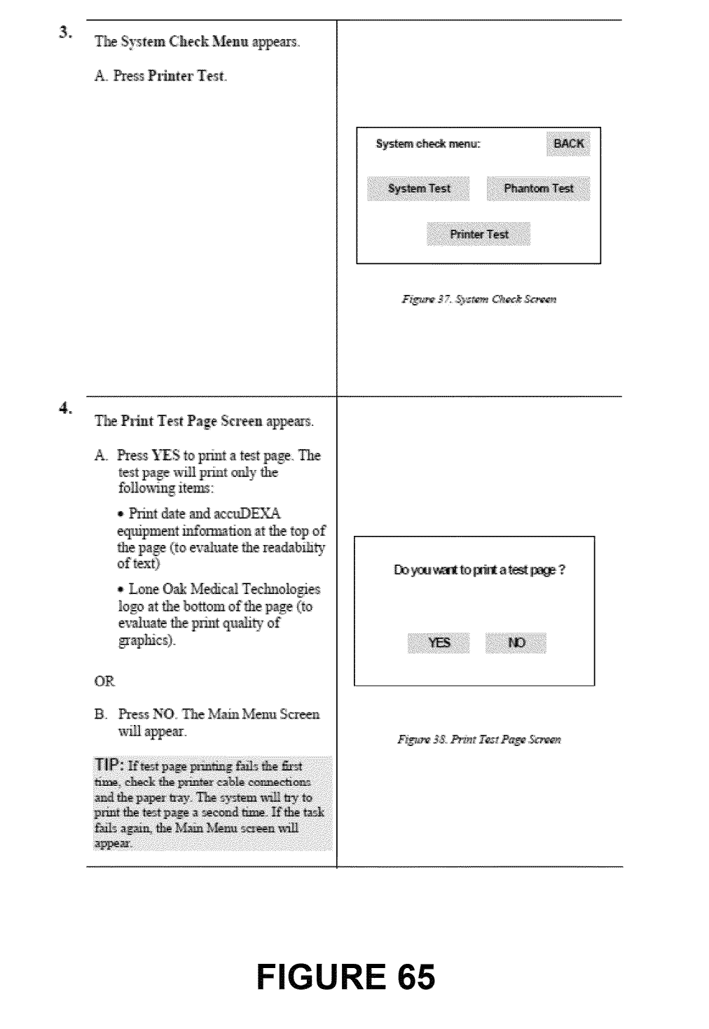

FIG. 59 through FIG. 64 depict an example process for using the AccuDEXA® densitometer.

Phantom tests may be performed utilizing the densitometer. A phantom test comprises a quality-control check of the AccuDEXA® densitometer system. It utilizes a finger phantom (article with known characteristics) and may take about 2 minutes to complete. The phantom test provides means for users to verify that the AccuDEXA® densitometer is maintaining its highest level of performance. Internally, both calibration and quality control may be performed each time the unit is turned on. More frequently, medical practitioners are being asked by insurance companies to provide quality control printouts for their diagnostic devices. Accordingly, when performing a phantom test, users may automatically be prompted to print a QC test report. Understanding phantom test results

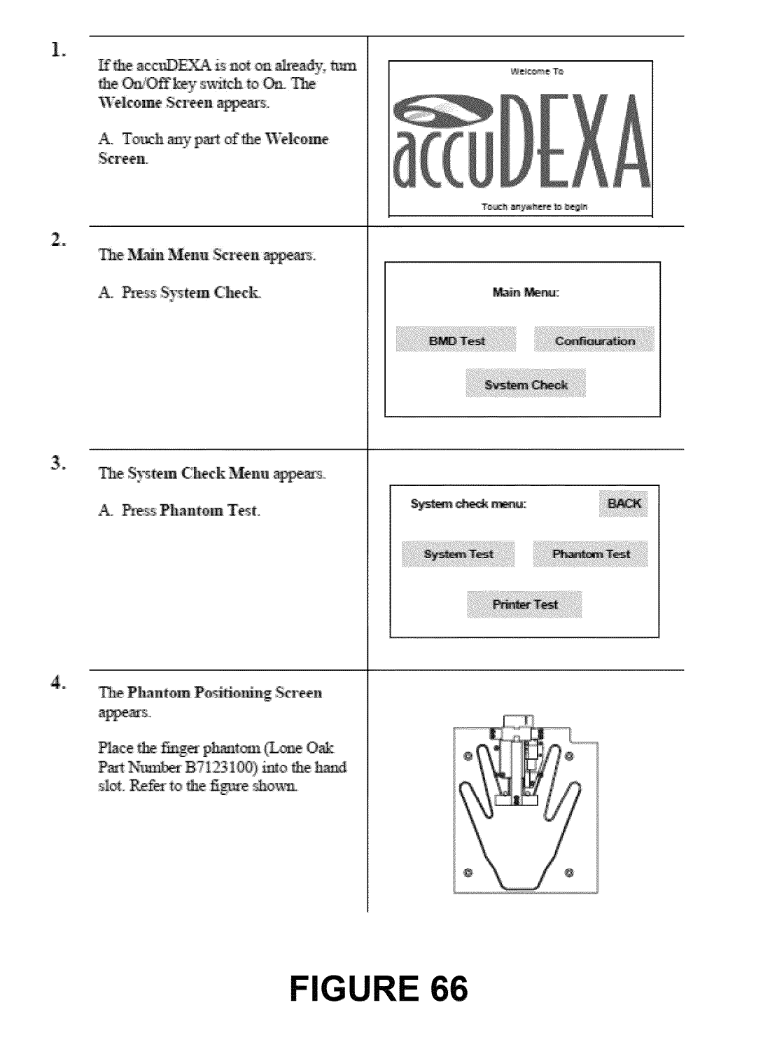

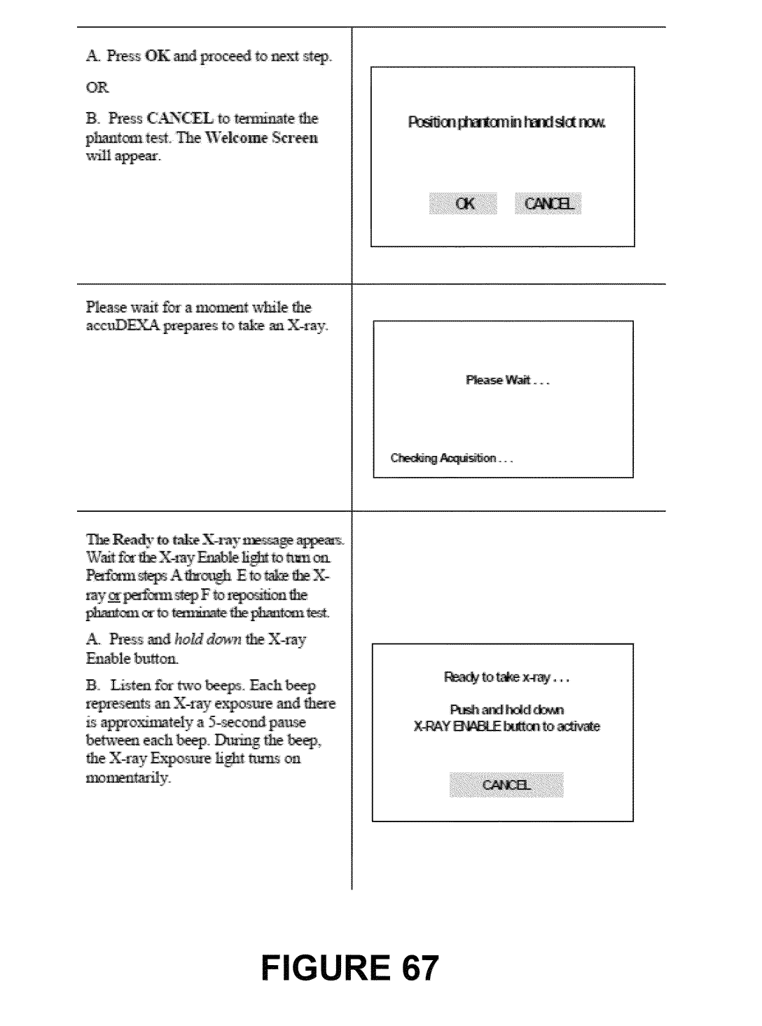

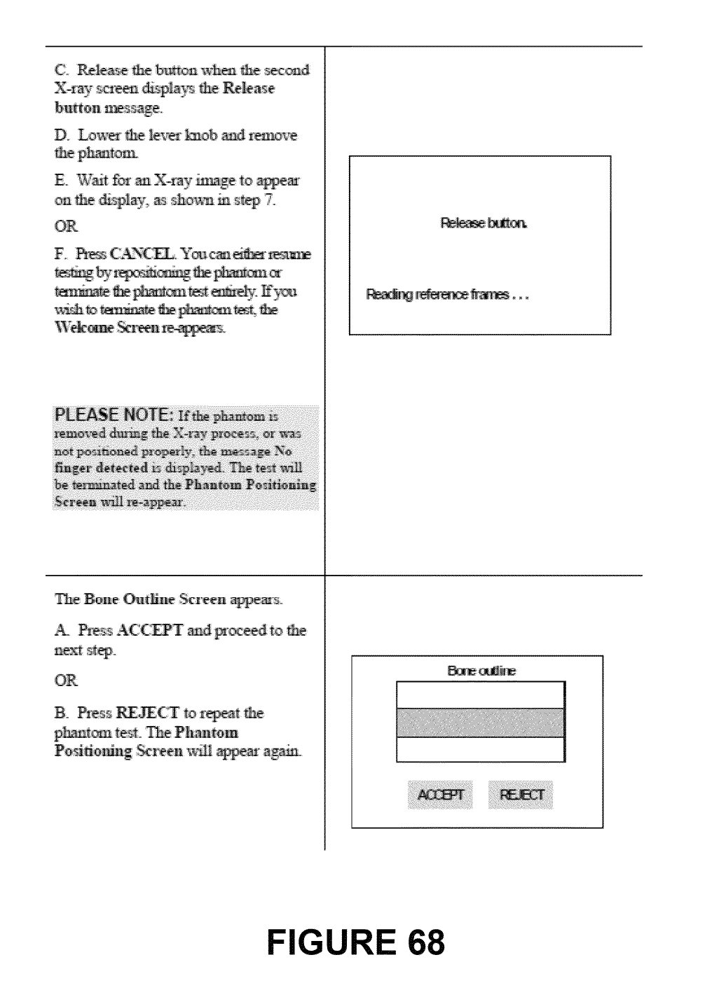

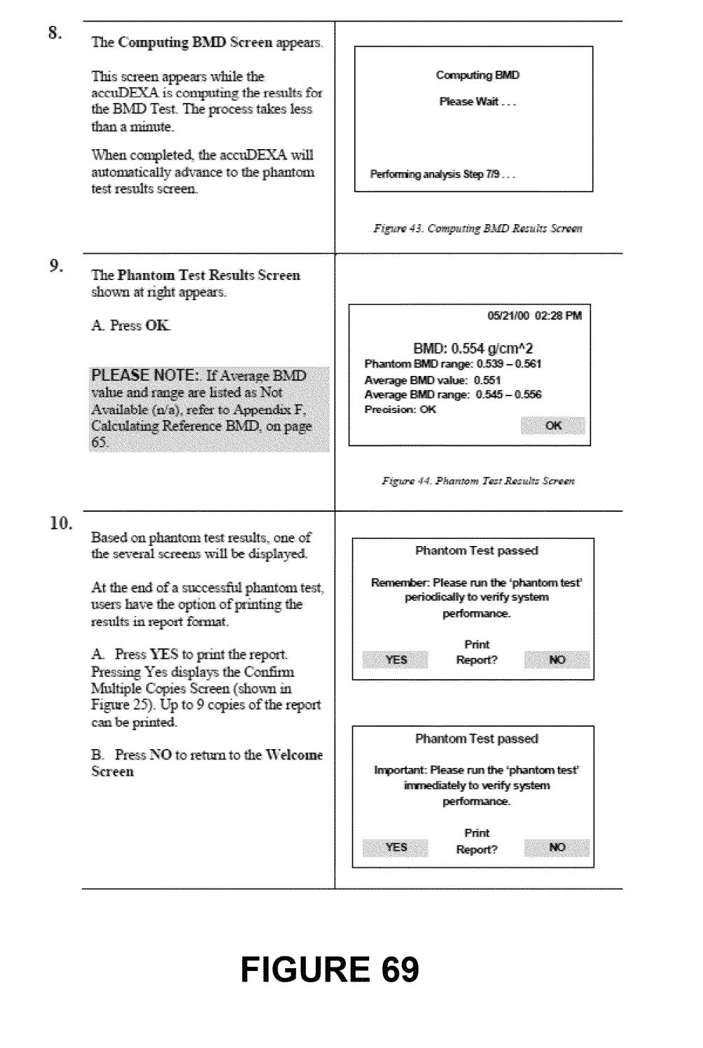

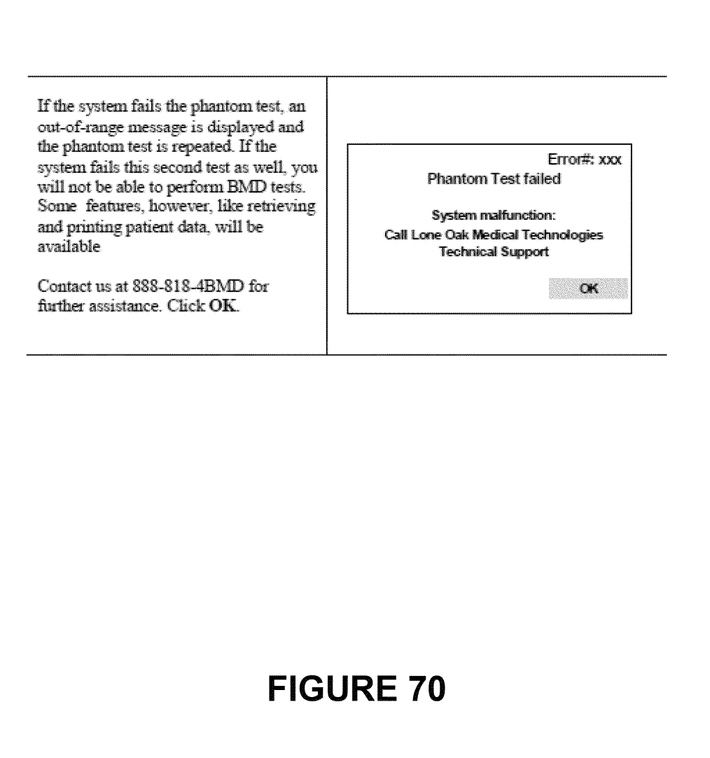

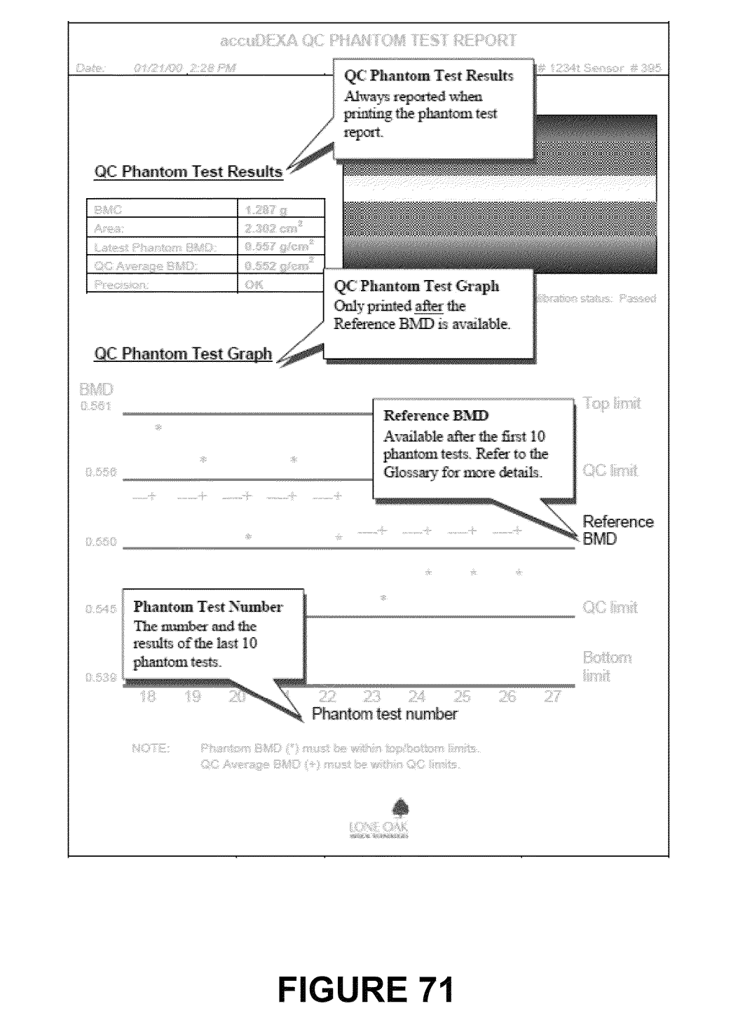

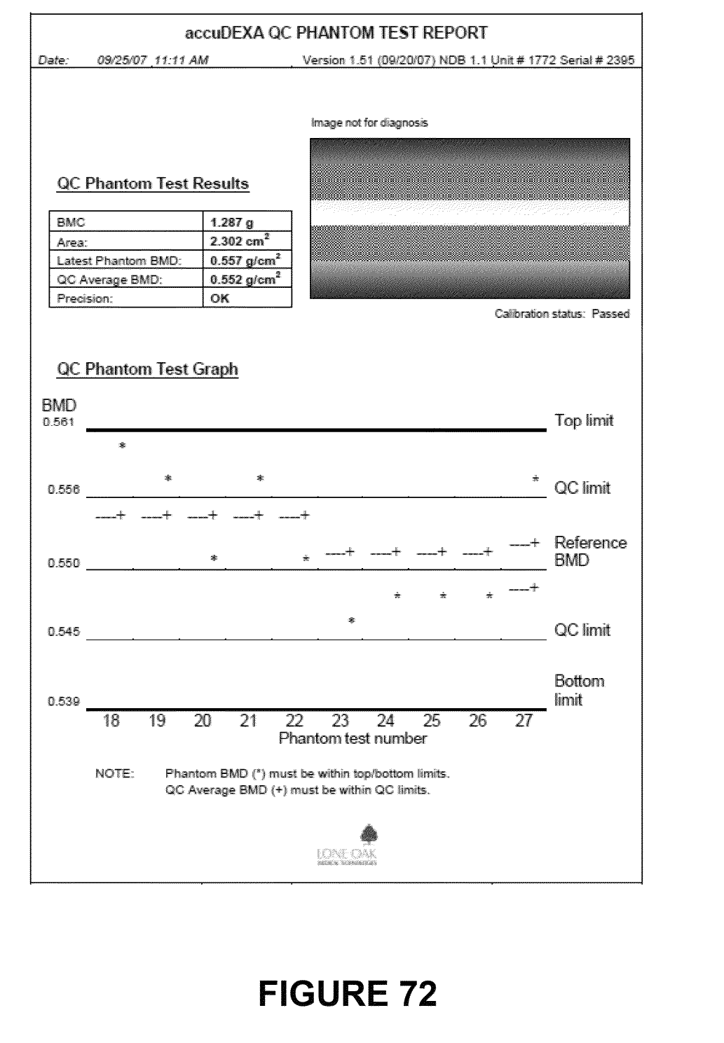

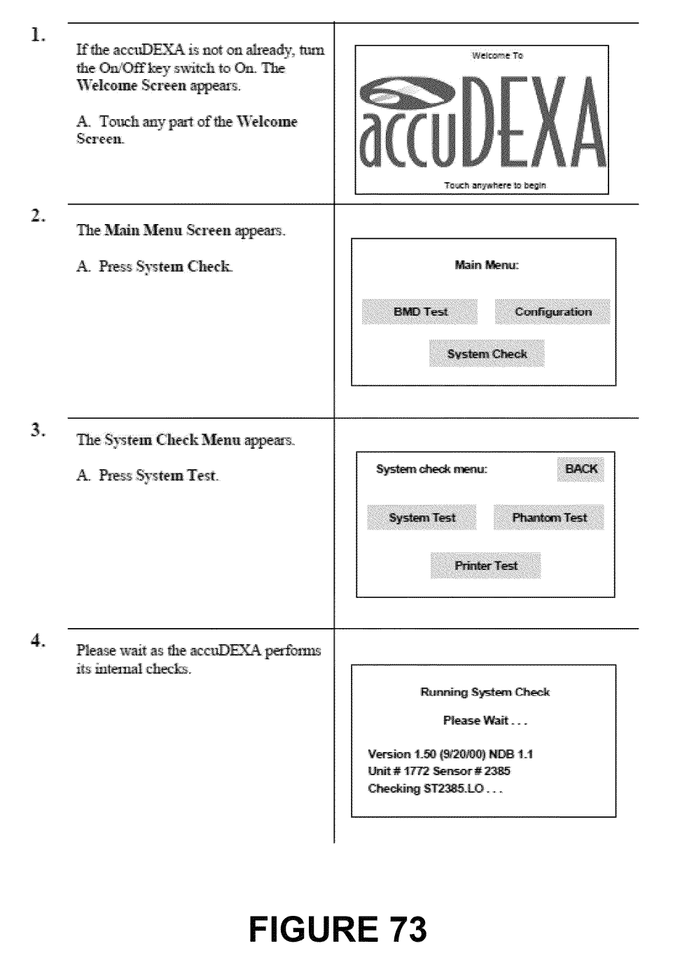

FIG. 65 through FIG. 71 illustrate an example process for performing a phantom test. FIG. 72 and FIG. 73 depict example phantom test reports. A phantom test report may comprise information about system performance. This information may be grouped in two areas: QC Phantom Test Results and QC Phantom Test Graph. Referring to FIG. 72 and FIG. 73, the QC Phantom Test Results table summarizes the results from the current phantom test and provides other information on the status of BMD testing. The result of the current phantom test is called Phantom BMD and is an indicator of how well the system compares to pre-defined limits in AccuDEXA®'s densitometer configuration file. This is one measure of system performance. A second measure of performance may comprise QC Average BMD, which considers both the current and previous Phantom Test results. QC Average BMD is a “moving average”—the result of averaging the last 10 Phantom BMD values. For this reason QC Average BMD may be an indicator of how closely the system is performing to its baseline value (Reference BMD). The QC Phantom Test Graph is plotted below the test results table. Printing the phantom report may aid in reviewing the graph. Looking at the QC Phantom Test Graph, certain trends may be observed regarding Phantom BMD and QC Average BMD results. The x-axis in the middle of the table (Reference BMD) provides the guideline for interpreting these results. When the system is performing properly, Phantom BMD values (shown as *'s on the graph) may fall within Phantom limits and QC Average BMD values (shown as +'s) may fall within QC Limits. (Limits are specified in the configuration file.). When both Phantom BMD and QC Average BMD are within the limits for the system, the precision for the unit may be considered satisfactory and is reported as OK. If precision is listed as “Out of Range”, it means that the BMD result may be outside the 0.52 and 0.58 range for acceptable results. In this event, users may be prompted for additional action.

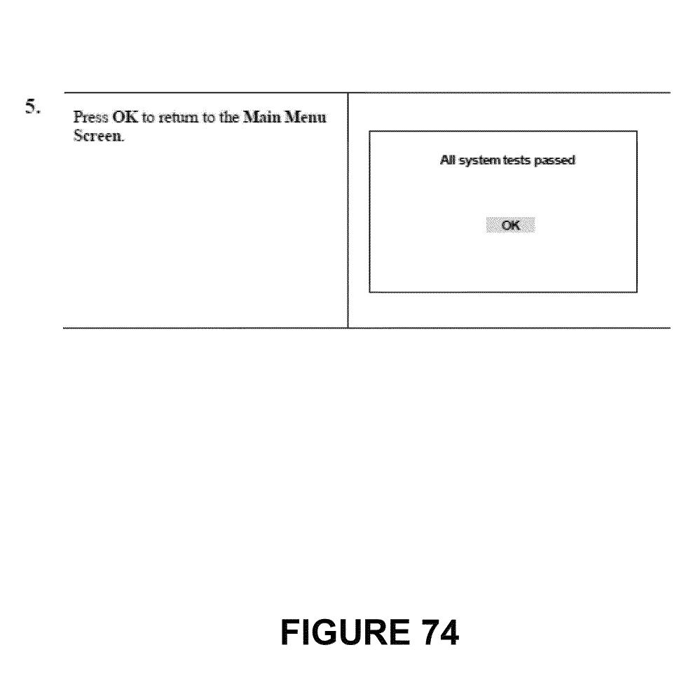

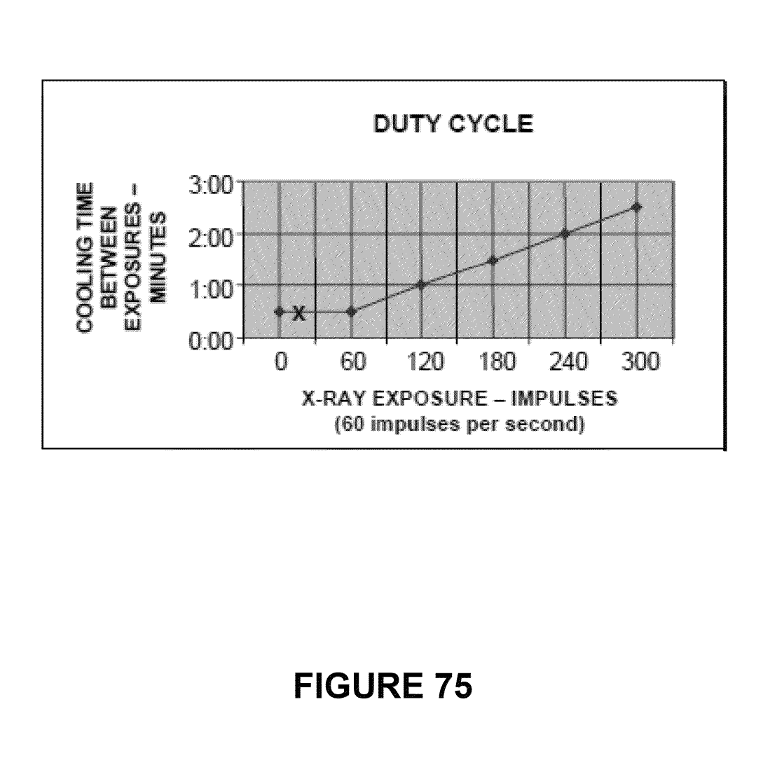

FIG. 74 and FIG. 75 depict and example process for performing a system test. A system test may initiate internal checks that may be similar to those performed automatically upon system start-up. Some checks may be performed upon system startup and not repeated during a system test.

The AccuDEXA® densitometer may perform an automatic check of its ability to operate whenever it is turned on. Components verified by this check may include software executable and system files, sensors and interfaces, and mechanical fixtures. If the device fails the system check, an error message may appear on the screen display, listing the cause of the problem. For example, if normal operating temperature limits are exceeded, the system may report, Error: System temperature too hot (70-85 F/21-29 C only) or Error: System temperature too cold (70-85 F/21-29 C only) as appropriate. A system test may be performed at any time. Other user initiated system tests that may be initiated via the system check menu may include System Test, Printer Test, and Phantom Test.

The AccuDEXA® densitometer may estimate bone mineral content (BMC, g) and bone mineral density (BMD, g/cm2) in a region of the middle phalanx of the third finger of the non-dominant hand using dual-energy X-ray absorptiometry (DEXA). The density of soft tissue may be compensated for by acquiring information at two distinct energy levels. The AccuDEXA® densitometer may emit a low-energy X-ray pulse at 50 kVp and a high-energy X-ray pulse at 70 kVp. At 70 kVp, a zinc plate may be used to filter out the low energy X-rays. An epoxy and an aluminum finger wedge of known density may be aligned in the field of view (FOV) of the sensor. The known density of the wedge may be used in bone density estimation, allowing for a relationship to be established between X-ray attenuation and density, which may be applied to every pixel of the X-ray sensor in the FOV. Furthermore, inclusion of the wedge within the FOV may allow a calibration test to be performed during each exam.

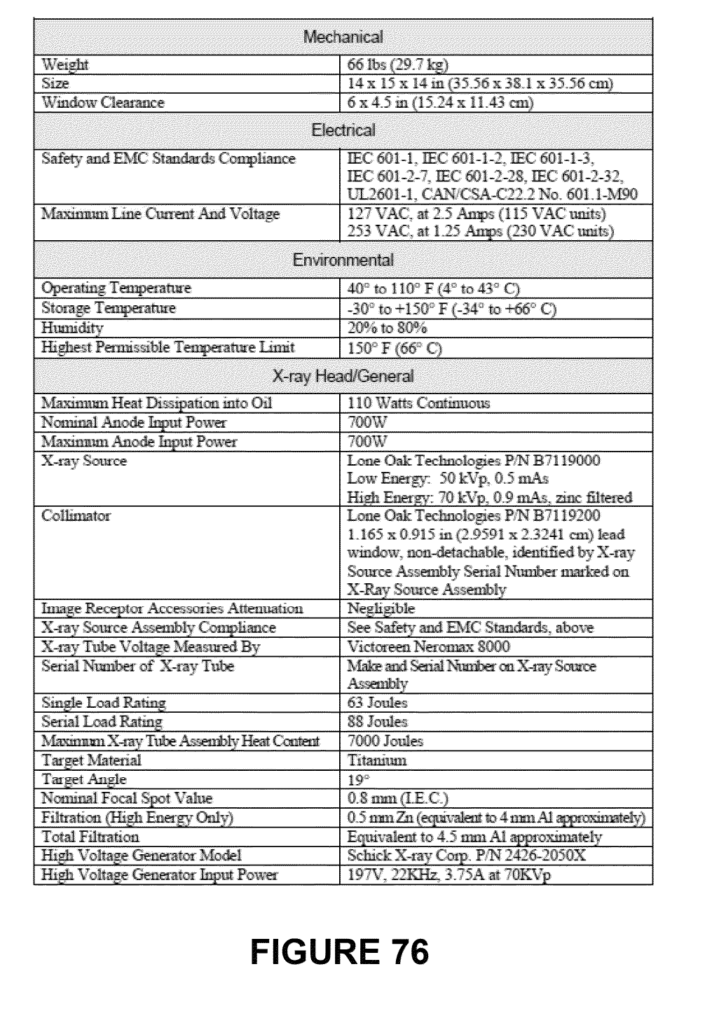

The x-ray mechanism of the densitometer may utilize a duty cycle as depicted in FIG. 76. A densitometer pulse may last approximately 0.14 seconds, which is equivalent to 8.4 pulses as indicated by the x in the FIG. 76. The densitometer may utilize two X-ray exposures as depicted in the table below.

| Impulse | Duration |
|---|---|
| High Energy | .09 seconds (maximum) |
| Low Energy | .06 seconds (maximum) |

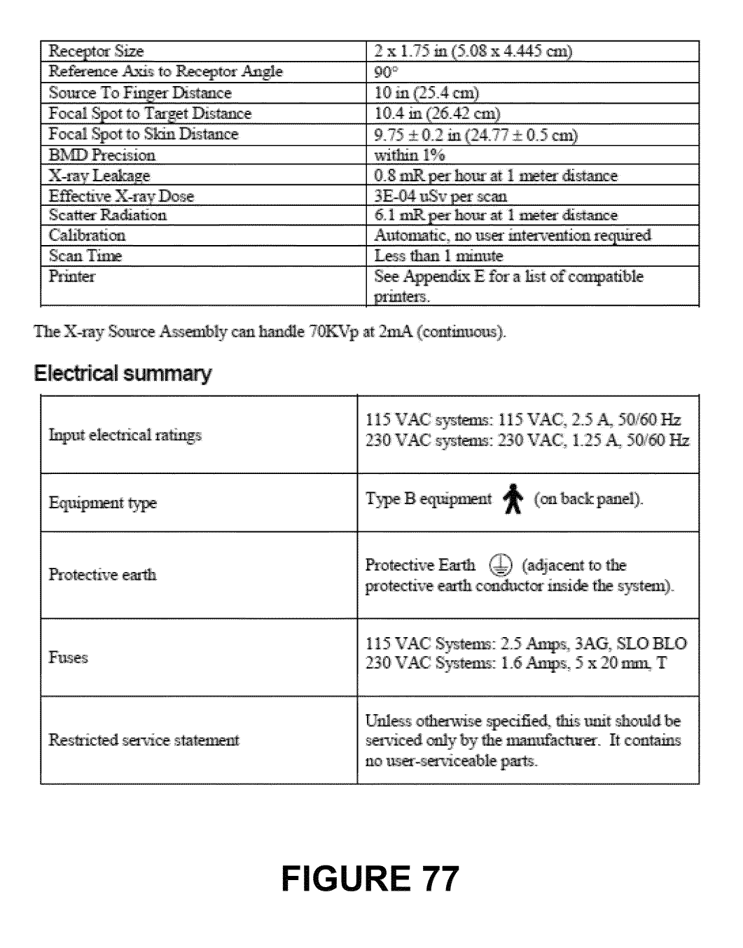

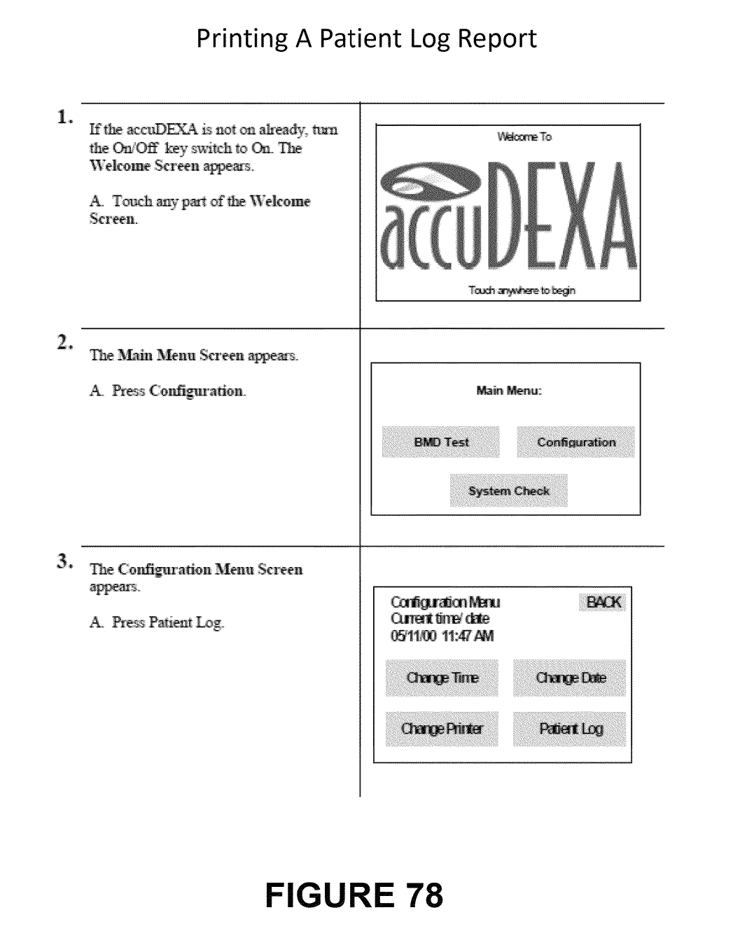

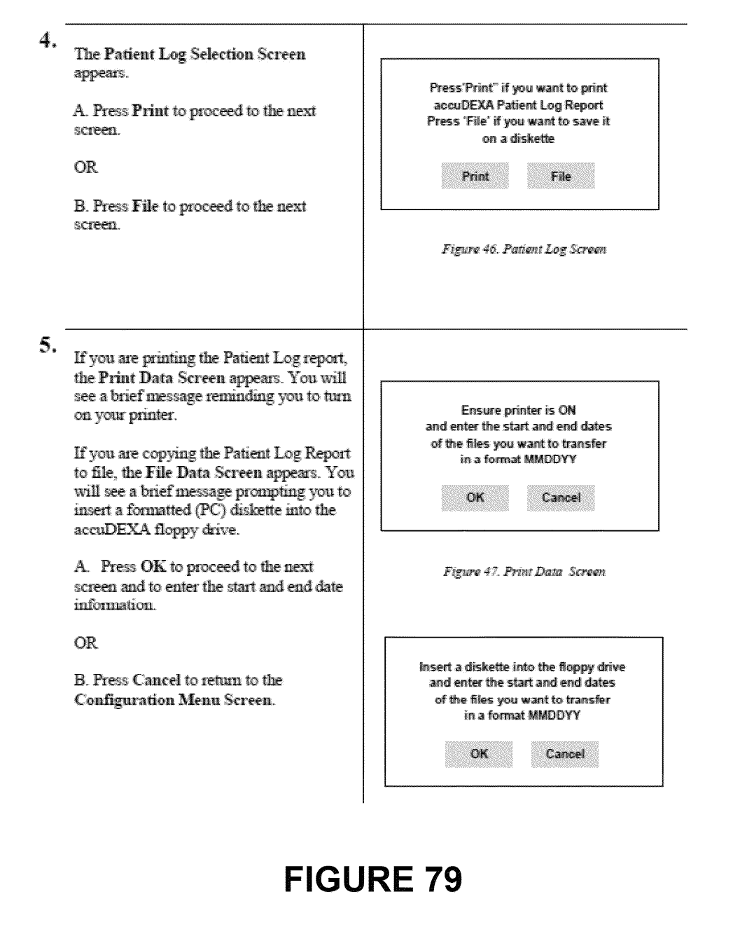

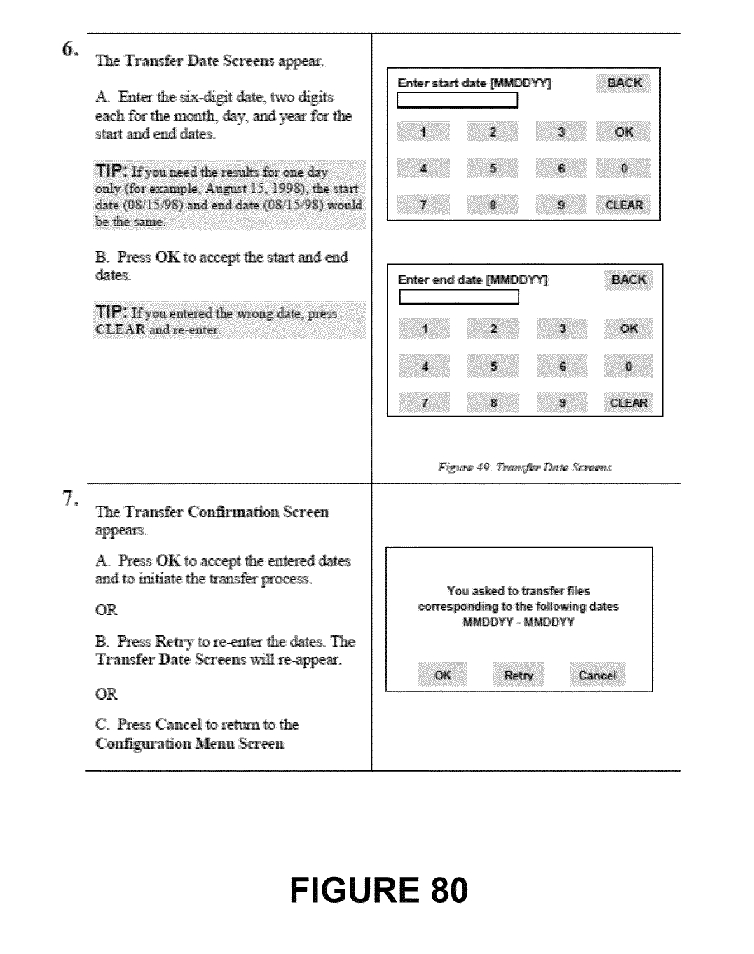

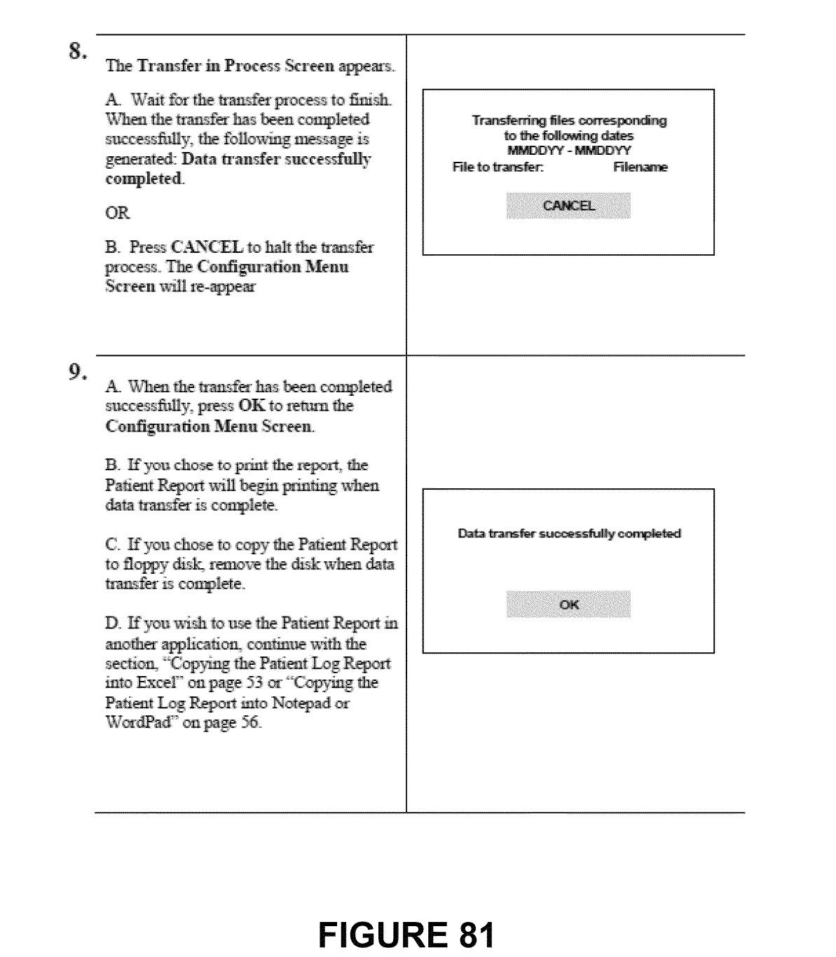

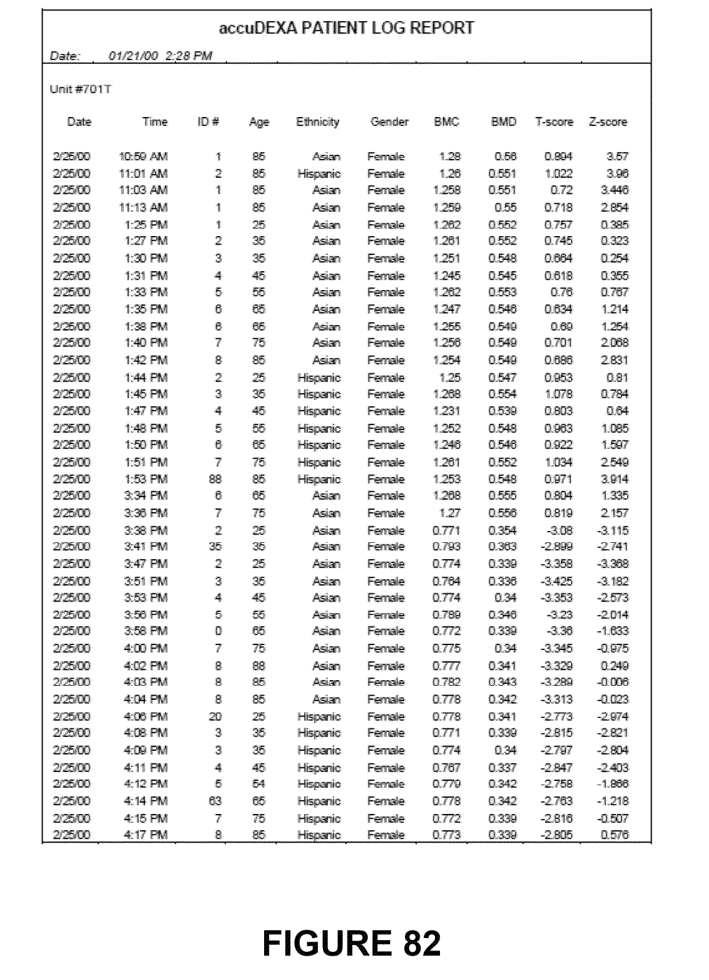

In an example configuration, the densitometer may be embodied in accordance with the example specifications and operate in accordance with the electrical summary depicted in FIG. 77 and FIG. 78.

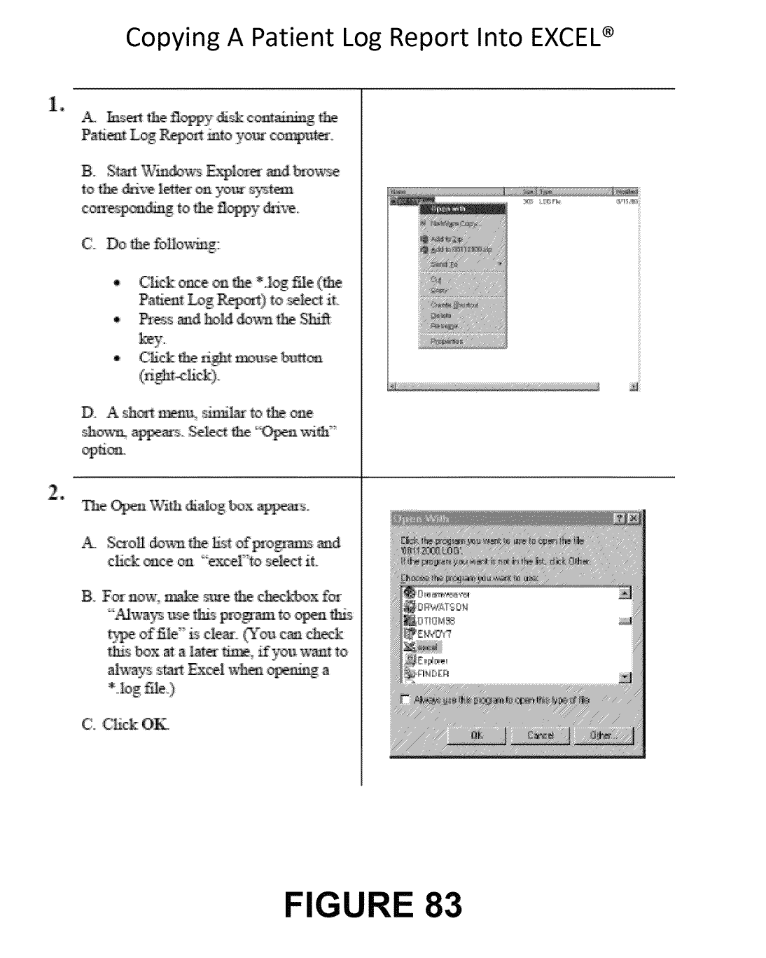

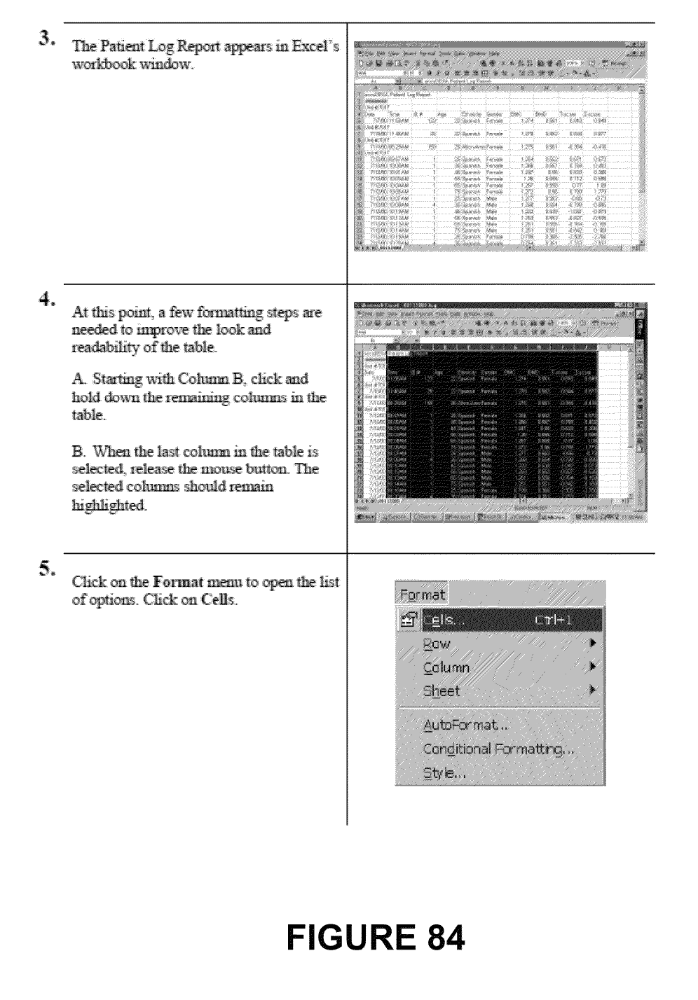

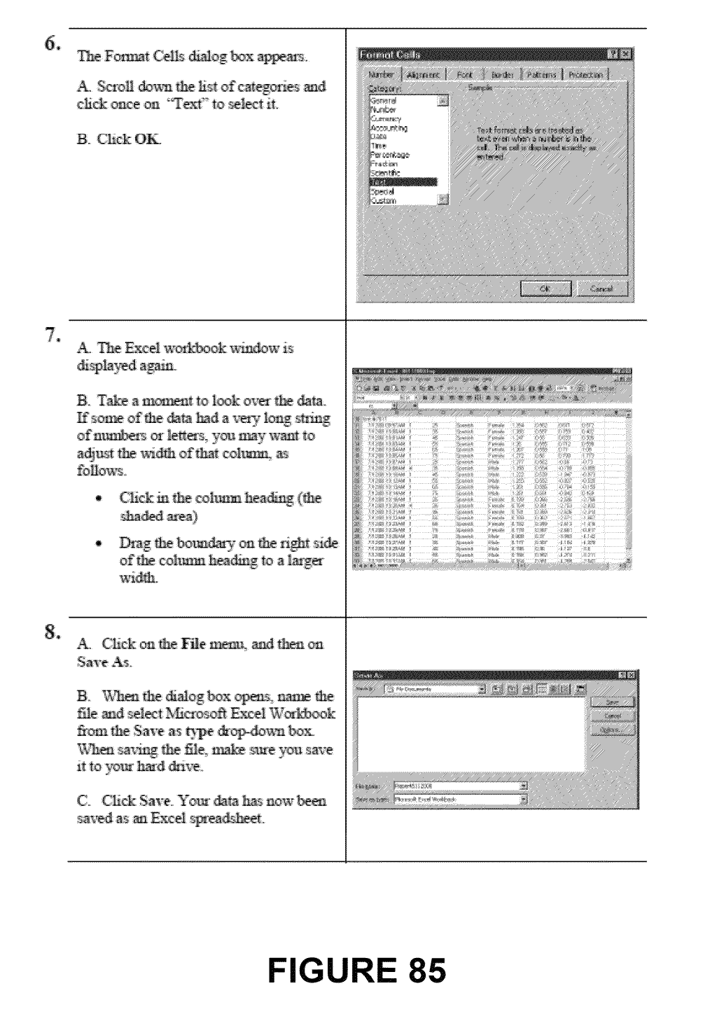

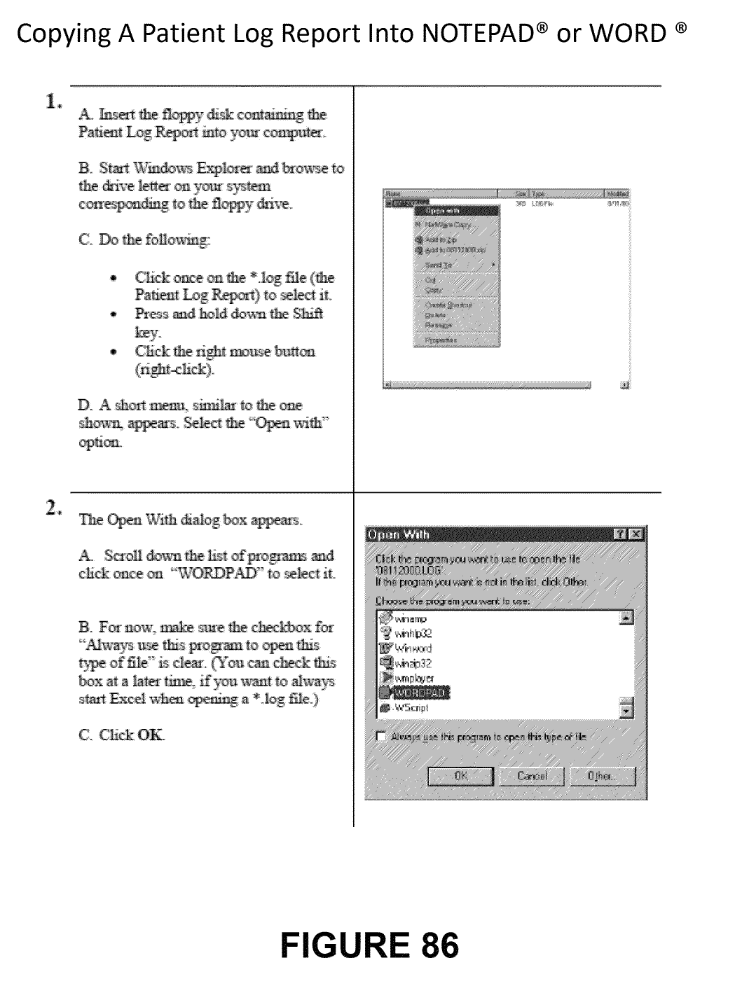

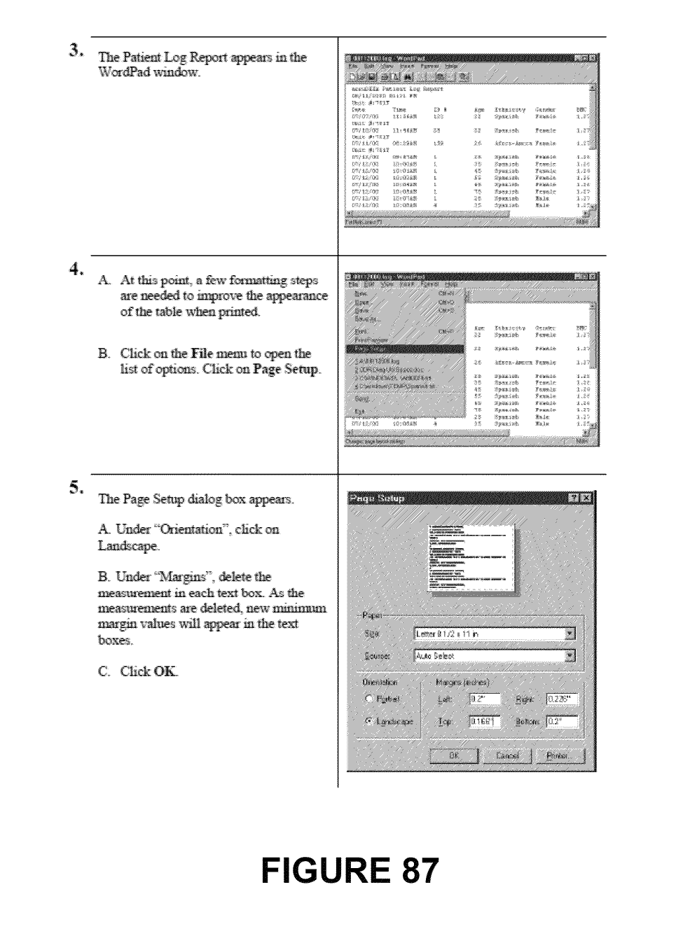

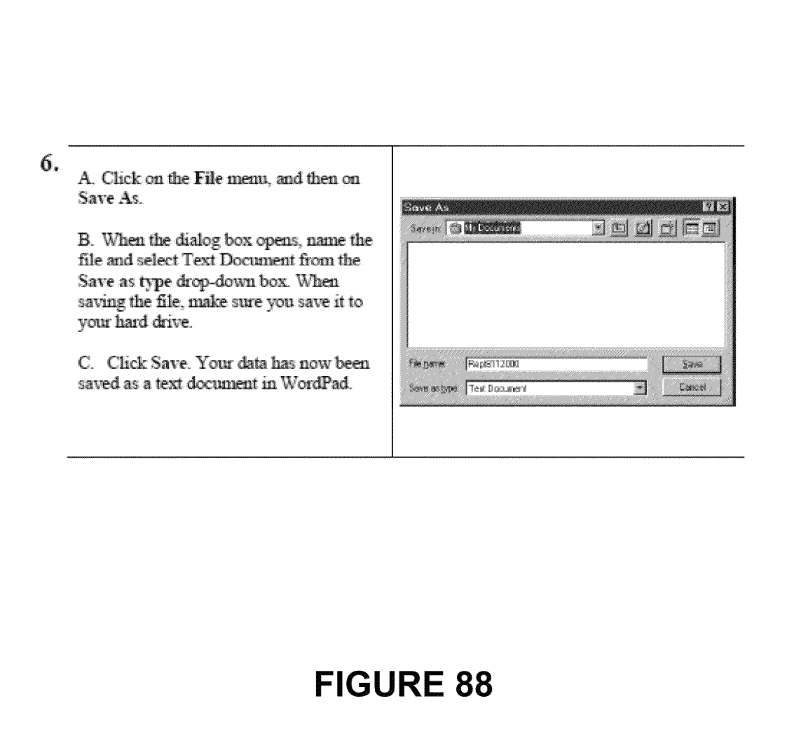

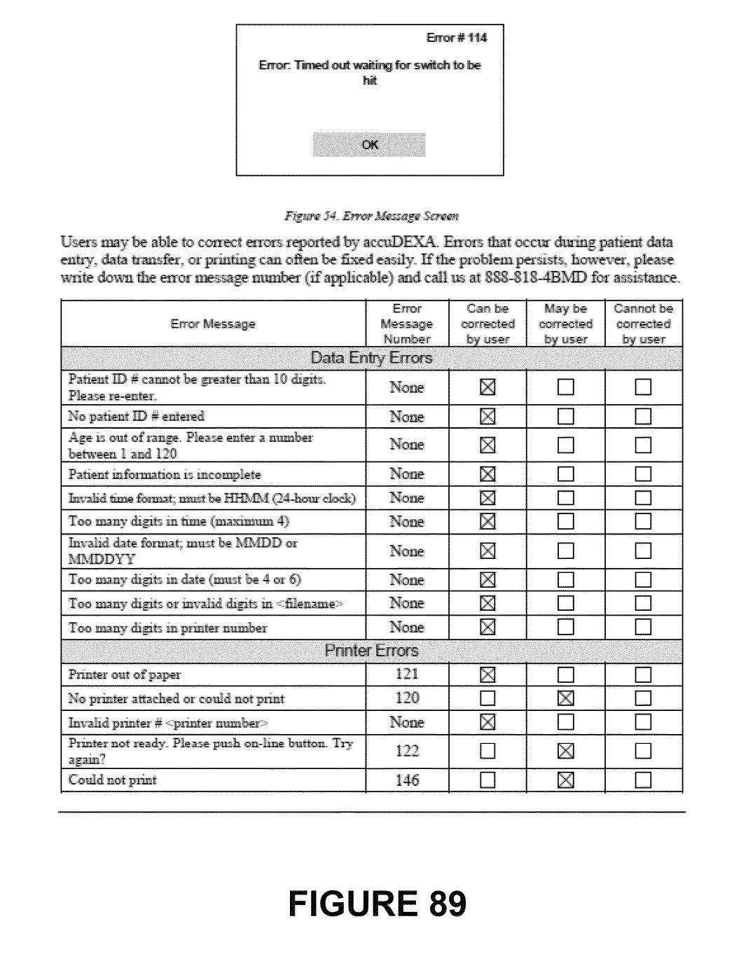

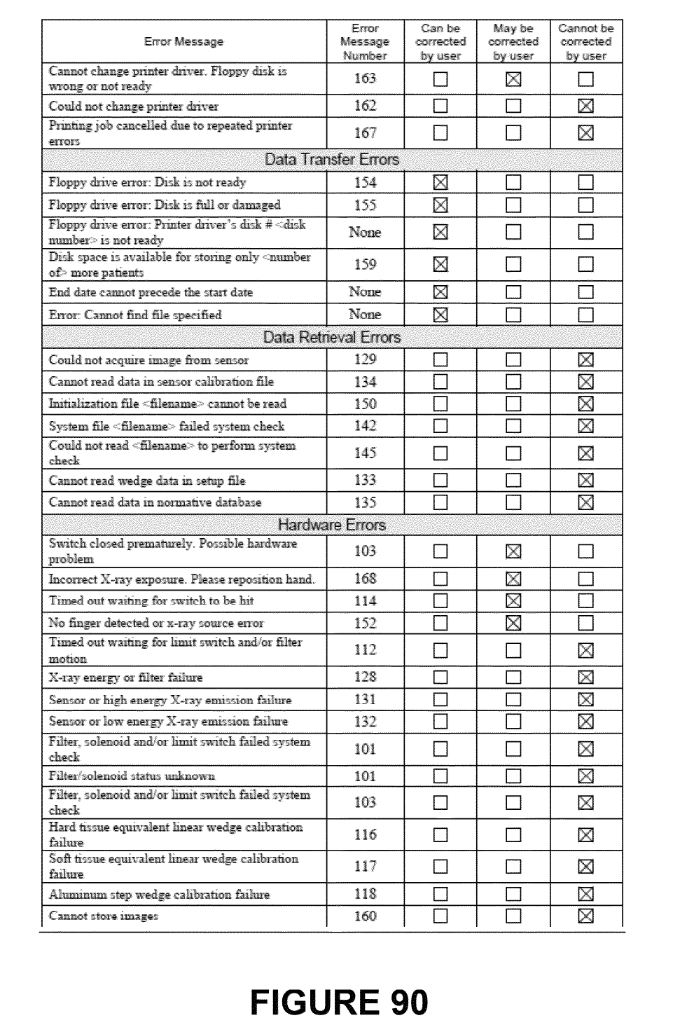

FIG. 79 through FIG. 83 illustrate an example process for printing a patient log report. A patient log report may comprise patient information, BMD and BMC scores, and/or t- and z-scores. (X-ray images and BMD report graphs are not included.). After performing the procedure, a single log file may be generated including test results from the range of dates (one day or many) specified by the user. FIG. 84 through FIG. 89 illustrate an example process for copying a patient log report. The patient log report may be copied into a spreadsheet, a document, file, or the like. The patient log report may be copied into any appropriate format, such as, for example, EXCEL®, WORD®, NOTEPAD®, or the like.

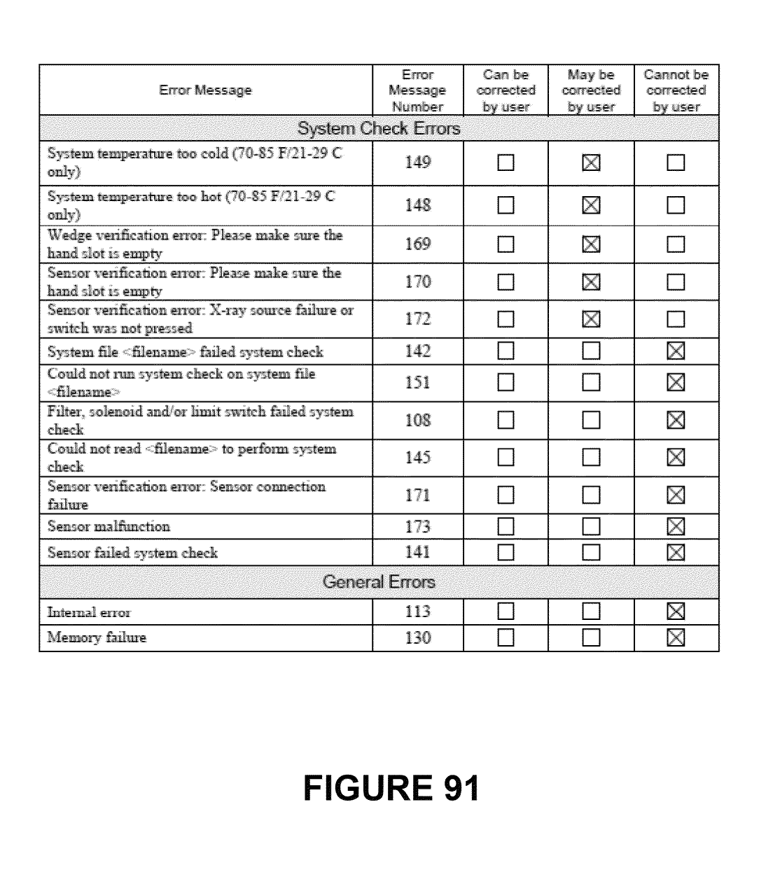

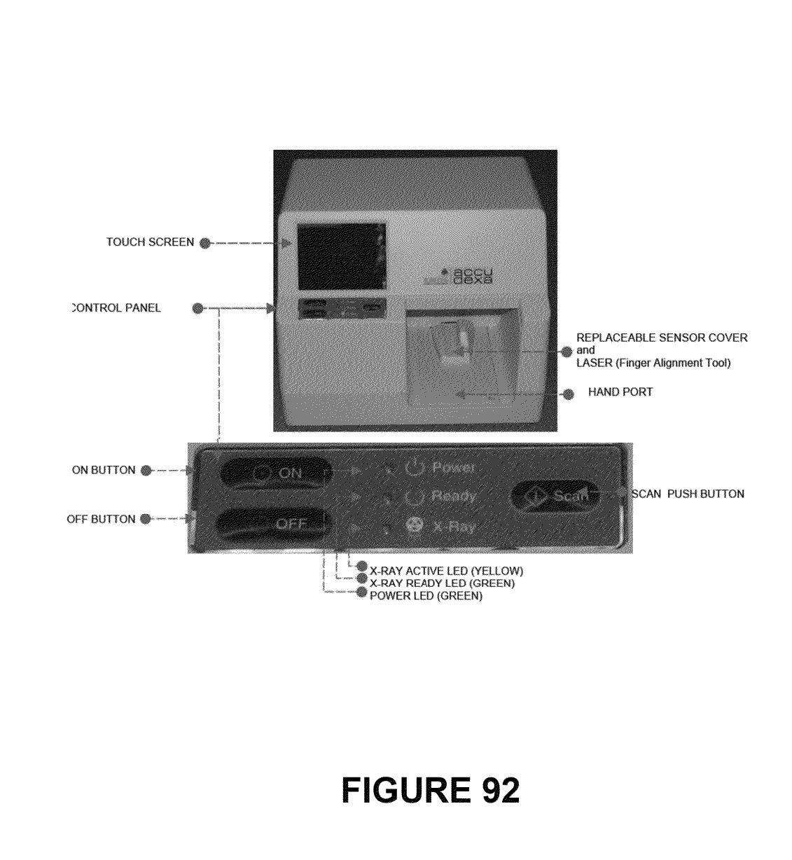

FIG. 90 through FIG. 92 depict example error messages.

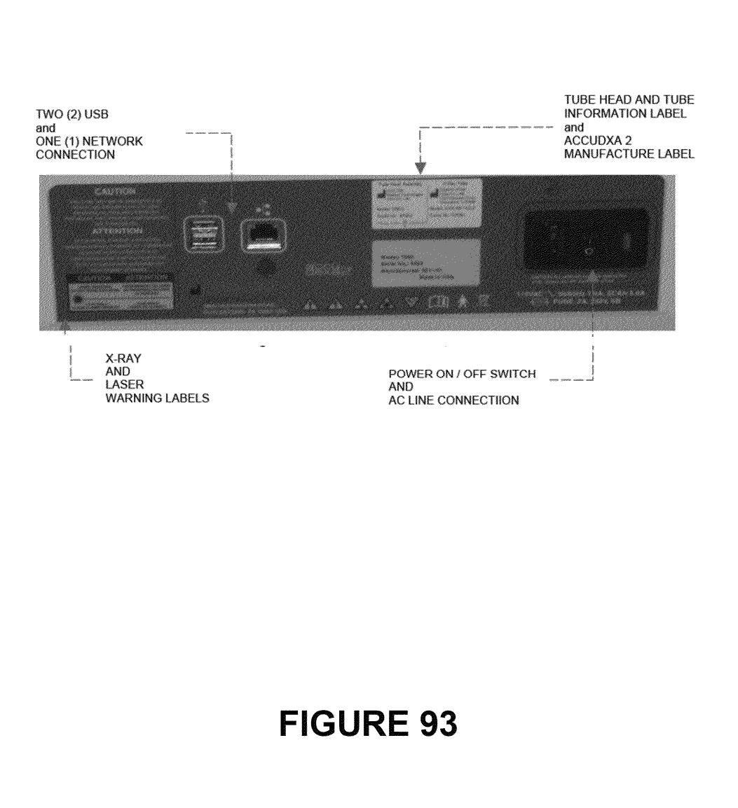

FIG. 93 is an example depiction of a front view of an example embodiment of the densitometer.

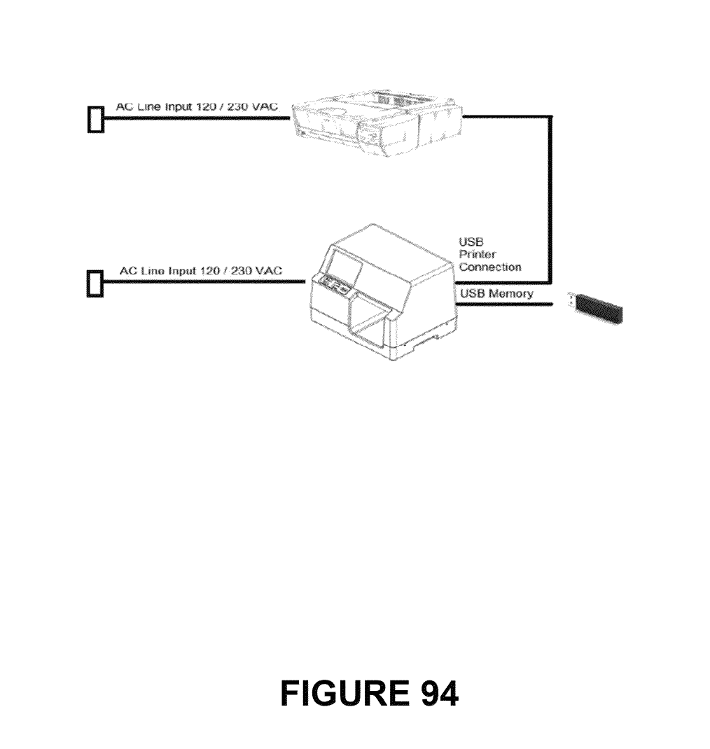

FIG. 94 is a depiction of a back view of an example embodiment of the densitometer.

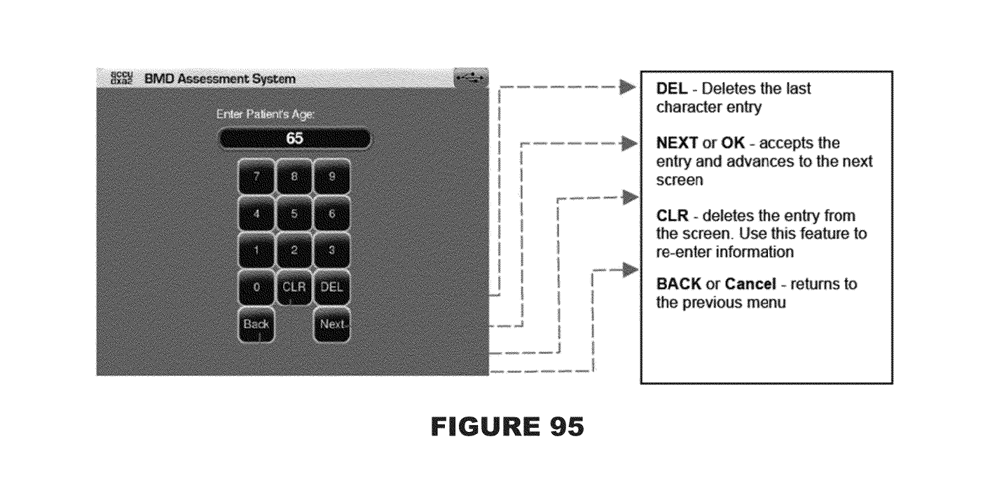

A printer or USB Thumb Drive may be installed in order to function with the densitometer as described below. FIG. 95 is a block diagram of an example configuration of the densitometer coupled to a printer and/or USB Thumb Drive.

In an example embodiment, information may be entered into the densitometer via a touch screen. An operator may enter information and may initiate a BMD test by using the touch-sensitive glass-on-glass color LCD screen. The touch screen may react to the contact of the operator's finger.

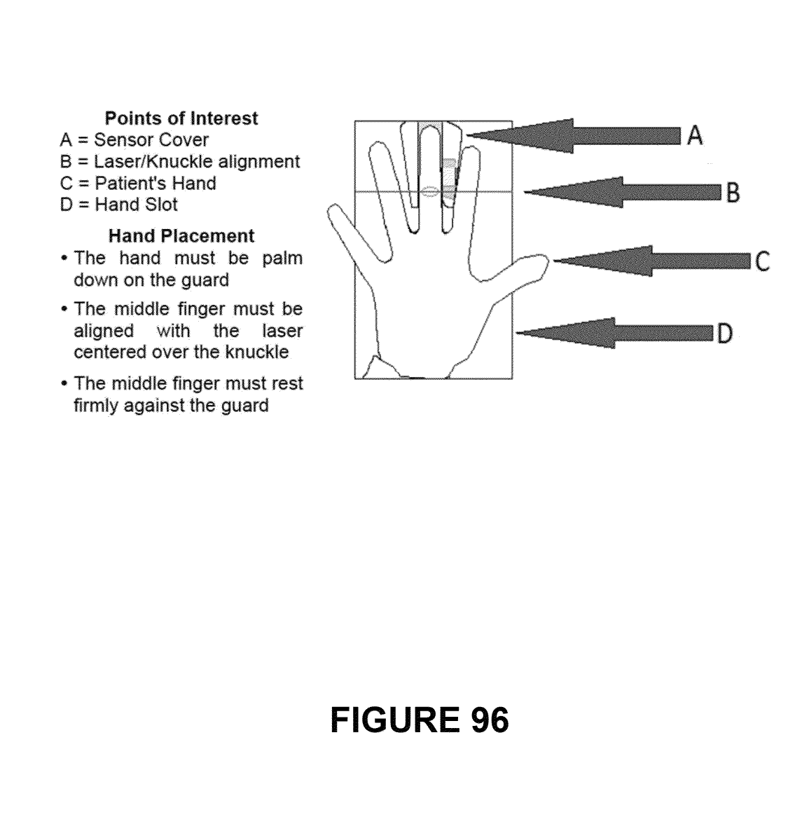

FIG. 96 is an example illustration of some of the on-screen features based on age of the Accudxa2® densitometer appear below using the Age.

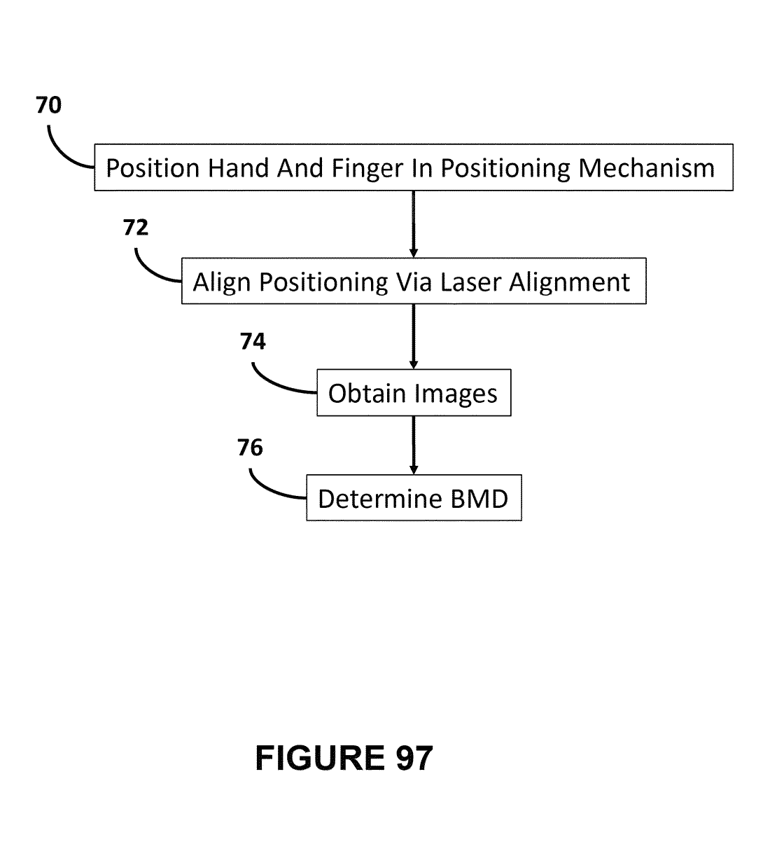

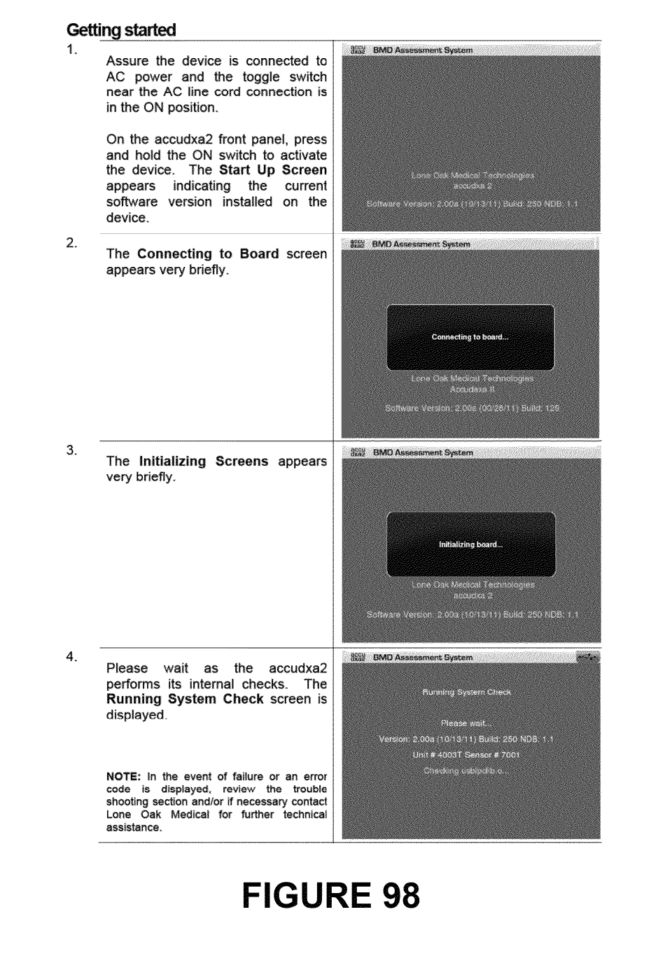

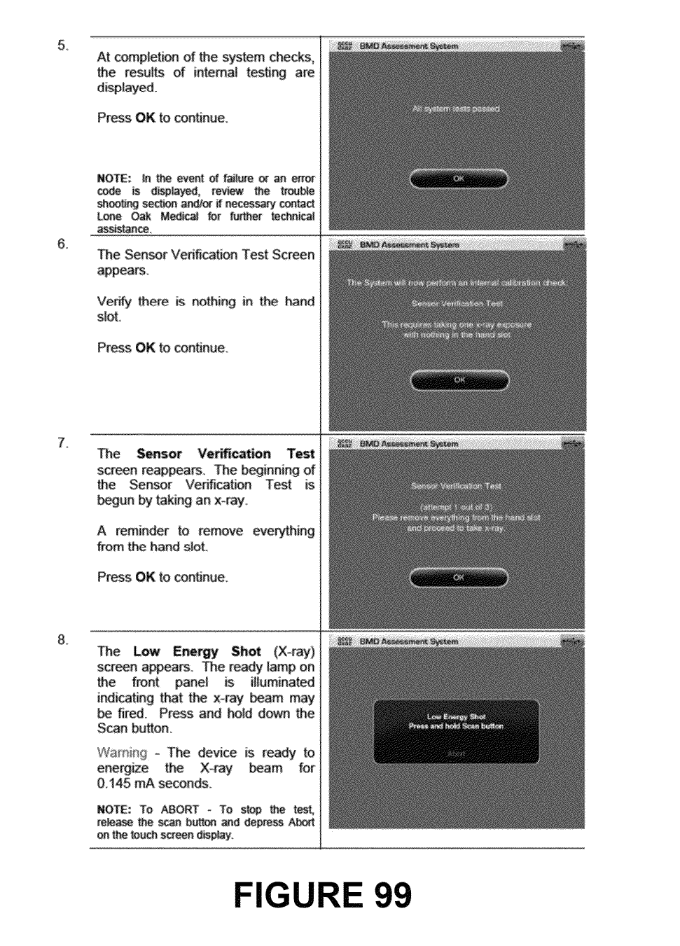

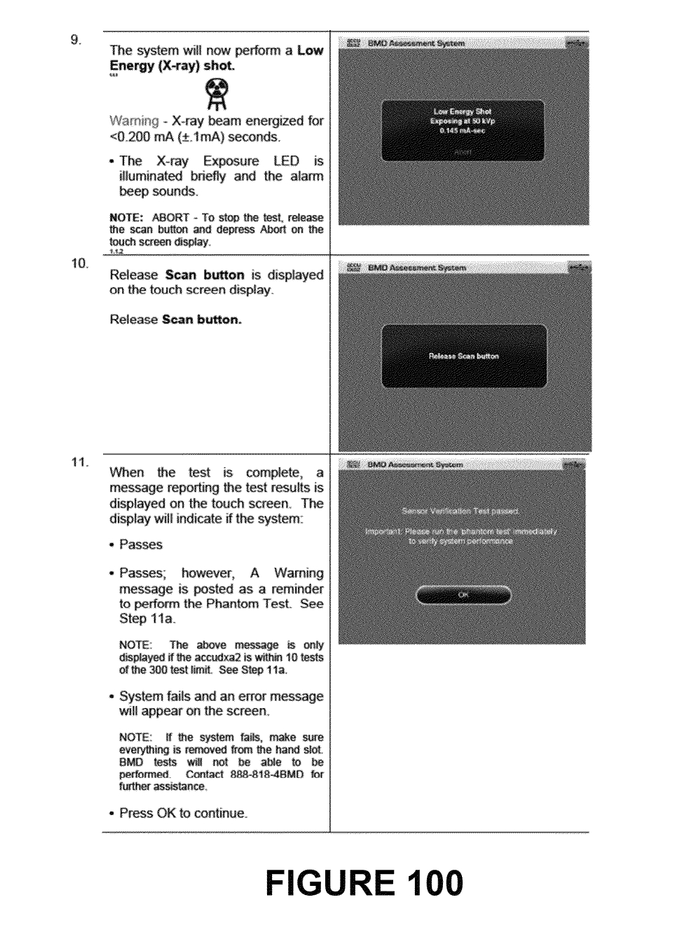

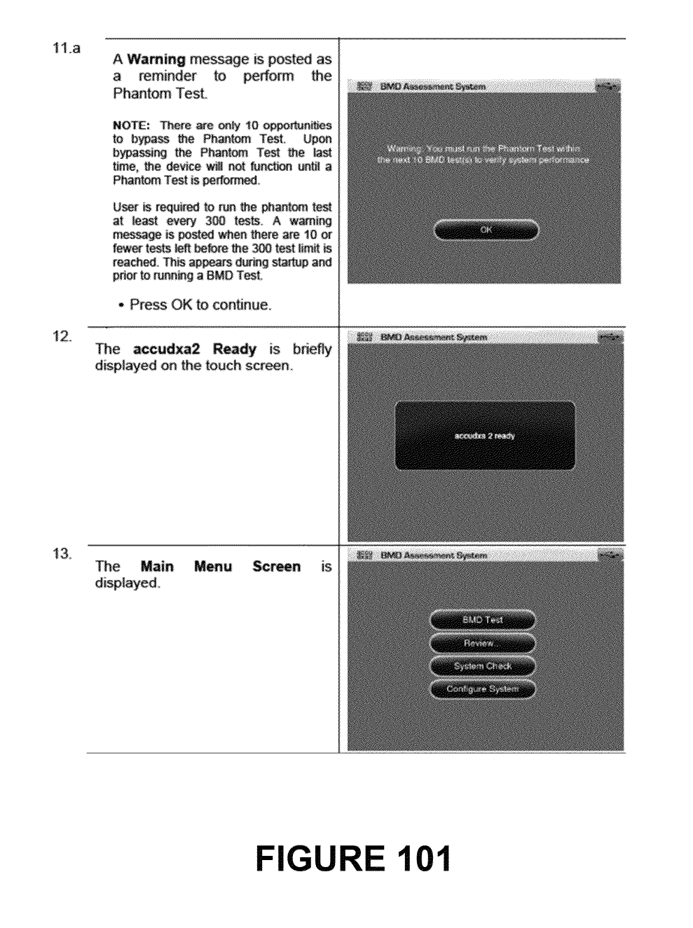

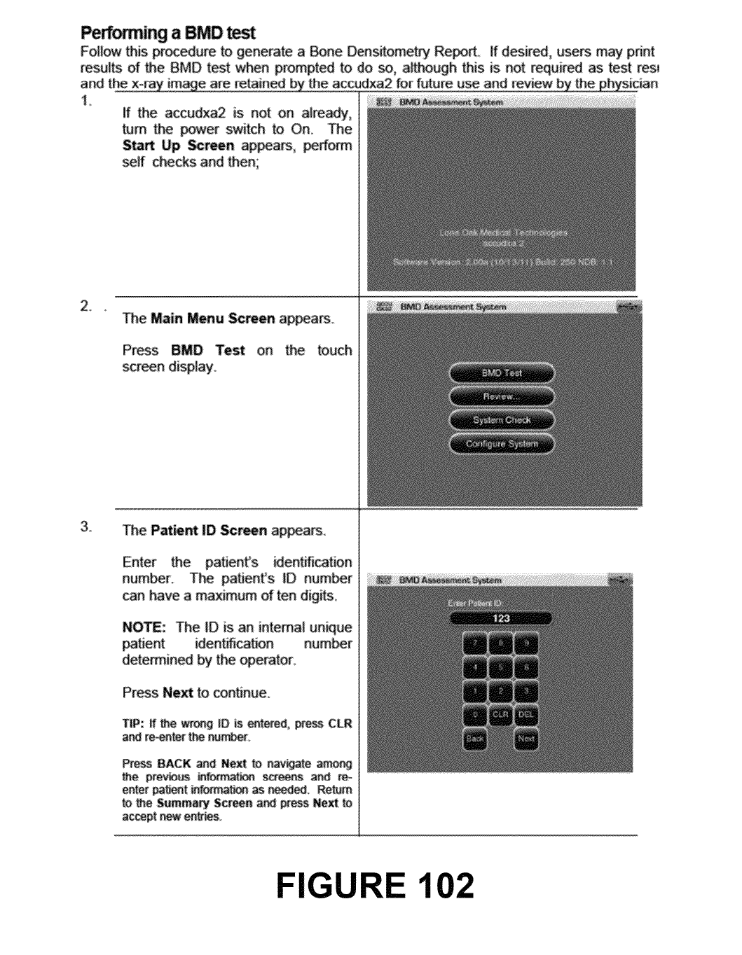

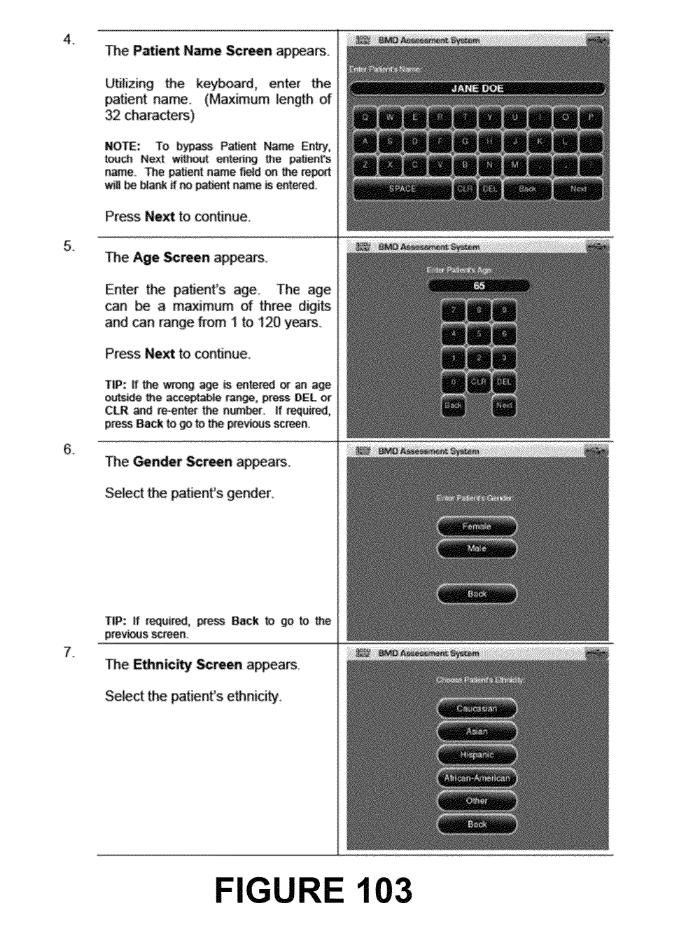

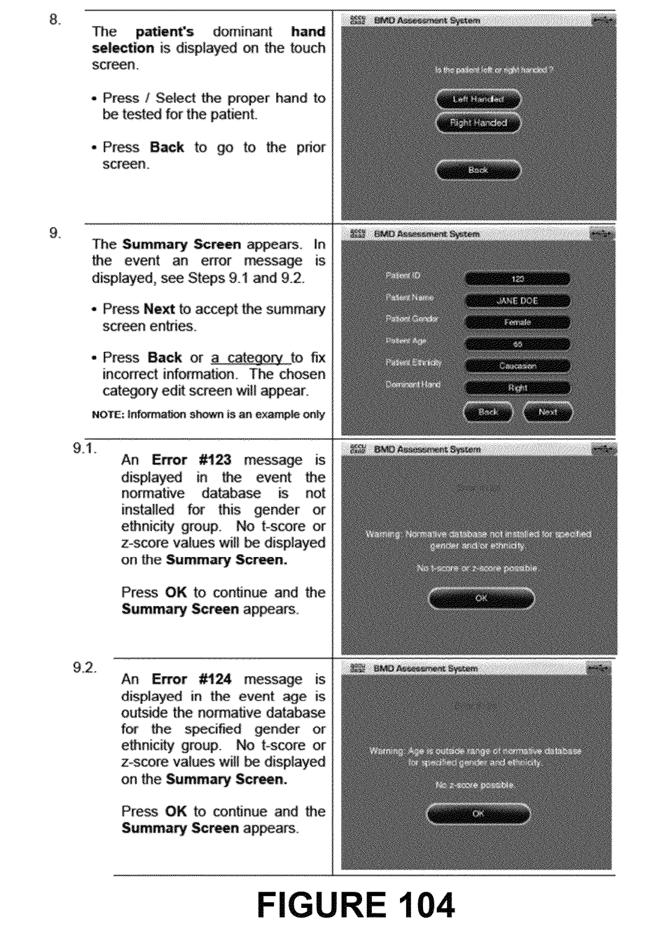

FIG. 97 depicts correct finger positioning for a BMD Test. FIG. 98 is a flow chart of an example process for positioning and BMD testing. To appropriately position a finger in the Accudxa2®, the patient's hand may be placed palm-down on the hand plate and the finger positioned in the positioning mechanism at step 70. A finger (e.g., the middle finger) may be aligned with the laser centered over the knuckle at step 72. The middle finger may rest firmly against the guide. To ensure proper finger placement/positioning, and to ensure accurate and precise BMD test results, all hand and wrist jewelry should be removed. Removing jewelry may improve finger positioning, increase patient comfort, and help the patient to remain still during the procedure. Incorrect positioning or finger movement during testing may lead to inaccurate test results.

If jewelry cannot be removed, extra care should be taken to ensure correct positioning. For example, a ring may prevent a patient from resting his/her finger against the finger guide. As long as the finger placement approximates the description provided herein, and the X-ray image contains no part of a ring or jewelry, the exam may be valid.

In order to obtain successful BMD test results, the operator may follow several simple guidelines. The patient's hand may be positioned palm down and held motionless throughout the exam. During an exam, the Accudxa2® densitometer may rest on a table roughly 30 inches from the floor. Patients may be in a comfortable position during the BMD Test. The patient's seat may be stationary and approximately 18 inches from the floor. The Accudxa2® densitometer may be operated within predetermined temperature and humidity ranges.

Images may be obtained at step 74. In an example embodiment, the operator may ensure that an audible signal is heard for each of the three X-ray exposures that occur during the BMD test, the radiation label is affixed and visible on the rear panel of the densitometer and a small indicator (X-ray Exposure Light) is illuminated during each exposure, and the Accudxa2® densitometer performs a system check each time the device is powered on. The software may also perform an internal calibration before the X-ray exposures are taken and before the BMD values are calculated. If the system check or the internal calibration is unsuccessful, an error message may be displayed on the LCD screen. If the problem cannot be corrected, the error message number may be noted and assistance may be obtained by referencing the error message number.

Note, during BMD tests, the Accudxa2® densitometer may verify X-ray exposures as they are taken. This verification calculates the difference between high and low energy exposure to ensure that only X-rays taken at the correct energy and exposure times are accepted. The obtained images may be used to determine bone mineral density (BMD) as described herein at step 76.

FIG. 98 through FIG. 109 depict an example process for using the Accudxa2® densitometer.

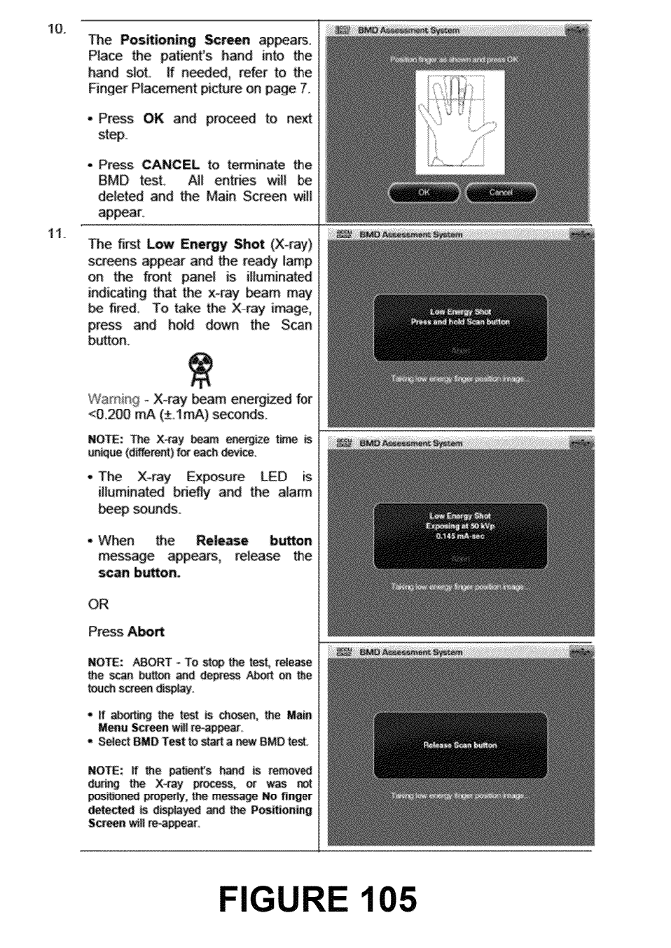

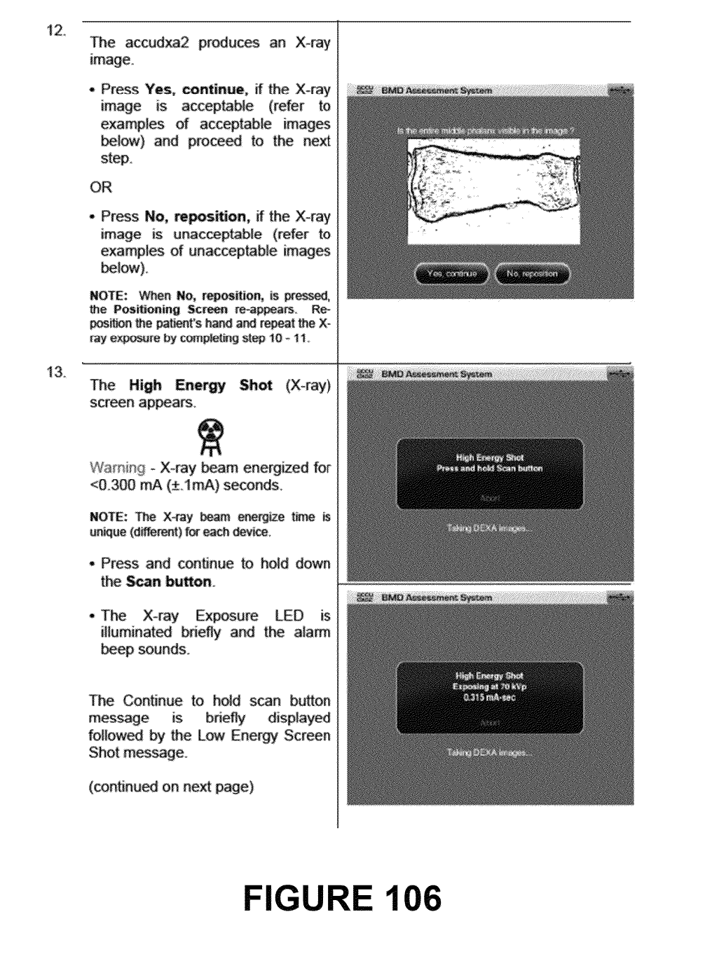

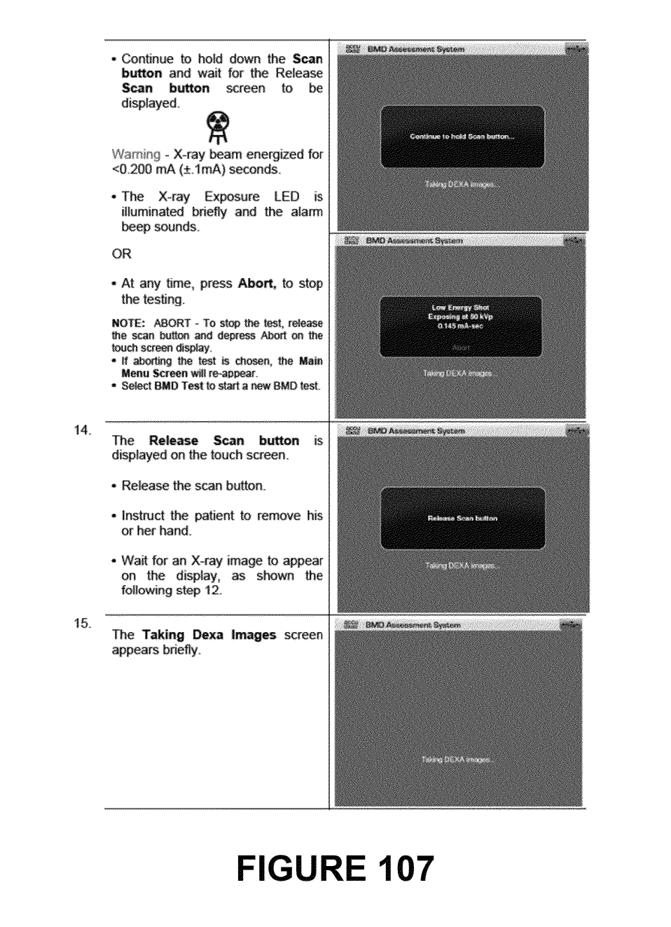

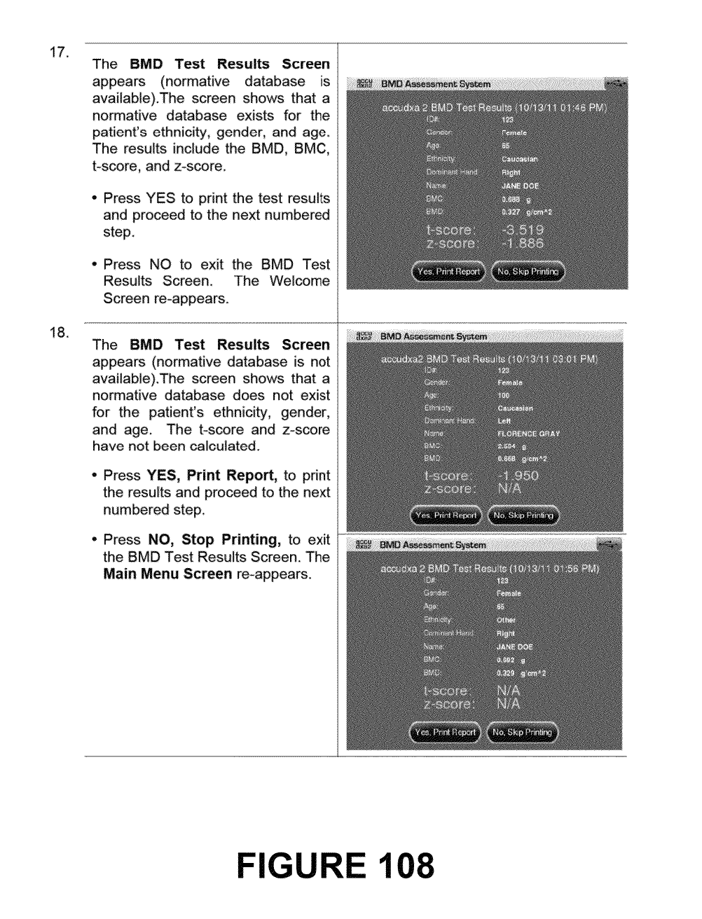

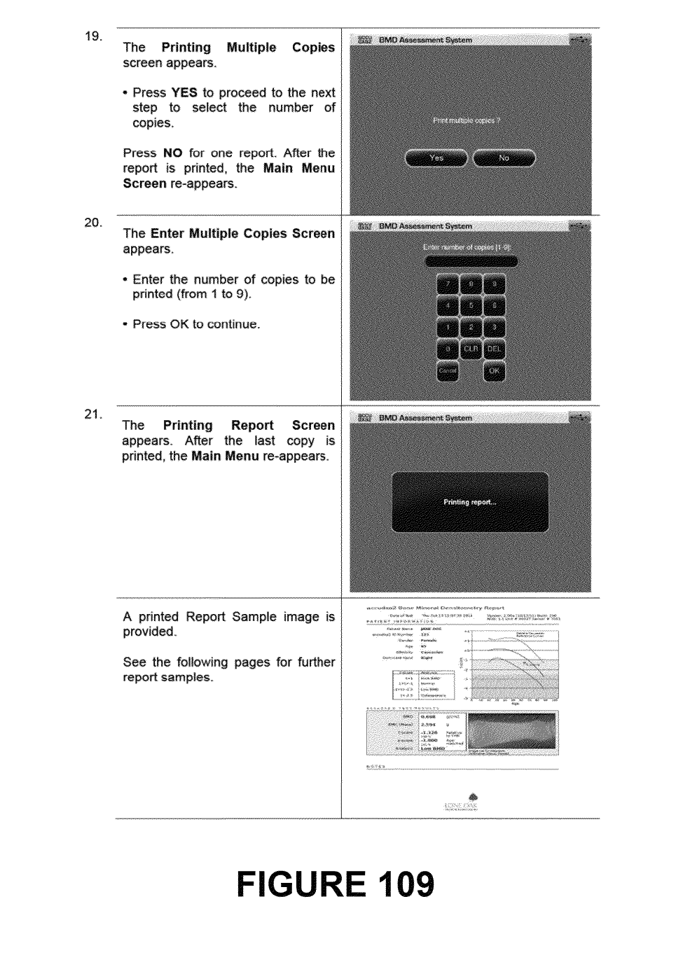

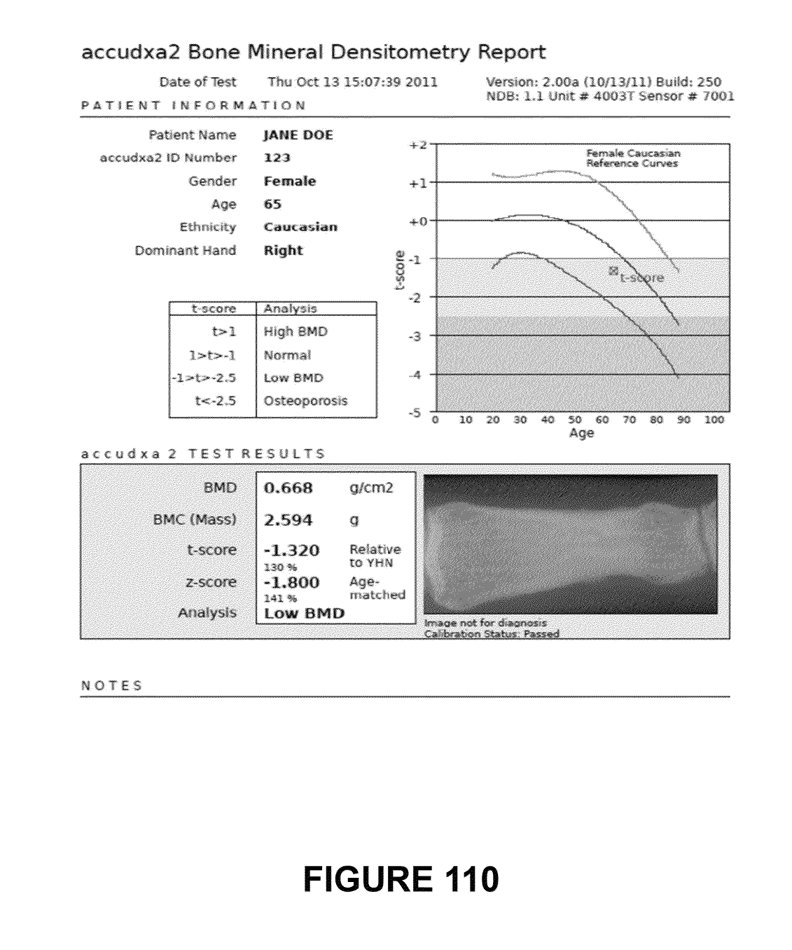

FIG. 110, FIG. 111, and FIG. 112 show examples of bone densitometry reports. The reports in FIG. 110, FIG. 111, and FIG. 112 share some common features, including general report information (report date and time, software version, and device serial number), patient information (Patient ID, Gender, Age, Ethnicity and Dominant Hand), and BMD test information (X-ray image area and BMC and BMD results). There also are some report differences as described below.

In FIG. 110 a patient's BMD results were compared with an available normative database. The t-score was calculated from the BMD results of the patient and a database population matching the patient's gender and ethnicity. The z-score was generated using those same parameters (gender and ethnicity) and the patient's age.

In FIG. 111 a patient's BMD results also were compared with an available normative database. In this report, however, the z-score was not calculated because the patient's age (95) was “out of range” and could not be matched with an equivalent age in the database. A warning note is printed on the report.

In FIG. 112 a patient's BMD results were generated but were not compared to a database that matched the patient's ethnicity and gender. Instead, the report graphs the results using reference curves based on the Caucasian database for the same gender and prints a cautionary note on the report.

The formulas depicted in FIG. 55 may be used by the Accudxa2® densitometer to calculate t-scores, z-scores, and to provide, as a percentage, where those scores lie in relation to the mean BMD. The analysis may be calculated automatically, based on t-score, and reported as Normal, Osteopenia, or Osteoporosis.

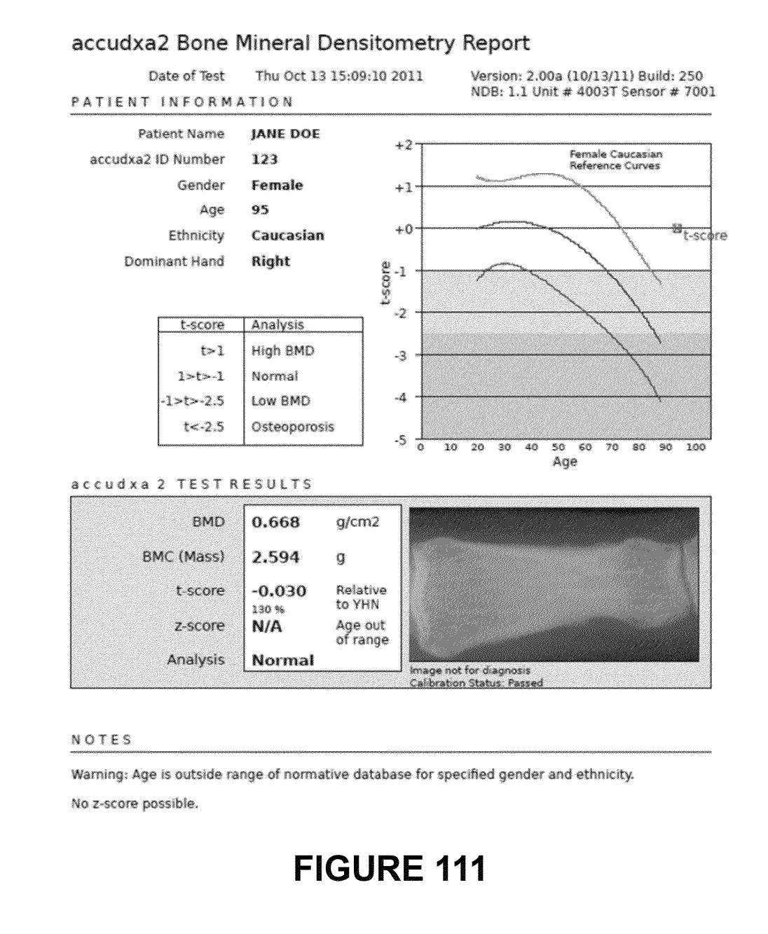

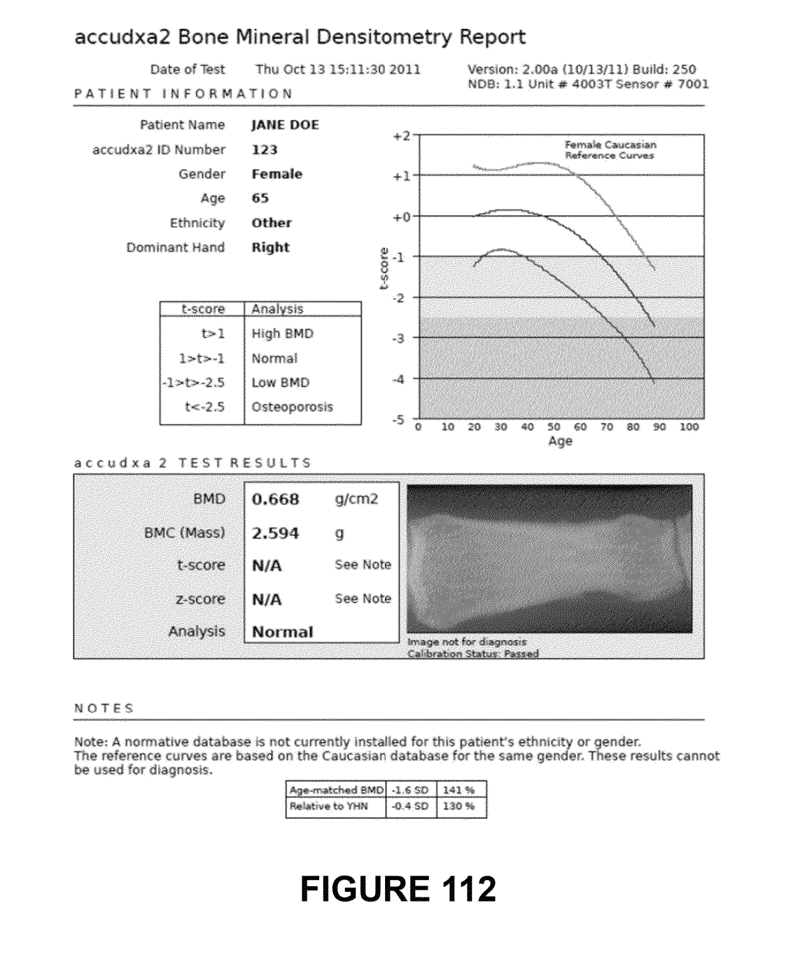

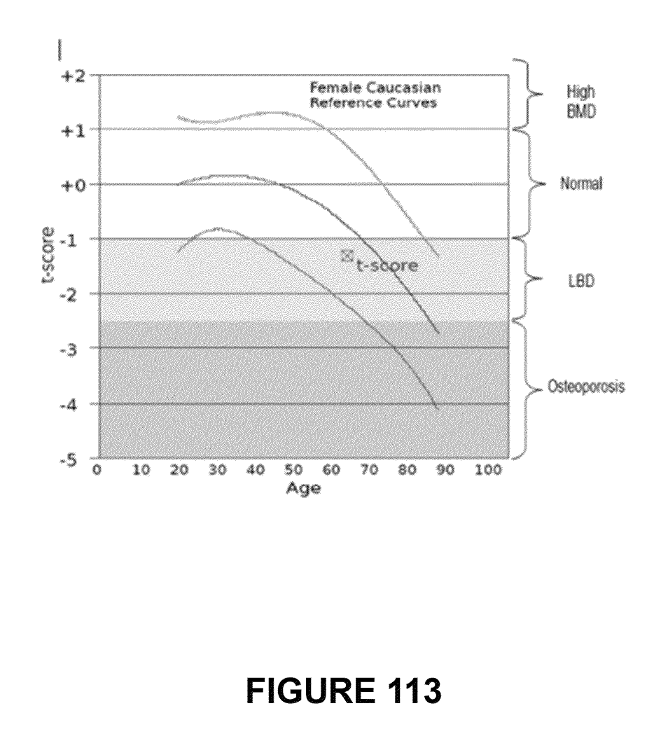

FIG. 113 depicts sample graphs of t-scores versus age. On the sample reference curve shown in FIG. 113, the scale of t-scores is shown at the left and the scale for age is at the bottom. The three curved lines are isometric z-scores. The top curve represents one standard deviation above the age-matched mean BMD. The middle curve represents the age-matched mean BMD. The bottom curve represents one standard deviation below the age-matched mean BMD. Isometric t-scores are displayed on the y-axis. The t-scores can be positive or negative and correspond to standard deviation increases or decreases in BMD as compared to a young, healthy normal (YHN) individual. The range of ages for z-scores is displayed on the x-axis. The t-score and z-score for the scanned patient can be seen graphically on the curve, and is represented by a small square box with a cross in it. In this example the patient has a lower than mean BMD compared to a young healthy normal (t-score) and age-matched (z-score) database.

Bone mineral estimates may be used to provide an index of fracture risk. Individuals who fall below the range of young healthy normal individuals may be at a greater risk for fracture. The World Health Organization (WHO) has established four general diagnostic categories that define categories for low bone density as shown in the table below.

| | |
|---|---|
| Normal | A value for bone mineral density (BMD) or bone |
| | mineral |
| | content (BMC) within 1 standard deviation (SD) |
| Low Bone | A value for BMD or BMC more than 1 SD below the |
| Mass | young |
| (osteopeniaor | |
| LBD) | |
| Osteoporosis | A value for BMD or BMC of 2.5 SD or more below the |
| | young adult mean. |
| Severe | A value for BMD or BMC more than 2.5 SD below the |
| Osteoporosis | young adult mean in the presence of one or more |
| | fragility fractures. |

The Accudxa2® densitometer may automatically calculate a patient's risk based on the t-score and may report the results as Normal, Low Bone Density (LBD), or Osteoporosis.

While low BMD may be a factor in determining a patient's risk for fracture, there may be other factors that also contribute to risk. Patients with a combination of several risk factors are at an increased risk of fracture. The following is a summary of risk factors.
Being female
A small, thin frame
Advanced age
A family history of osteoporosis
Early menopause
Abnormal absence of menstrual periods (amenorrhea)
Anorexia nervosa or bulimia
A diet low in calcium
Use of certain medications (steroids, anticonvulsants, excessive thyroid hormones,
certain cancer treatments)
Low testosterone levels in men
A sedentary lifestyle
Cigarette smoking
Excessive alcohol intake
Malabsorption problems

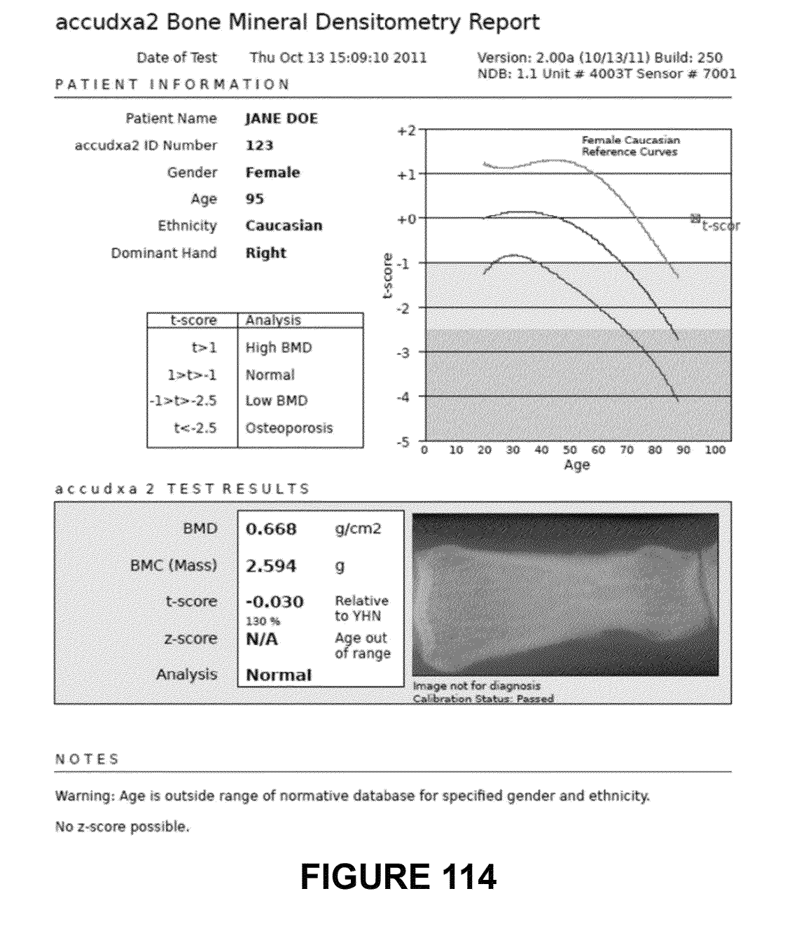

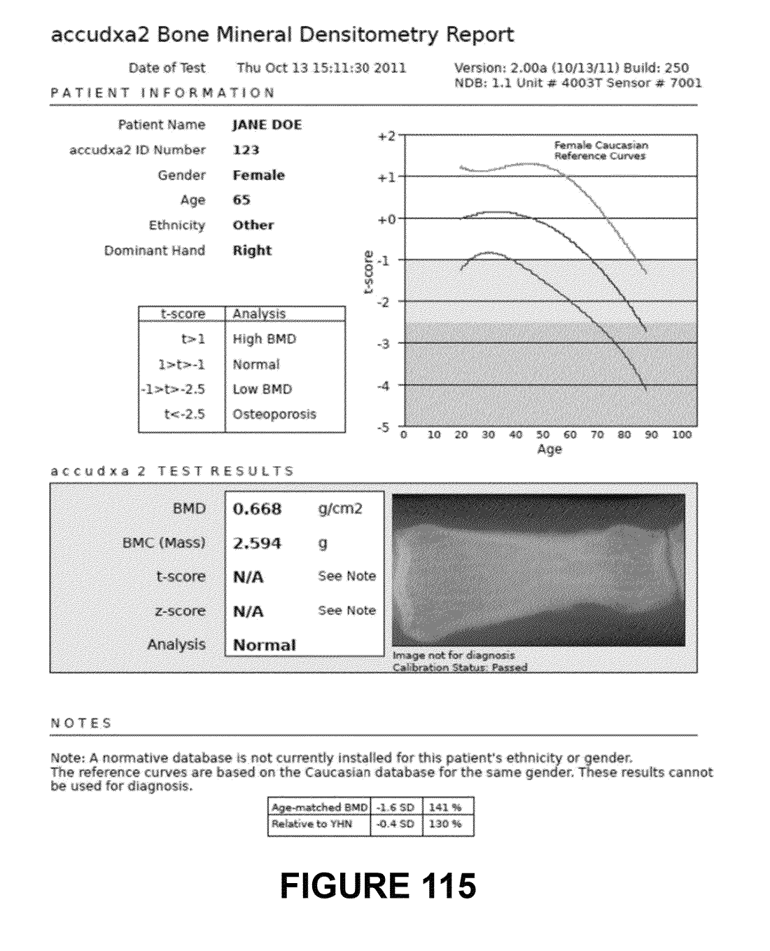

FIGS. 114 and 115 illustrate example densitometry reports.

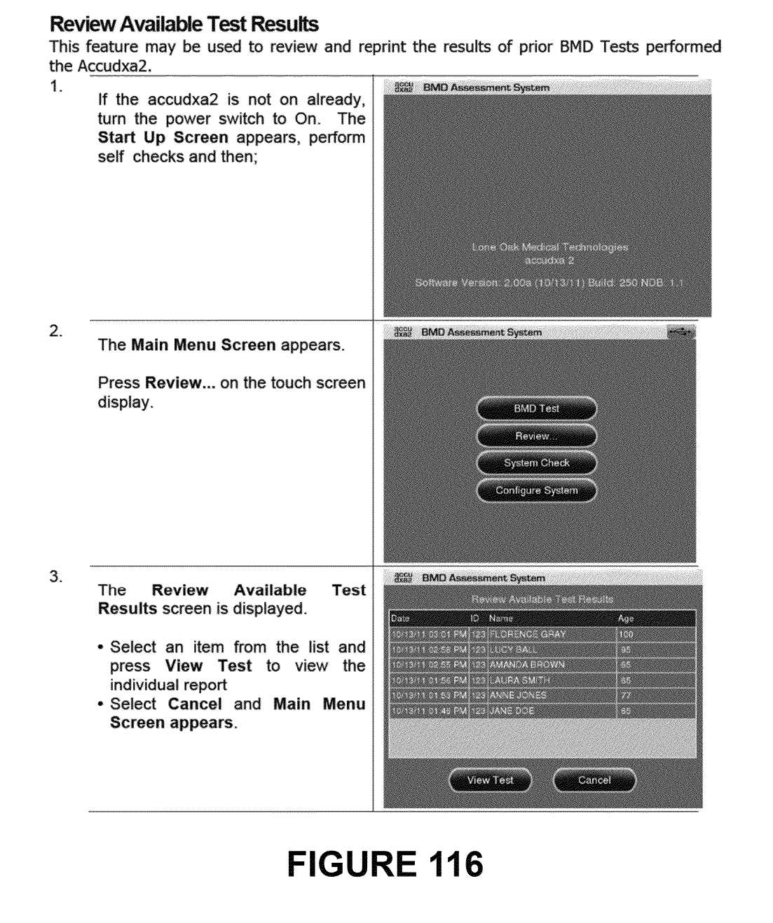

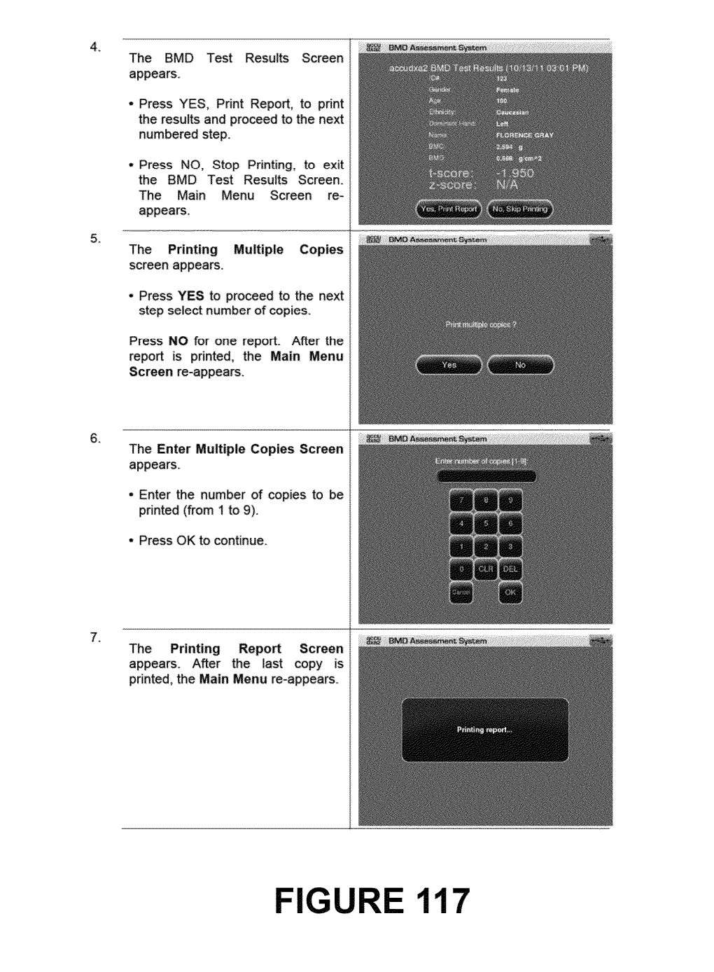

FIGS. 116 and 117 depict an example process for reviewing stored BMD Test Reports on the glass-on-glass color LCD and/or an externally connected printer.

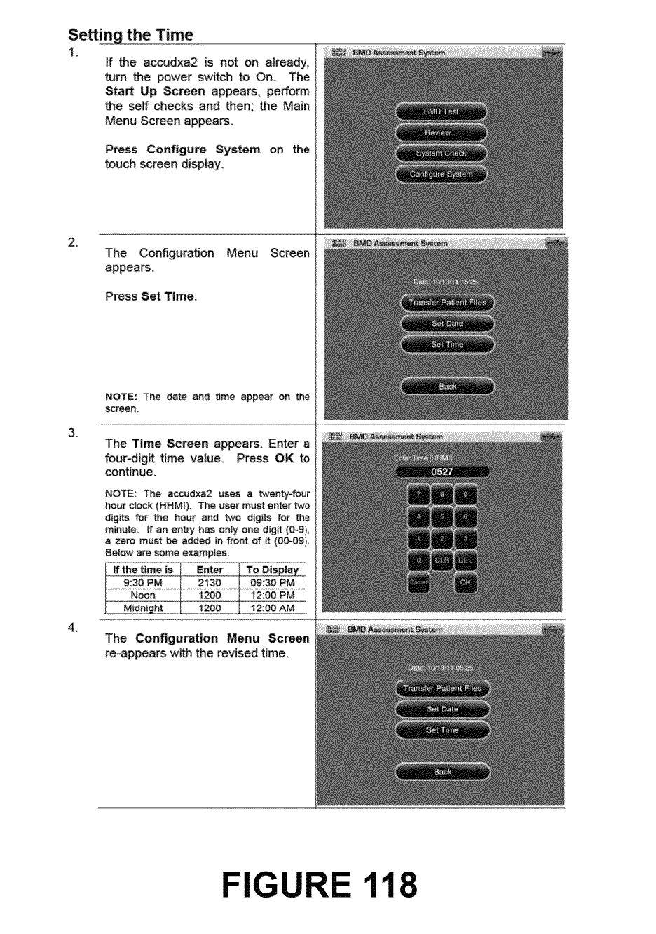

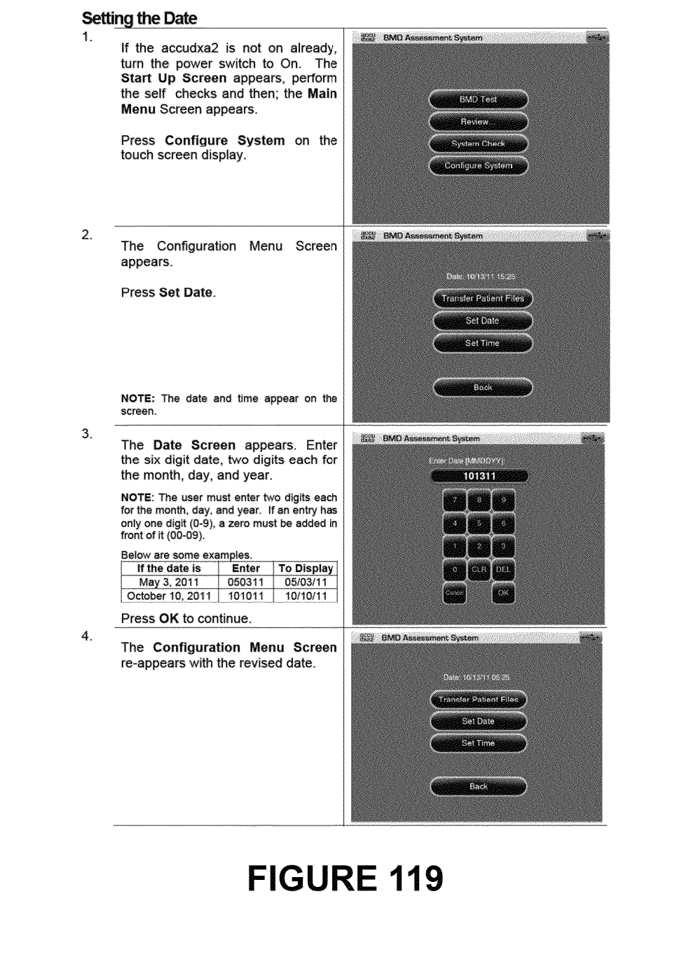

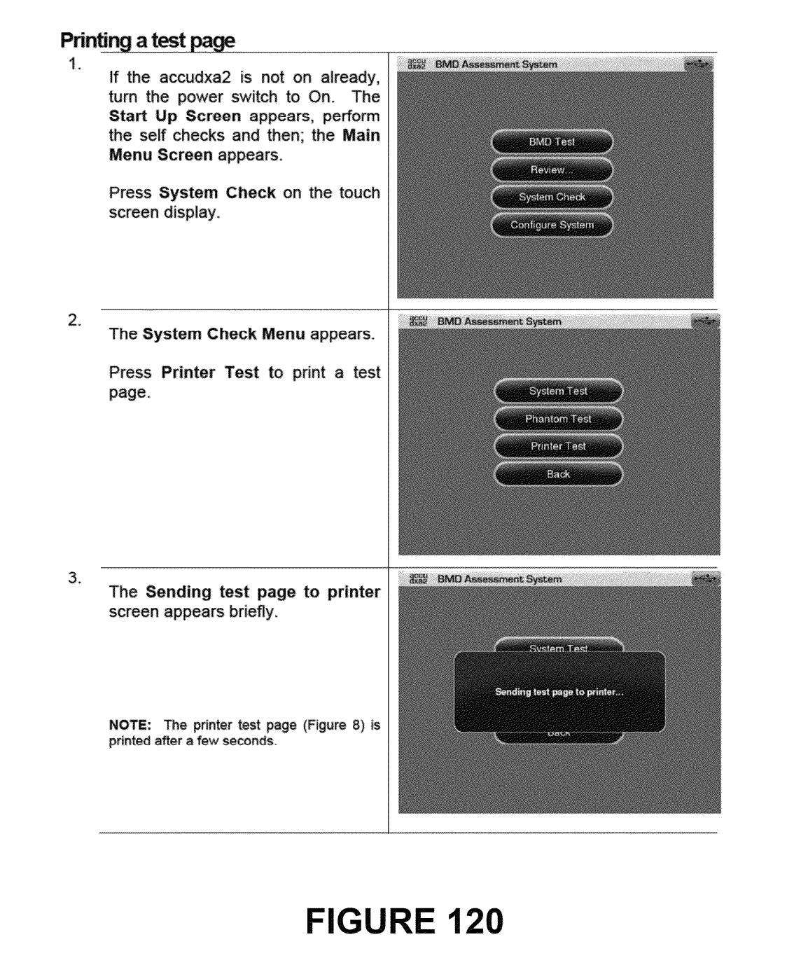

FIGS. 118 and 119 depict an example process for setting the date and the time stored in the processor of the Accudxa2®.

FIG. 120 illustrates an example process for using the Accudxa2® to print a test report on an externally connected printer.

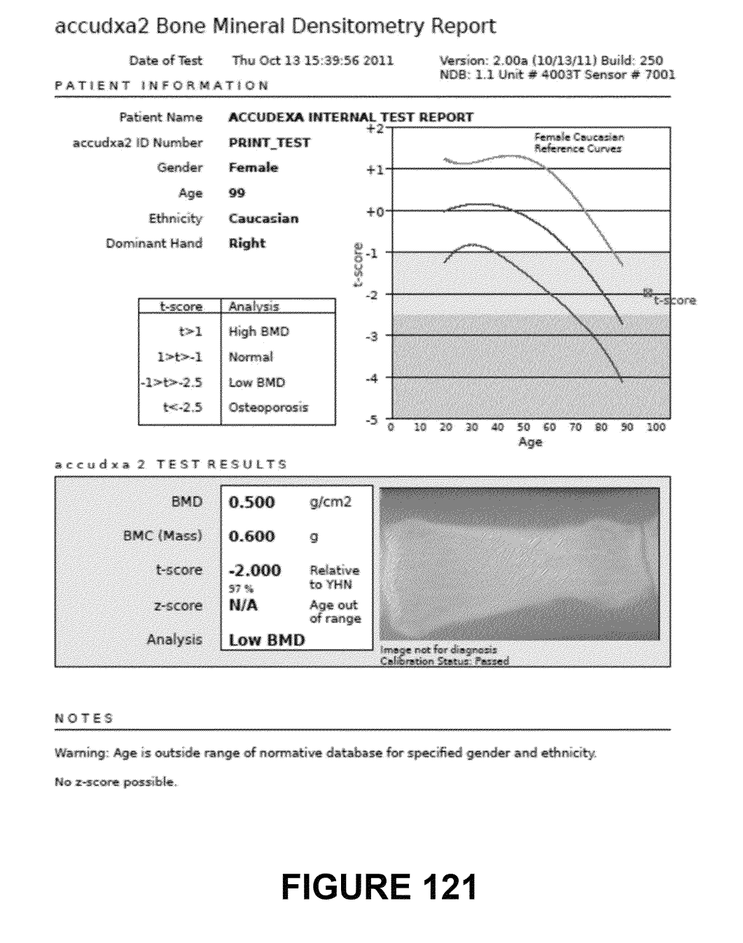

FIG. 121 illustrates an example of a test report printed on the Accudxa2® using an externally connected printer.

Phantom tests may be performed utilizing the densitometer. A phantom test comprises a quality-control check of the Accudxa2® densitometer system. It utilizes a finger phantom (article with known characteristics) and may take about 2 minutes to complete. The phantom test provides means for users to verify that the Accudxa2® densitometer is maintaining its highest level of performance. Internally, both calibration and quality control may be performed each time the unit is turned on. More frequently, medical practitioners are being asked by insurance companies to provide quality control printouts for their diagnostic devices. Accordingly, when performing a phantom test, users may automatically be prompted to print a QC test report. A QC Phantom Test may be required to be performed after every 300 BMD tests have been completed.

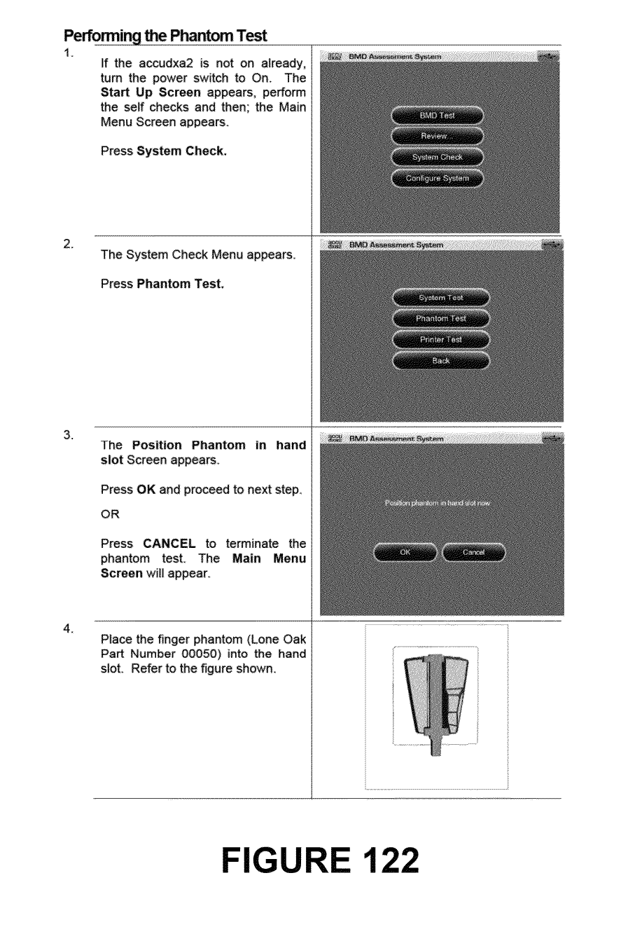

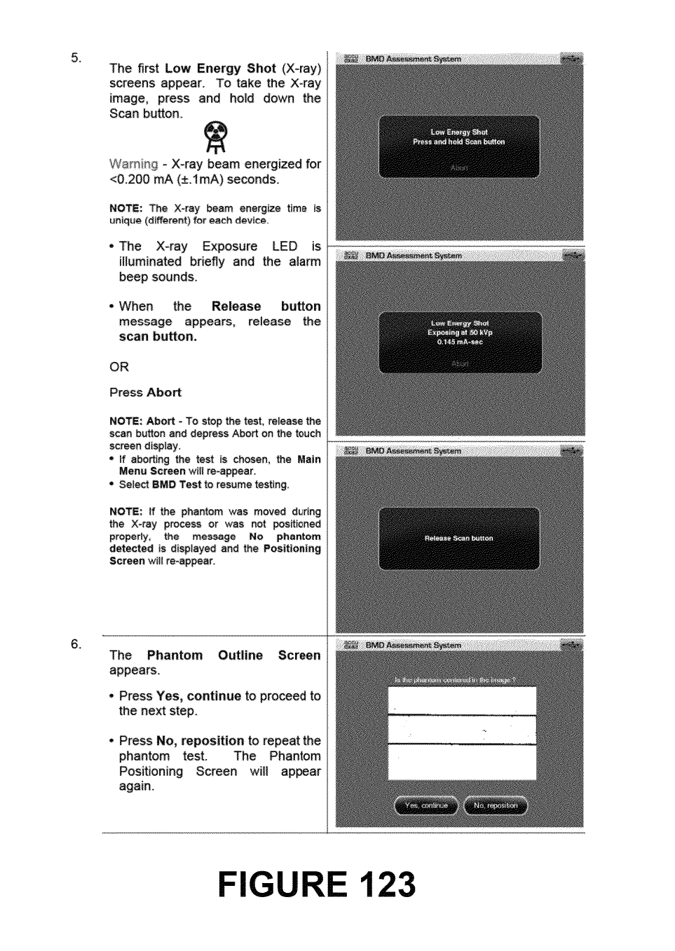

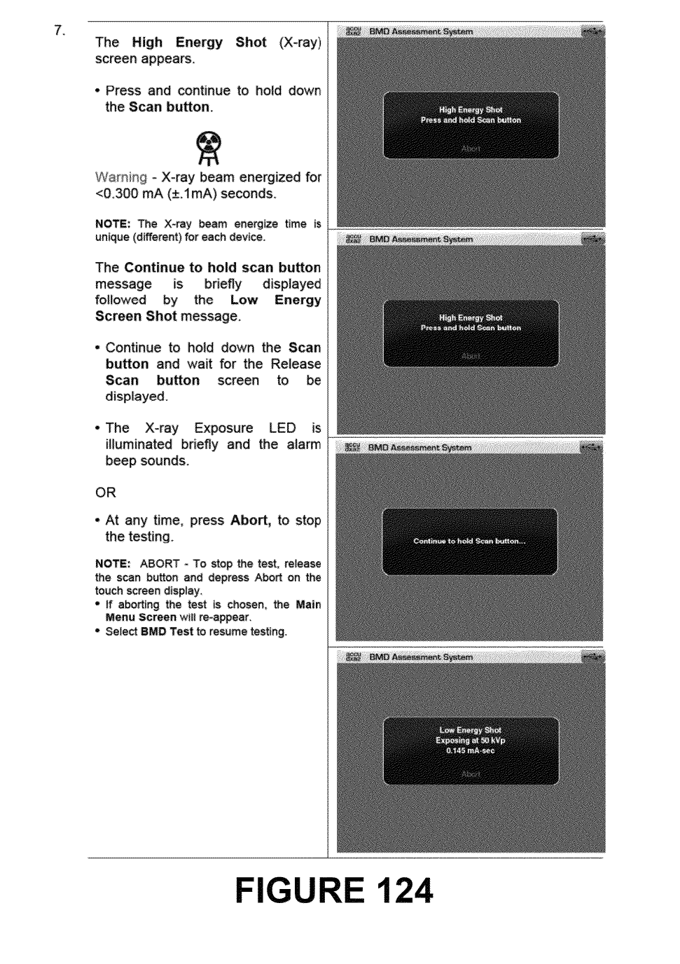

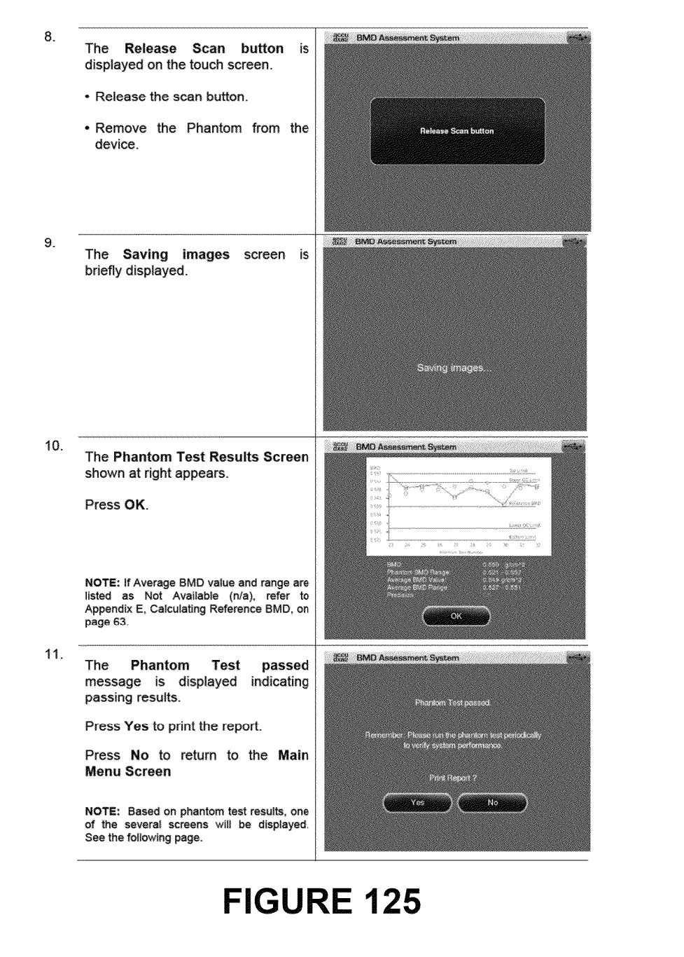

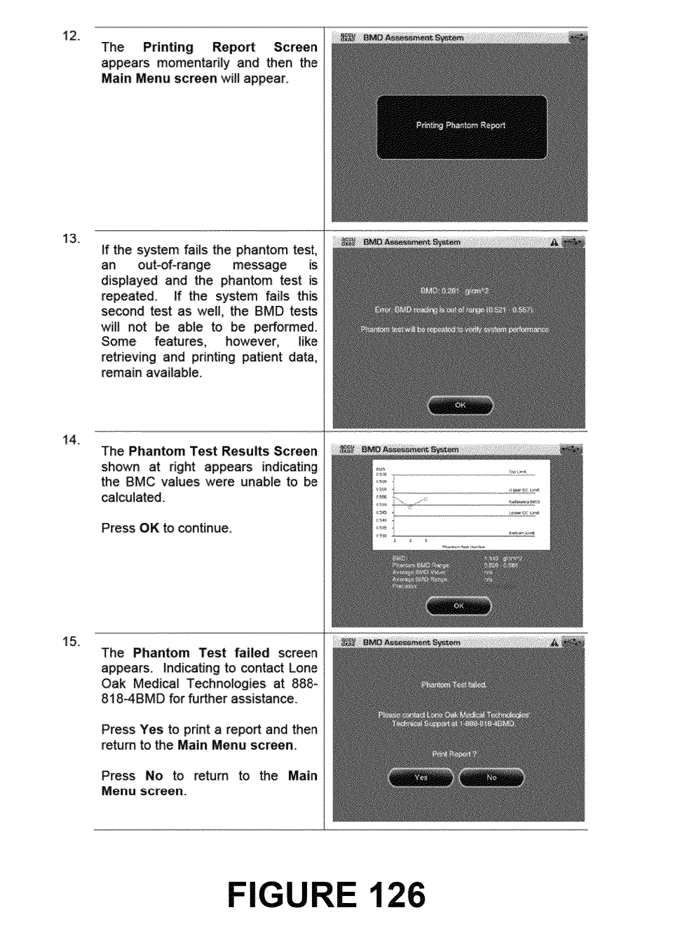

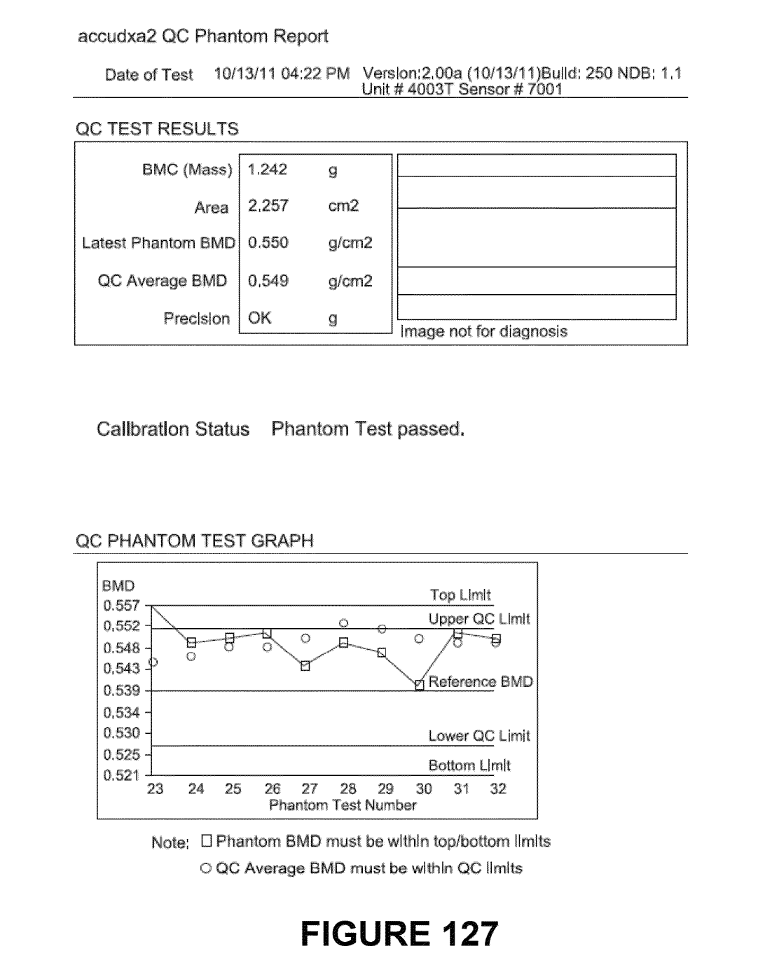

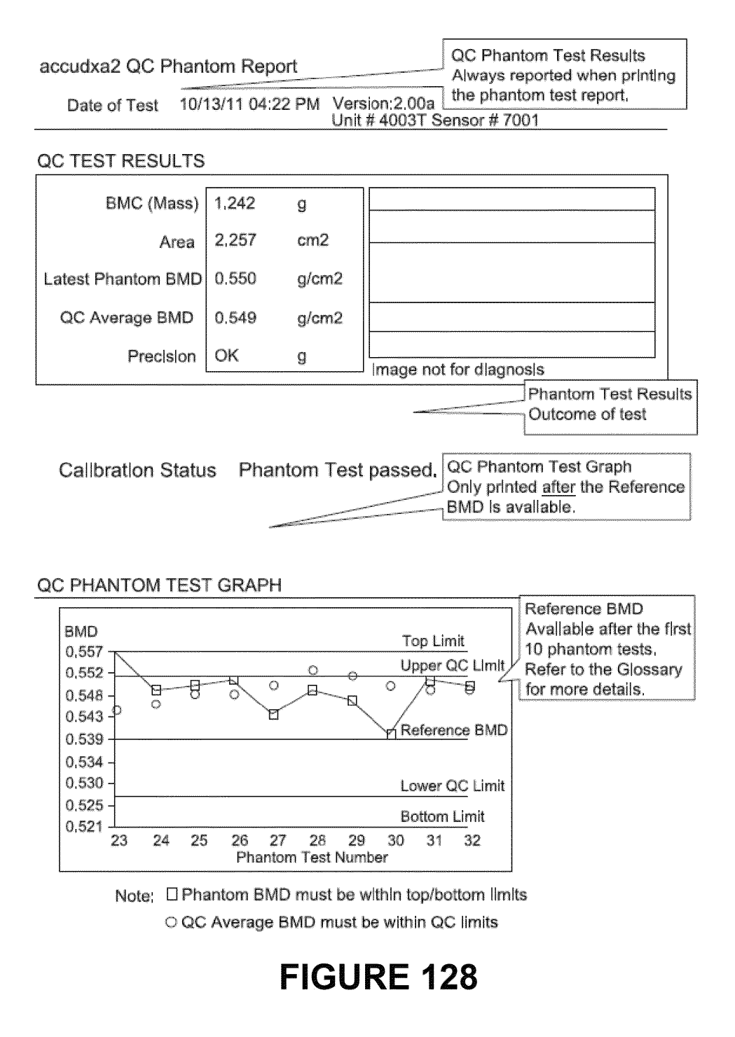

FIG. 122 through FIG. 126 illustrate an example process for performing a phantom test. FIGS. 127 and 128 depict example phantom test reports. A phantom test report may comprise information about system performance. This information may be grouped in two areas: QC Phantom Test Results and QC Phantom Test Graph. Referring to FIG. 127 the QC Phantom Test Results table summarizes the results from the current phantom test and provides other information on the status of BMD testing. The result of the current phantom test is called Phantom BMD and is an indicator of how well the system compares to pre-defined limits in Accudxa2®'s densitometer configuration file. This is one measure of system performance. A second measure of performance may comprise QC Average BMD, which considers both the current and previous Phantom Test results. QC Average BMD is a “moving average”—the result of averaging the last 10 Phantom BMD values. For this reason QC Average BMD may be an indicator of how closely the system is performing to its baseline value (Reference BMD). The QC Phantom Test Graph is plotted below the test results table. The QC Phantom Test Graph may be displayed on the Phantom Test Results Screen on the LCD screen. Printing the phantom report may aid in reviewing the graph. Looking at the QC Phantom Test Graph, certain trends may be observed regarding Phantom BMD and QC Average BMD results. The x-axis in the middle of the table (Reference BMD) provides the guideline for interpreting these results. When the system is performing properly, Phantom BMD values (shown as small squares on the graph) may fall within Phantom limits and QC Average BMD values (shown as small circles) may fall within QC Limits. (Limits are specified in the configuration file.). When both Phantom BMD and QC Average BMD are within the limits for the system, the precision for the unit may be considered satisfactory and is reported as OK. If precision is listed as “Out of Range”, it means that the BMD result may be outside the 0.52 and 0.58 range for acceptable results. In this event, users may be prompted for additional action.

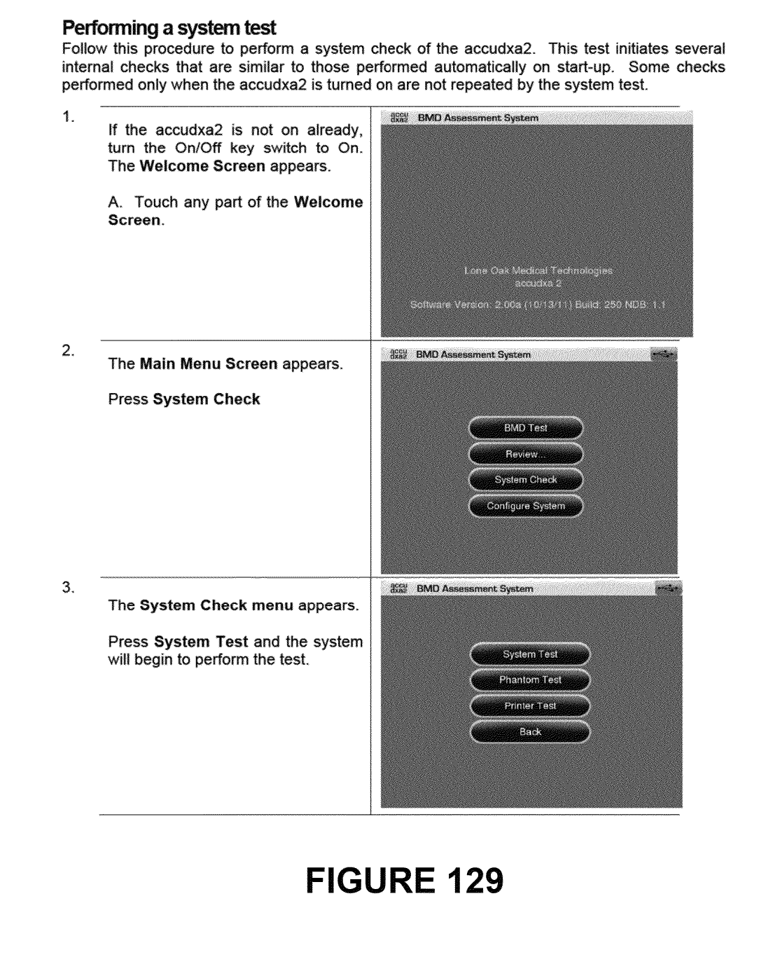

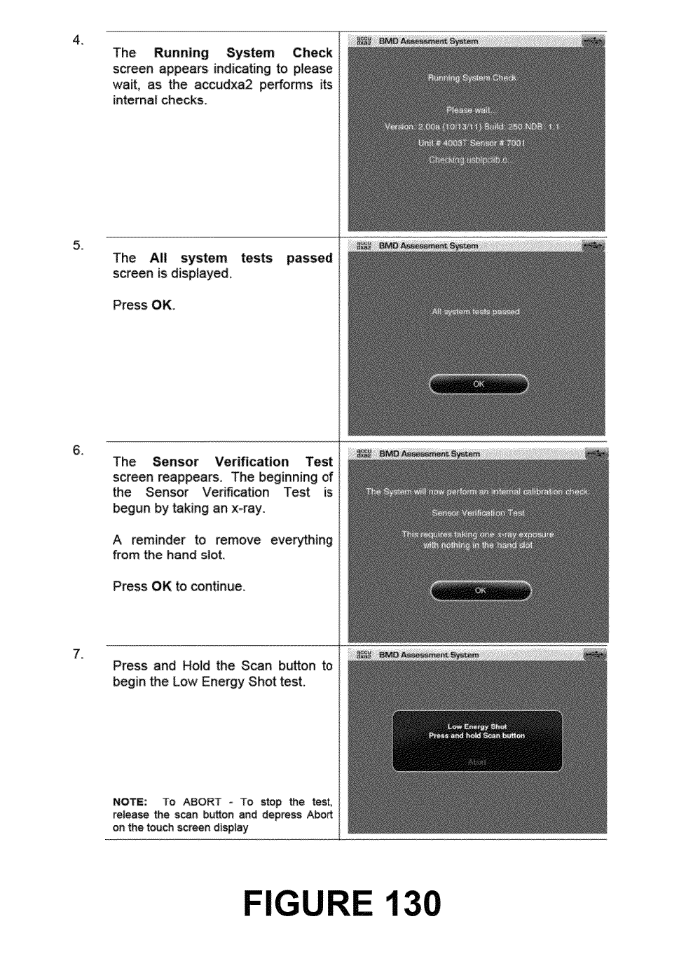

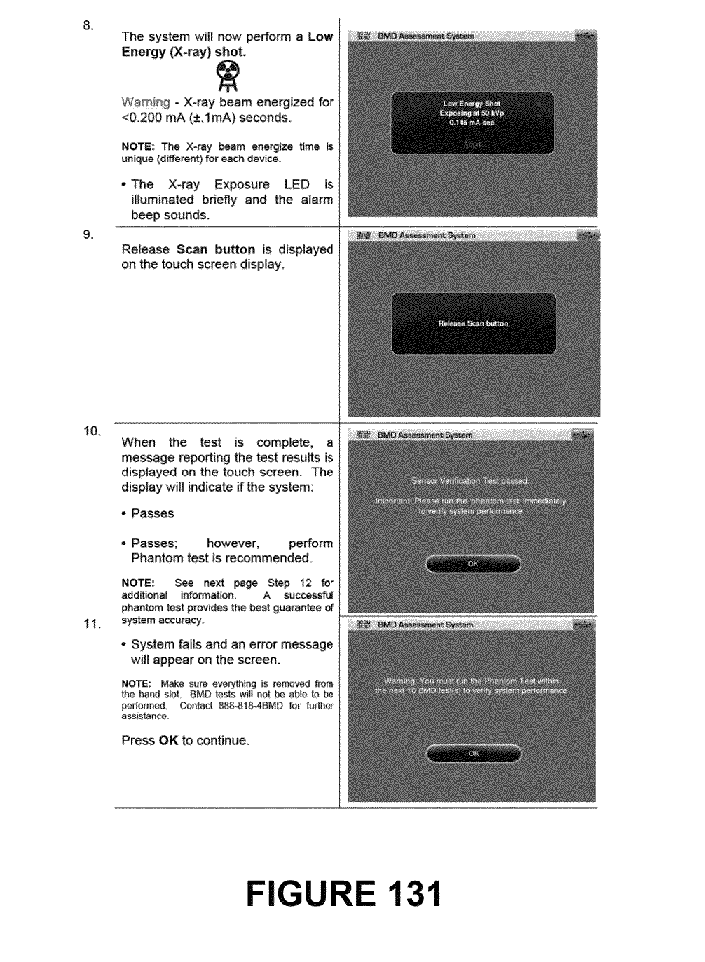

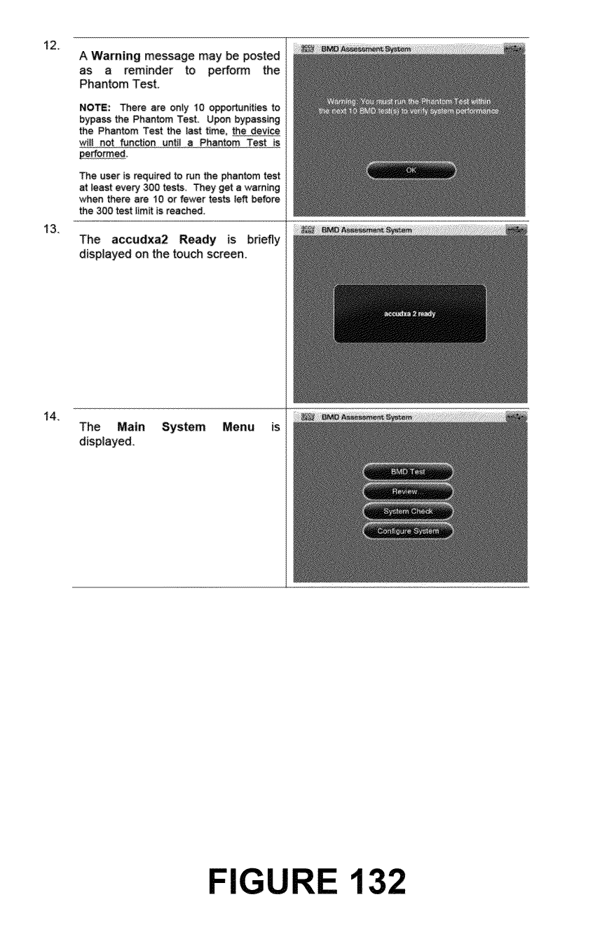

FIG. 129 through 132 depict an example process for performing a system test. A system test may initiate internal checks that may be similar to those performed automatically upon system start-up. Some checks may be performed upon system startup and not repeated during a system test.

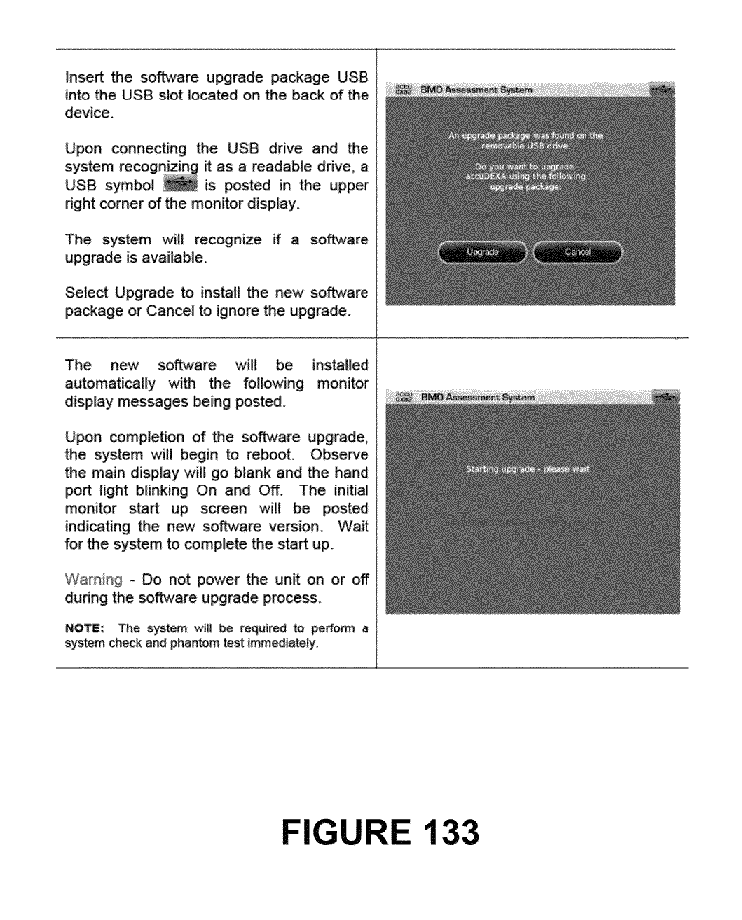

FIG. 133 depicts an example process for performing a software upgrade of the Accudxa2®. A software upgrade package may be downloaded from a web site and written to a USB Thumb Drive formatted as an NTFS or similar file system storage device. A USB Thumb Drive bearing an Accudxa2® software upgrade package may be inserted into one of two slots on the back of the Accudxa2®. The Accudxa2® software may detect that an upgrade package exists on a USB Thumb Drive connected to the Accudxa2® and may use an LCD display to prompt the device operator to Upgrade or Cancel the upgrade of the Accudxa2® software. The device operator may select Upgrade on an LCD touchscreen panel to initiate an upgrade of the accudxa software.

The Accudxa2® densitometer may perform an automatic check of its ability to operate whenever it is turned on. Components verified by this check may include software executable and system files, sensors and interfaces, and mechanical fixtures. If the device fails the system check, an error message may appear on the screen display, listing the cause of the problem. For example, if normal operating temperature limits are exceeded, the system may report, Error: System temperature too hot (70-85 F/21-29 C only) or Error: System temperature too cold (70-85 F/21-29 C only) as appropriate. A system test may be performed at any time. Other user initiated system tests that may be initiated via the system check menu may include System Test, Printer Test, and Phantom Test.

The Accudxa2® densitometer may estimate bone mineral content (BMC, g) and bone mineral density (BMD, g/cm2) in a region of the middle phalanx of the third finger of the non-dominant hand using dual-energy X-ray absorptiometry (DEXA). The density of soft tissue may be compensated for by acquiring information at two distinct energy levels. The Accudxa2® densitometer may emit a low-energy X-ray pulse at 50 kVp and a high-energy X-ray pulse at 70 kVp. At 70 kVp, a zinc plate may be used to filter out the low energy X-rays. A poly methyl methacrylate (PMMA) and an 1100-grade aluminum finger wedge of known density may be aligned in the field of view (FOV) of the sensor. The known density of the wedge may be used in bone density estimation, allowing for a relationship to be established between X-ray attenuation and density, which may be applied to every pixel of the X-ray sensor in the FOV. Furthermore, inclusion of the wedge within the FOV may allow a calibration test to be performed during each exam.

The x-ray mechanism of the densitometer may utilize a duty cycle as depicted in FIG. 76. A densitometer pulse may last approximately 0.15 seconds, which is equivalent to 8.4 pulses as indicated by the x in the FIG. 76. The densitometer may utilize two X-ray exposures as depicted in the table below.

| Impulse | Duration |
|---|---|
| High Energy | .09 seconds (maximum) |
| Low Energy | .06 seconds (maximum) |

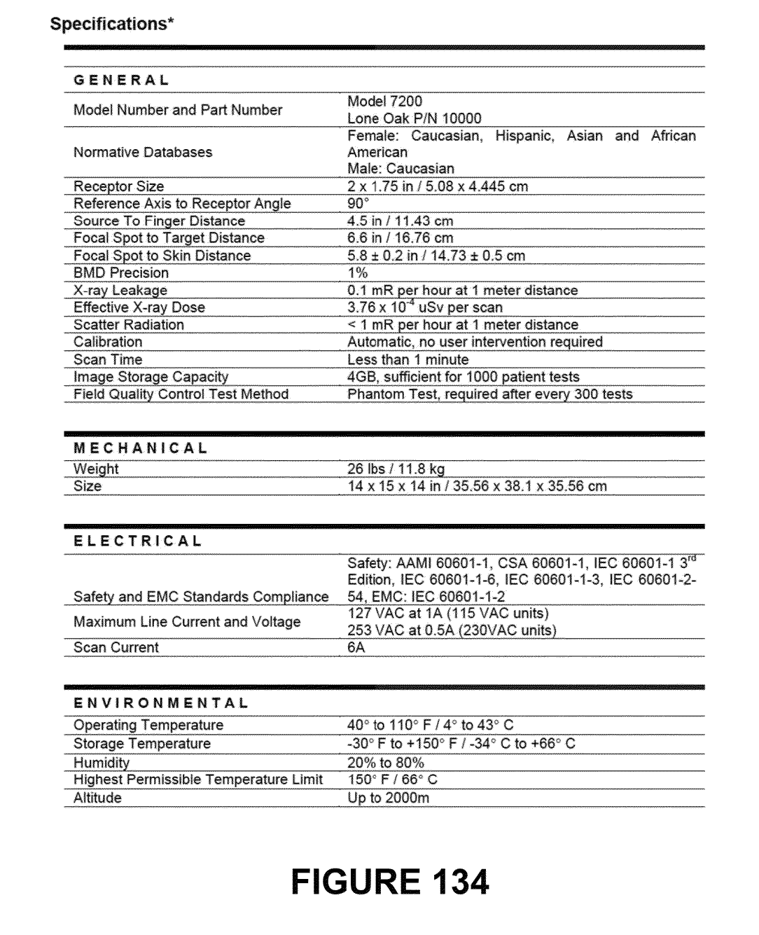

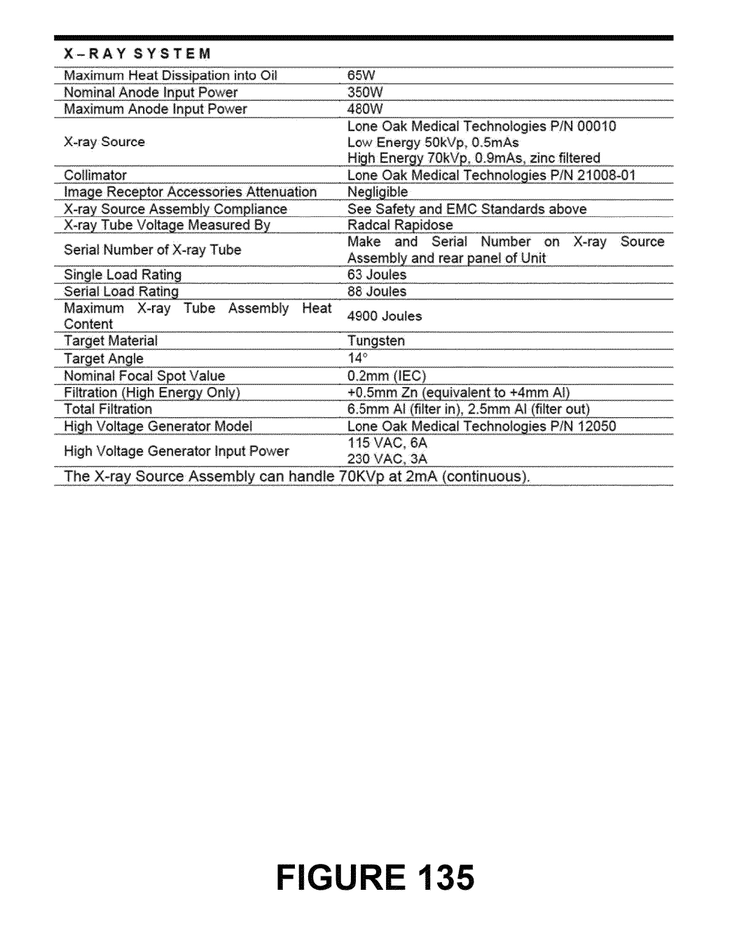

In an example configuration, the densitometer may be embodied in accordance with the example specifications and operate in accordance with the electrical summary depicted in FIG. 134 and FIG. 135.

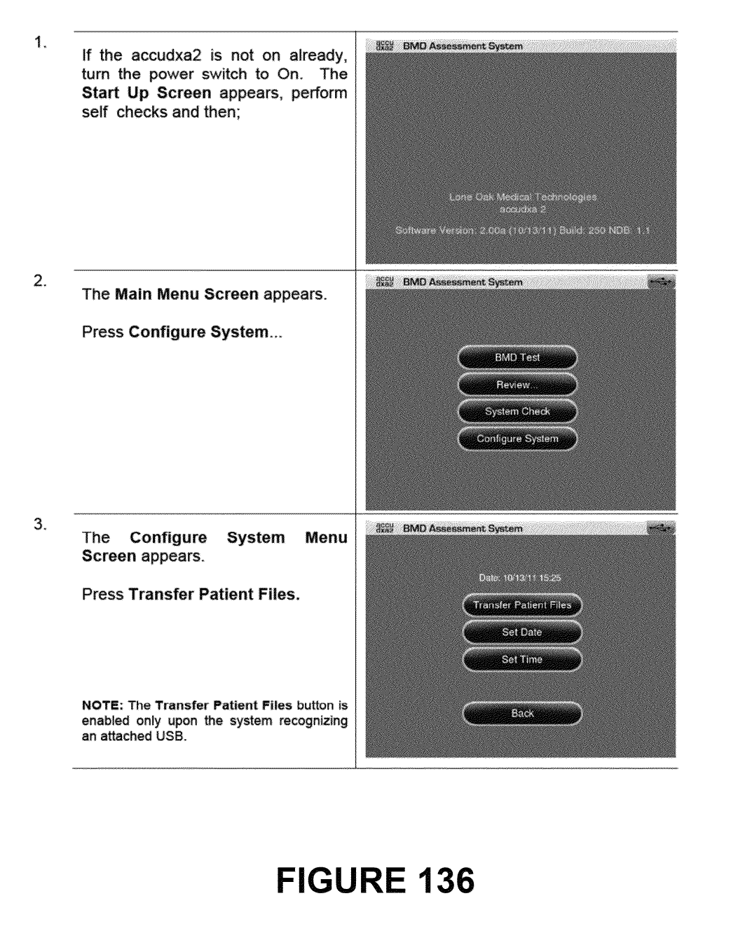

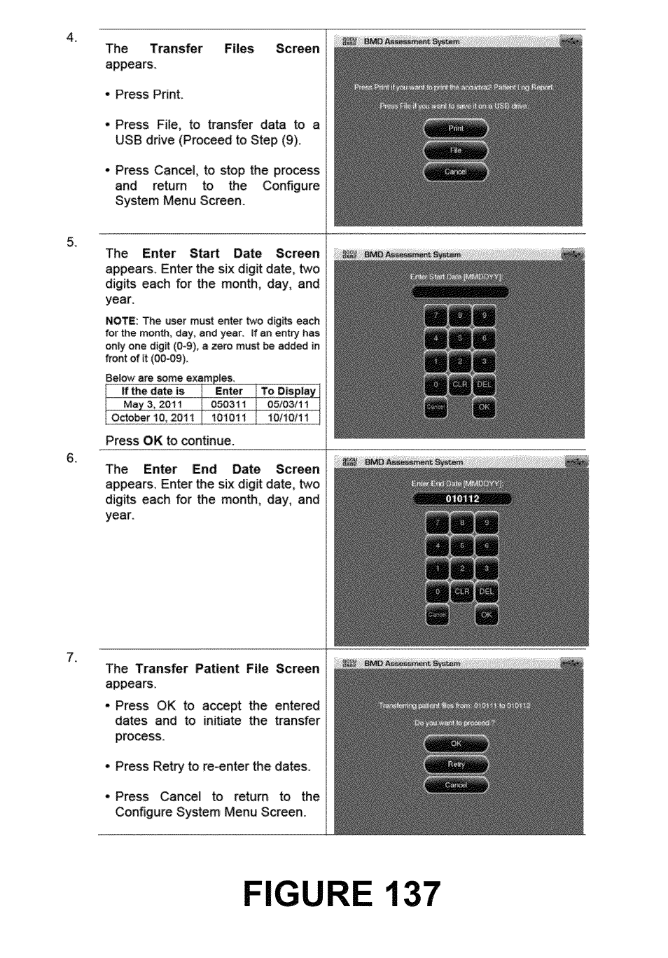

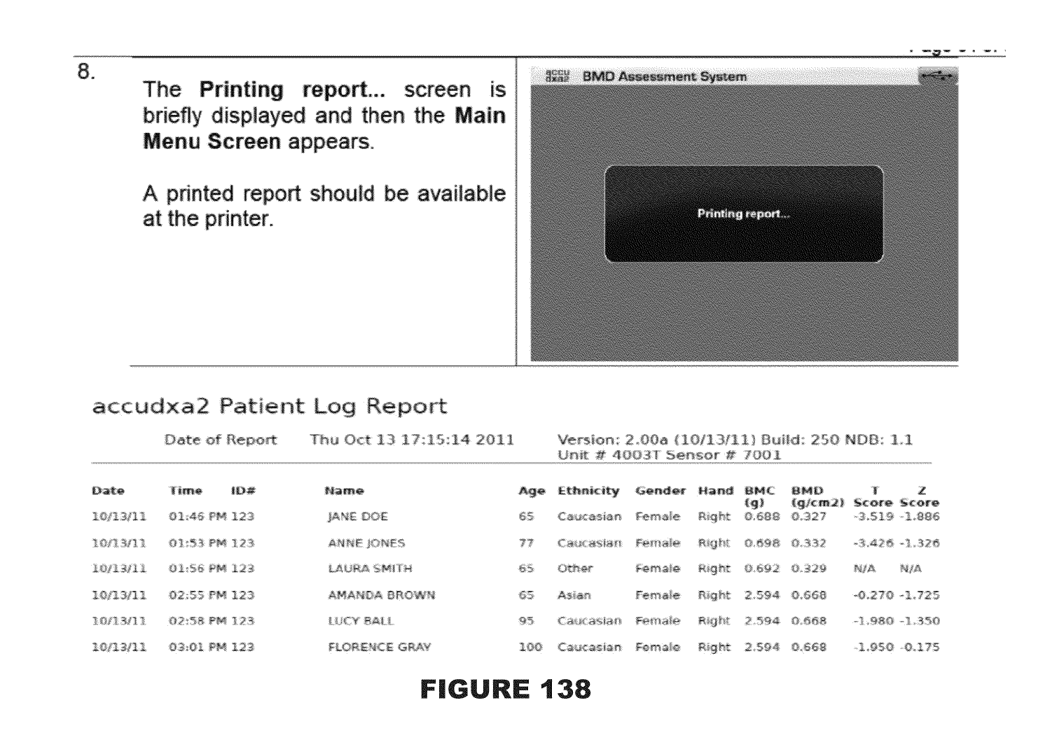

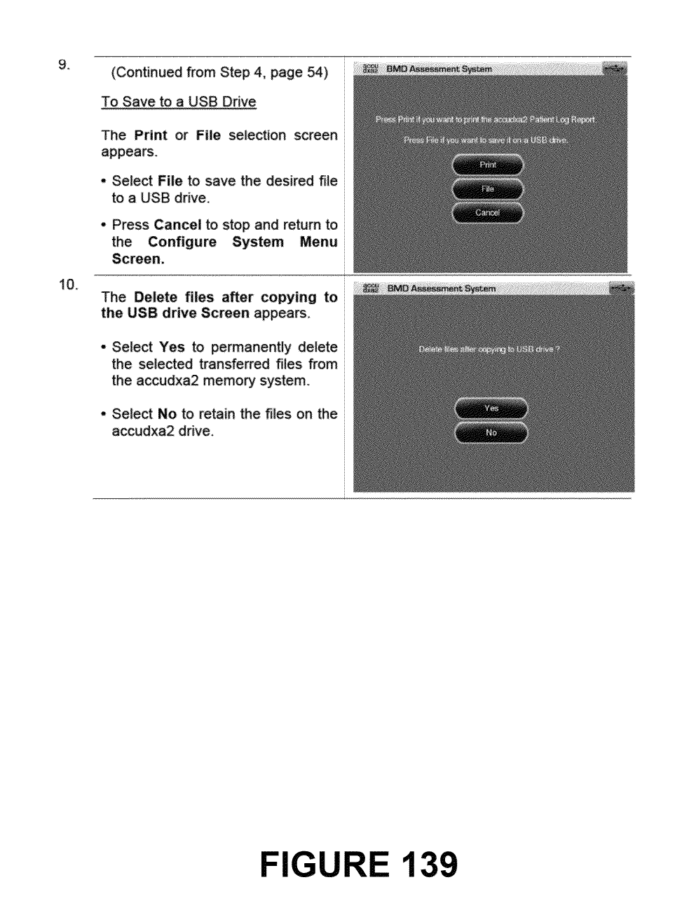

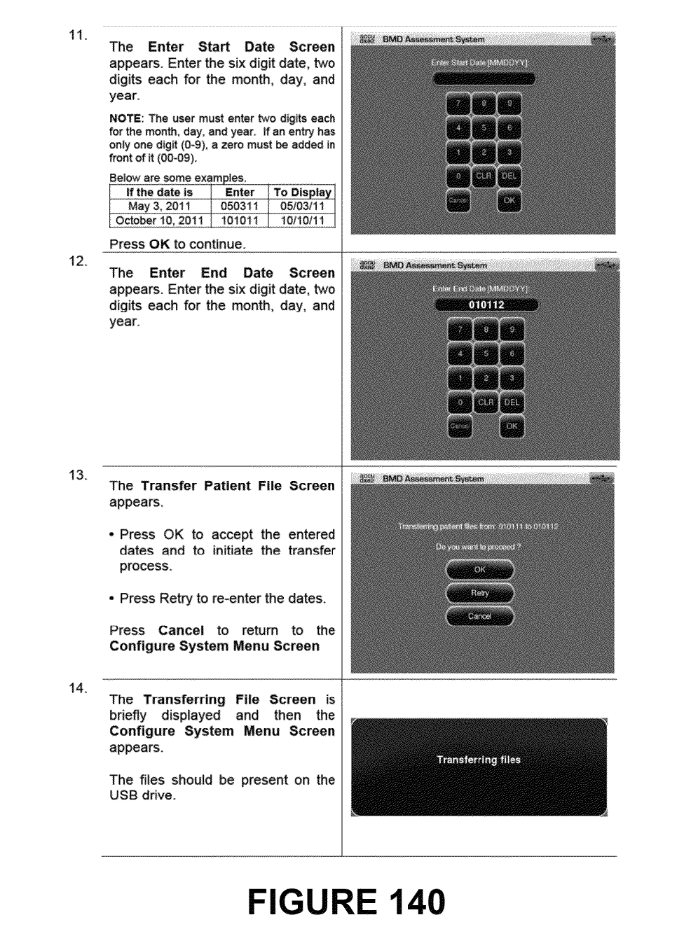

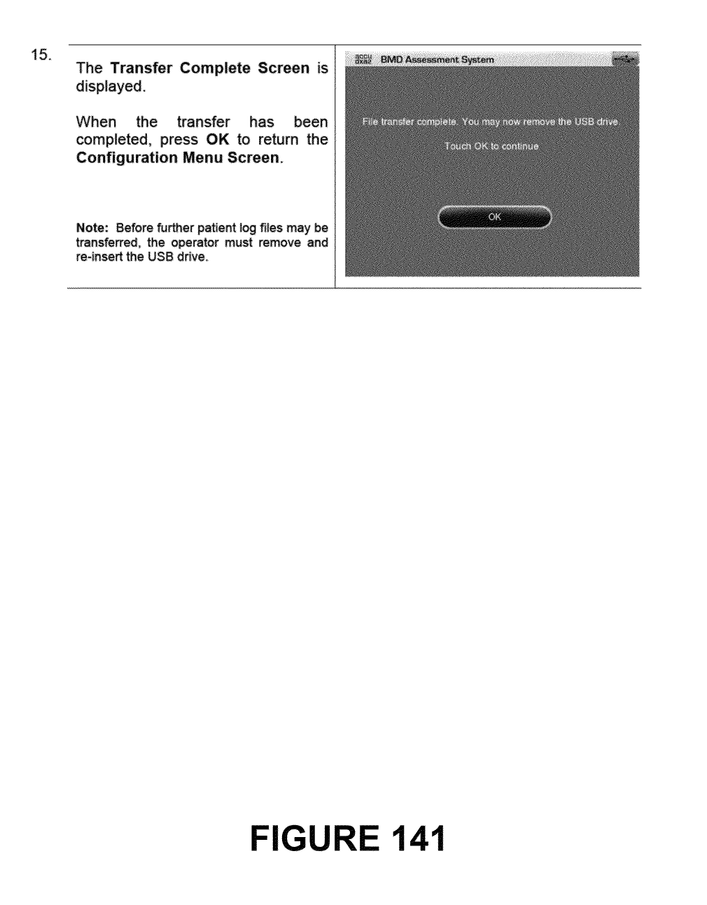

FIG. 136 through FIG. 138 illustrate an example process for printing a patient log report. A patient log report may comprise patient information, BMD and BMC scores, and/or t- and z-scores. (X-ray images and BMD report graphs are not included.). After performing the procedure, a single log file may be generated including test results from the range of dates (one day or many) specified by the user. FIG. 139 through FIG. 141 illustrate an example process for copying a patient log report. The patient log report may be copied into a spreadsheet, a document, file, or the like. The patient log report may be copied into any appropriate format, such as, for example, EXCEL®, WORD®, NOTEPAD®, or the like.

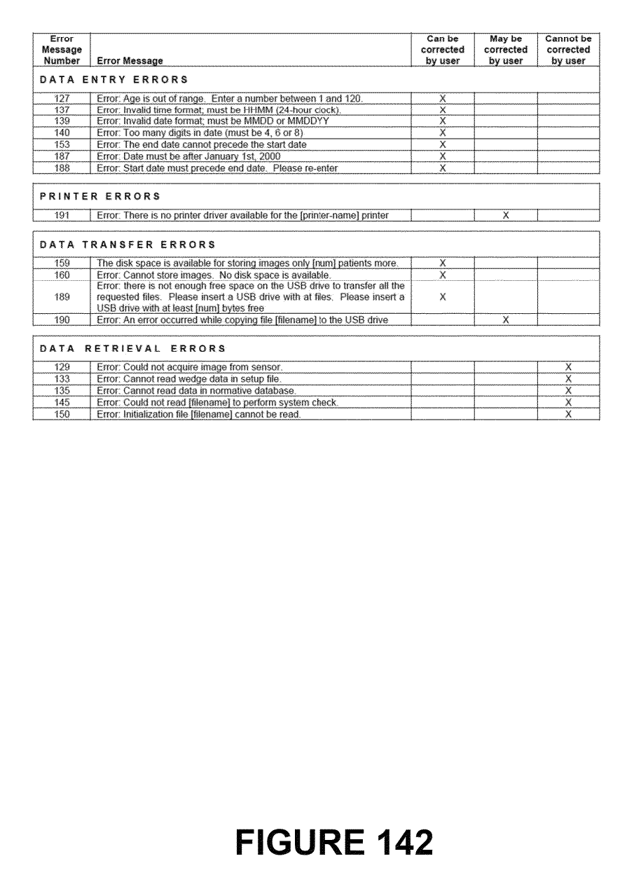

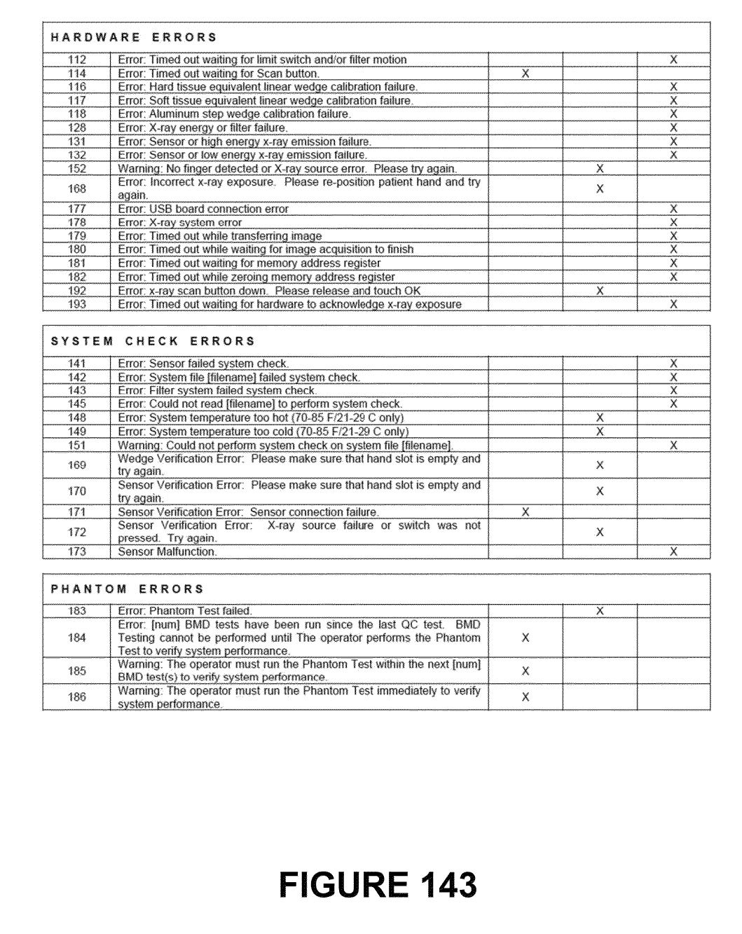

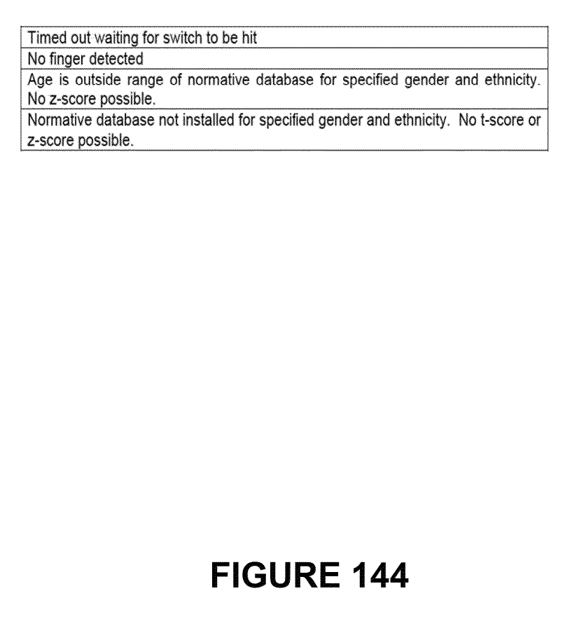

FIG. 142 through FIG. 144 depict example error messages.

While example embodiments of the herein described densitometer have been described in connection with various computing devices, components, and processors, the underlying concepts may be applied to any appropriate computing devices, components, and processors capable of implementing the herein described densitometer. The various techniques described herein may be implemented in connection with any appropriate hardware and software. Thus, the methods and apparatuses for the herein described densitometer, or certain aspects or portions thereof, may implement program code (i.e., instructions) embodied in tangible and/or other media that is not a signal (not a propagating signal, not a transient signal), such as floppy diskettes, CD-ROMs, hard drives, or any other tangible machine-readable storage medium, wherein, when the program code is loaded into and executed by a machine, such as a computer, processor, or the like, the machine becomes an apparatus for implementing the herein described densitometer. In the case of program code execution on programmable computers, the computing device may include a processor, a storage medium readable by the processor (including volatile and non-volatile memory and/or storage elements), at least one input device, and at least one output device. The program(s) can be implemented in assembly or machine language, if desired. The language can be a compiled or interpreted language, and combined with hardware implementations.

Methods and systems for usage notification may also be practiced via communications embodied in the form of program code that may be transmitted over some transmission medium, such as over electrical wiring or cabling, through fiber optics, or via any other form of transmission, wherein, when the program code is received, loaded into, and executed by a machine, such as an EPROM, a gate array, a programmable logic device (PLD), a client computer, or the like, the machine becomes an apparatus for usage notification. When implemented on a general-purpose processor, the program code combines with the processor to provide a unique apparatus that operates to invoke the functionality of usage notification as described herein. Additionally, any storage techniques used in connection with a usage notification system may be a combination of hardware and software.

While usage the herein described densitometer has been described in connection with the various embodiments of the various figures, it is to be understood that other similar embodiments may be used or modifications and additions may be made to the described embodiments for performing the same function of the herein described densitometer without deviating therefrom. Therefore, the herein described densitometer should not be limited to any single embodiment, but rather should be construed in breadth and scope in accordance with the appended claims.

What is claimed is:

1. A apparatus comprising:
a first alignment portion;
a second alignment portion; and
a recessed portion attached to and positioned between the first alignment portion and the second alignment portion, wherein a surface of the recessed portion is shaped to conform to a shape of an object for positioning the object within the recessed portion.

2. The apparatus of claim 1, further comprising a plate for facilitating positioning of the object within the recessed portion.

3. The apparatus of claim 1, wherein the surface of the recessed portion is concave to conform to a shape of the object.

4. The apparatus of claim 1, wherein the object comprises a body part.

5. The apparatus of claim 1, wherein the object comprises a finger.

6. The apparatus of claim 1, wherein:
the object comprises a finger of a hand; and
the plate is configured to support a palm of the hand.

7. The apparatus of claim 1, wherein the object comprises a middle finger.

8. The apparatus of claim 1, wherein the object comprises a finger of a non-dominant hand.

9. The apparatus of claim 1, wherein the apparatus is configured to facilitate alignment of a region of the object within the apparatus.

10. The apparatus of claim 9, wherein:
the object comprises a finger; and
the region comprises a joint between an intermediate phalanx and a proximal phalanx of the finger.

11. The apparatus of claim 10, further comprising a light projector for projecting visible light, wherein:
the visible light is projected at a predetermined portion of the apparatus; and
alignment is accomplished when the joint between the intermediate phalanx and the proximal phalanx of the finger is illuminated by the visible light.

12. The apparatus of claim 11, wherein the light projector comprises a laser.

13. The apparatus of claim 1, further comprising:
an energy source; and
an imaging sensor, wherein utilization of the energy source and the imaging sensor facilitates a determination of bone mineral density of the object.

14. A method comprising:
projecting visible light at a predetermined portion of an apparatus, the apparatus comprising:
a first alignment portion;
a second alignment portion; and
a recessed portion attached to and positioned between the first alignment portion and the second alignment portion, wherein a surface of the recessed portion is shaped to conform to a shape of an object;
adjusting a position of an object placed within the recessed portion of the apparatus until a predetermined portion of the object is illuminated by the visible light.

15. The method of claim 14, wherein the object comprises a middle finger.

16. The method of claim 14, wherein the object comprises a finger of a non-dominant hand.

17. The method of claim 14, wherein:
the object comprises a finger; and
the predetermined portion of the finger comprises a joint between an intermediate phalanx and a proximal phalanx of the finger.

18. The method of claim 14, wherein the visible light projector comprises laser light.

19. The method of claim 14, further comprising determining a bone mineral density of the object.

20. A computer readable storage comprising executable instructions that when executed by a processor cause the processor to effectuate operations comprising:
projecting visible light at a predetermined portion of an apparatus, the apparatus comprising:
a first alignment portion;
a second alignment portion; and
a recessed portion attached to and positioned between the first alignment portion and the second alignment portion, wherein a surface of the recessed portion is shaped to conform to a shape of an object;
placing the object within the recessed portion of the apparatus;
adjusting a position of an object placed within the recessed portion of the apparatus until a predetermined portion of the object is illuminated by the visible light.
